(19) United States
(12) Patent Application Publication
(10) Pub. No.: US 2022/0402908 A1
(43) Pub. Date: Dec. 22, 2022

(54) CXCR6 INHIBITORS AND METHODS OF USE

(71) Applicant: Sanford Burnham Prebys Medical Discovery Institute, La Jolla, CA (US)

(72) Inventors: Satyamaheshwar PEDDIBHOTLA, Orlando, FL (US); Paul M. HERSHBERGER, Satellite Beach, FL (US); Richard Jason KIRBY, Newton, MA (US); Siobhan MALANY, Orlando, FL (US); Layton H. SMITH, Orlando, FL (US); Patrick R. MALONEY, Orlando, FL (US); Hampton SESSIONS, Orlando, FL (US); Daniela DIVLIANSKA, Orlando, FL (US); Anthony B. PINKERTON, Rancho Santa Fe, CA (US)

(21) Appl. No.: 17/625,192

(22) PCT Filed: Jul. 7, 2020
(86) PCT No.: PCT/US2020/041005

§ 371 Date: Jan. 6, 2022

Related U.S. Application Data
(60) Provisional application No. 62/871,616, filed on Jul. 8, 2019.

(51) Int. Cl. C07D 451/14

(52) U.S. Cl. CPC C07D 451/14

(57) ABSTRACT

Provided herein are small molecule inhibitors of CXCR6 receptor, compositions comprising the compounds, and methods of using the compounds and compositions. The compounds are 9-azbicyclo[3.3.1]nonane or 9-diazbicyclo[3.3.1]nonane derivatives, whose synthesis is also described. Also provided are method of treating a disease or condition (such as, cancer) mediated by CXCR6/CXCL16 signaling pathway in a mammal.

### CROSS-REFERENCE

This application claims benefit of U.S. Provisional Application No. 62/871,616, filed on Jul. 8, 2019, which is herein incorporated by reference in its entirety.

### STATEMENT AS TO FEDERALLY SPONSORED RESEARCH

The invention was made with government support under R03 MH095589 awarded by the National Institutes of Health and under Grant No. 2016.6-HTL-0006 awarded by the Florida Department of Health. The government has certain rights in the invention.

### BACKGROUND OF THE DISCLOSURE

Many medically significant biological processes are mediated by proteins participating in signal transduction pathways that involve G-proteins and/or second messengers such as cAMP. The membrane protein gene superfamily of G-protein coupled receptors (GPCRs) includes a wide range of biologically active receptors, such as hormone, viral, growth factor and neuroreceptors. Chemokines are chemotactic cytokines that are released by a wide variety of cells to attract macrophages, T cells, eosinophils, basophils and neutrophils. Chemokine (C—X—C) receptor 6 (CXCR6) is a member of the chemokines receptor subfamily of GPCRs.

### SUMMARY OF THE DISCLOSURE

Described herein are compounds that are CXCR6 receptor inhibitors. Also described herein and compositions and methods of using the CXCR6 receptor inhibitors.

In one aspect, described herein is a compound that has the structure of Formula (I), or a pharmaceutically acceptable salt, solvate, or N-oxide thereof:

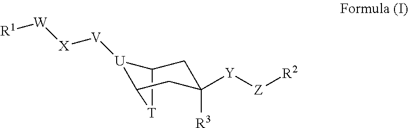

wherein,
U is N or CR⁴;
W, X, V, T, Y, and Z are independently absent, —O—, —S—, —S(═O)—, —S(═O)₂—, —(CR⁴R⁴)ₙ—, —C(═O)—, —C(═NR⁴)—, —C(═O)O—, —OC(═O)—, —C(═O)C(═O)—, —C(═O)NR⁴—, —NR⁴C(═O)—, —OC(═O)NR⁴—, —NR⁴C(═O)O—, —NR⁴C(═O)NR⁴—, —S(═O)₂NR⁴—, —NR⁴S(═O)₂—, —NR⁴—;
R¹ and R² are independently selected from the group consisting of H, D, halogen, —CN, —OR⁴, —SR⁴, —S(═O)R⁴, —S(═O)₂R⁴, —N(R⁴)₂, —NR⁴S(═O)(═NR⁴)R⁴, —NR⁴S(═O)₂R⁴, —S(═O)₂N(R⁴)₂, —C(═O)R⁴, —OC(═O)R⁴, —C(═O)OR⁵, —OC(═O)OR⁵, —C(═O)N(R⁴)₂, —OC(═O)N(R⁴)₂, —NR⁴C(═O)R⁴, substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, and substituted or unsubstituted heteroaryl;
or R¹ is an unsubstituted bicyclic heteroaryl or substituted bicyclic heteroaryl that is substituted with 1, 2, 3, 4, or 5 R⁶ and optionally 1 R^{6A};
or R² is an unsubstituted phenyl or a substituted phenyl that is substituted with 1, 2, 3, 4, or 5 R⁷;
R³ is H, D, F, substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, or substituted or unsubstituted heteroaryl;
   optionally R² and R³ are taken together with the intervening atoms to form a substituted or unsubstituted C₅-C₇ cycloalkyl or a substituted or unsubstituted C₃-C₆ heterocycloalkyl;
each R⁴ is independently H, D, substituted or unsubstituted C₁-C₆ alkyl, substituted or unsubstituted C₁-C₆ fluoroalkyl, substituted or unsubstituted aryl, substituted or unsubstituted monocyclic heteroaryl, —OR⁵, —N(R⁵)₂, —CH₂OR⁵, —CN, —C(═O)OR⁵, —OC(═O)R⁵, —C(═O)N(R⁵)₂, or —NR⁵C(═O)R⁵;
each R⁵ is independently H, D, F, substituted or unsubstituted C₁-C₆ alkyl, substituted or unsubstituted C₁-C₆ fluoroalkyl, substituted or unsubstituted C₁-C₆ heteroalkyl, substituted or unsubstituted aryl, or substituted or unsubstituted heteroaryl;
n is 1, 2, 3, or 4;
each R⁶ is independently D, halogen, substituted or unsubstituted C₁-C₄ alkyl, substituted or unsubstituted C₁-C₄ fluoroalkyl, or substituted or unsubstituted C₁-C₄ heteroalkyl;
R^{6A} is H, D, substituted or unsubstituted C₁-C₄ alkyl, substituted or unsubstituted C₁-C₄ fluoroalkyl, or substituted or unsubstituted C₁-C₄ heteroalkyl; and
each R⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, or substituted or unsubstituted C₂-C₉ heterocycloalkyl;
with the provision that when —W—X—V— is —NHC(═O)CH₂—, then R¹ is not 2-chlorophenyl.

In another aspect, described herein is a compound of Formula (II), or a pharmaceutically acceptable salt, solvate, prodrug, or N-oxide thereof:

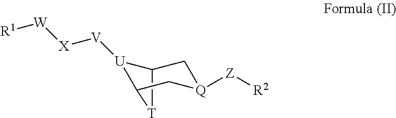

wherein,
U and Q are independently N or CR⁴;
W, X, V, T, and Z are independently absent, —O—, —S—, —S(═O)—, —S(═O)₂—, —(CR⁴R⁴)ₙ—, —C(═O)—, —C(═NR⁴)—, —C(═O)O—, —OC(═O)—, —C(═O)C(═O)—, —C(═O)NR⁴—, —NR⁴C(═O)—, —OC(═O)NR⁴—, —NR⁴C(═O)O—, —NR⁴C(═O)NR⁴—, —S(═O)₂NR⁴—, —NR⁴S(═O)₂—, —NR⁴—;
R¹ and R² are independently selected from the group consisting of H, D, halogen, —CN, —OR⁴, —SR⁴, —S(═O)R⁴, —S(═O)₂R⁴, —N(R⁴)₂, —NR⁴S(═O)(═NR⁴)R⁴, —NR⁴S(═O)₂R⁴, —S(═O)₂N(R⁴)₂, —C(═O)R⁴, —OC(═O)R⁴, —C(═O)OR⁵, —OC(═O)OR⁵, —C(═O)N(R⁴)₂, —OC(═O)N(R⁴)₂, —NR⁴C(═O)R⁴, substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, and substituted or unsubstituted heteroaryl;
or R¹ is an unsubstituted bicyclic heteroaryl or substituted bicyclic heteroaryl that is substituted with 1, 2, 3, 4, or 5 R⁶ and optionally 1 R^{6A};
or R² is an unsubstituted phenyl or a substituted phenyl that is substituted with 1, 2, 3, 4, or 5 R⁷;
each R⁴ is independently H, D, substituted or unsubstituted C₁-C₆ alkyl, substituted or unsubstituted C₁-C₆ fluoroalkyl, substituted or unsubstituted aryl, substituted or unsubstituted monocyclic heteroaryl, —OR⁵, —N(R⁵)₂, —CH₂OR⁵, —CN, —C(═O)OR⁵, —OC(═O)R⁵, —C(═O)N(R⁵)₂, or —NR⁵C(═O)R⁵;
each R⁵ is independently H, D, F, substituted or unsubstituted C₁-C₆ alkyl, substituted or unsubstituted C₁-C₆ fluoroalkyl, substituted or unsubstituted C₁-C₆ heteroalkyl, substituted or unsubstituted aryl, or substituted or unsubstituted heteroaryl; and
n is 1, 2, 3, or 4;
each R⁶ is independently D, halogen, substituted or unsubstituted C₁-C₄ alkyl, substituted or unsubstituted C₁-C₄ fluoroalkyl, or substituted or unsubstituted C₁-C₄ heteroalkyl;
R^{6A} is H, D, substituted or unsubstituted C₁-C₄ alkyl, substituted or unsubstituted C₁-C₄ fluoroalkyl, or substituted or unsubstituted C₁-C₄ heteroalkyl; and
each R⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, or substituted or unsubstituted C₂-C₉ heterocycloalkyl;
with the provision that when —W—X—V— is —NHC(═O)CH₂—, then R¹ is not 2-chlorophenyl.

Any combination of the groups described above or below for the various variables is contemplated herein. Throughout the specification, groups and substituents thereof are chosen by one skilled in the field to provide stable moieties and compounds.

In one aspect, provided herein is a pharmaceutical composition comprising a compound of Formula (I), (Ia), (Ib), (Ic), (Id), (II), or (IIa), or a pharmaceutically acceptable salt thereof, and at least one pharmaceutically acceptable excipient.

In some embodiments, the compound of Formula (I), (Ia), (Ib), (Ic), (Id), (II), or (IIa), or a pharmaceutically acceptable salt thereof, is formulated for administration to a mammal by intravenous administration, subcutaneous administration, oral administration, inhalation, nasal administration, transdermal administration, or ophthalmic administration. In some embodiments, the compound of Formula (I), (Ia), (Ib), (Ic), (Id), (II), or (IIa), or a pharmaceutically acceptable salt thereof, is in the form of a tablet, a pill, a capsule, a liquid, a suspension, a gel, a dispersion, a solution, an emulsion, an ointment, or a lotion.

In one aspect, described herein is a method of treating a disease or condition mediated by CXCR6/CXCL16 signaling pathway in a mammal in need thereof comprising administering a CXCR6 inhibitor compound as described herein, or a pharmaceutically acceptable salt, solvate, or N-oxide thereof, to the mammal in need thereof.

In some embodiments, the disease or condition is cancer.

In some embodiments, the cancer is hepatocellular carcinoma. In some embodiments, the cancer is prostate cancer. In some embodiments, the cancer is gastric adenocarcinoma. In some embodiments, the cancer is bladder cancer. In some embodiments, the cancer is papillary thyroid carcinoma. In some embodiments, the cancer is non-small cell lung carcinoma.

In some embodiments, the disease or condition is autoimmune hepatitis.

In some embodiments, the disease or condition is a kidney injury or lung injury. In some embodiments, the kidney injury is acute kidney injury.

In some embodiments, the disease or condition is a myocardial ischemia or reperfusion injury.

In some embodiments, the disease or condition is an inflammatory disease or condition.

In another aspect, described herein is the use of a CXCR6 inhibitor compound as described herein, or a pharmaceutically acceptable salt, solvate, prodrug, or N-oxide thereof, in the manufacture of a medicament for the treatment or prevention of a disease or condition that is mediated by CXCR6/CXCL16 signaling pathway.

Yet, in another aspect, described herein is use of a CXCR6 inhibitor compound as described herein, or a pharmaceutically acceptable salt, solvate, or N-oxide thereof, in the manufacture of a medicament for the treatment or amelioration of the symptoms of a disease or condition that is mediated by CXCR6/CXCL16 signaling pathway.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates tumor growth reduction for SK-Hep-1 cancer xenograft treated with Compound 1 or cyclophosphamide compare to control.

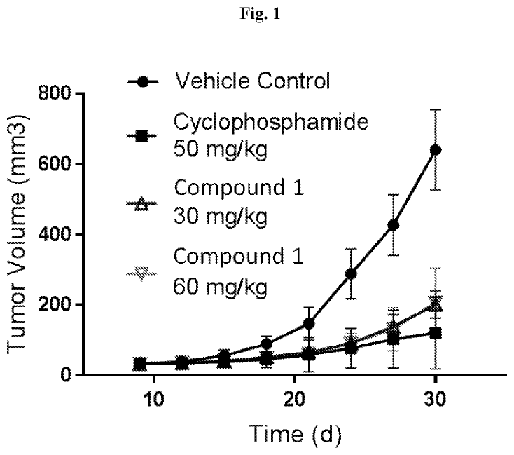

FIG. 2 illustrates tumor size for SK-Hep-1 cancer xenograft treated with Compound 1 or cyclophosphamide compare to control.

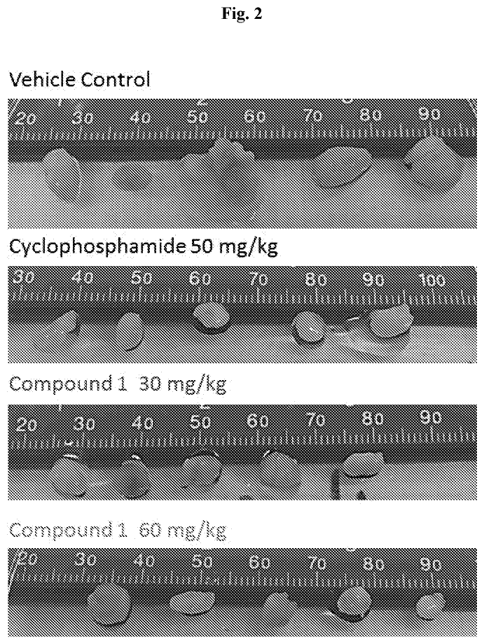

FIG. 3 illustrates tumor weight for SK-Hep-1 cancer xenograft treated with Compound 1 or cyclophosphamide compare to control.

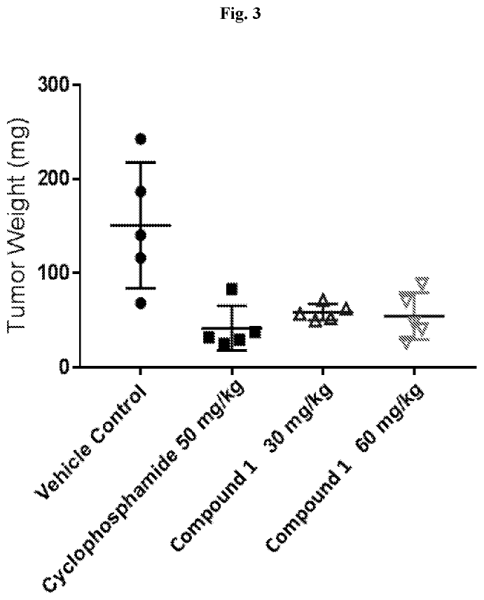

### DETAILED DESCRIPTION OF THE DISCLOSURE

CXCR6 is the seven transmembrane domain G protein-coupled receptor (GPCR) target for the natural ligand, CXCL16, a chemokine that exists in both membrane-anchored and soluble forms. The CXCR6 receptor is a transmembrane protein abundantly expressed on the surface of dendritic cells and by CD4+ cells, CD8+ cells, natural killer (NK) cells and natural killer T (NKT) cells. The CXCR6/CXCL16 axis plays a critical role in pro-inflammatory and pro-fibrotic events in liver and kidney. Knockout mouse studies indicate that the chemokine receptor, CXCR6 and its ligand, CXCL16 contribute to pro-inflammatory cytokine expression in liver and kidney. CXCR6-deficient mice were protected from liver fibrosis and CXCL16 deficiency resulted in protection from hypertensive renal injury and fibrosis.

In chronic liver injury, the production of the soluble form of CXCL16 from sinusoidal epithelial cells is increased. The secretion of CXCL16 promotes NKT cells expressing CXCR6 to migrate to the liver. The transmembrane form of CXCL16 functions as an adhesion molecule, anchoring activated NKT cells3. NKT cells secrete pro-inflammatory cytokines TNF-α and IFN-γ leading to increased levels of CXCL16 and attract more NKT cells in a positive feedback loop. In this way, CXCR6 and its ligand CXCL16 promote liver fibrosis. In mice with diet-induced hepatic injury, the administration of an anti-CXCL16a antibody blocked the accumulation of hepatic NKT cells and pro-inflammatory cytokines.

In liver tissue taken from patients with liver disease, hepatic CXCR6 and CXCL16 mRNA expression is upregulated independent of the underlying etiology of liver disease, such as viral hepatitis, alcoholism, or cholestatic disorders.

CXCL16 expression has been demonstrated in a variety of tissues and cells including activated endothelial cells. Additionally, it was shown that CXCL16 functions as a potent and direct activator of NF-κB and induces KB-dependent pro-inflammatory gene transcription through interaction with heterotrimeric G-proteins triggering downstream PI3K, PDK-1, Akt, and IκB kinase (IKK) signal transduction events. Through a cytokine antibody array, it was shown that CXCL16 protein production was increased in aggressive prostate cancer cells compared to the less aggressive prostate cancer cells or benign prostate cells. It was also found that both IL-1β and TNFα significantly induced CXCL16 production by LNCaP and PC3 cells, thereby indicating inflammatory cytokines may play a role in CXCL16 induction. CXCR6 and CXCL16 are highly expressed in many types of human cancers, including prostate cancer, papillary thyroid carcinoma, non-small cell lung carcinoma, gastric cancer and hepatocellular carcinoma (HCC) and are consistently expressed in hepatoma cell lines. CXCR6 expression profile is low in normal hepatocytes, increases in noninvasive HCC cells, and reaches highest levels in invasive HCCs. Upregulation of CXCR6 receptor contributes to a pro-inflammatory tumor microenvironment that promotes metastasis and has been identified as an independent predictor for increased recurrence and poor survival in patients with HCCs. Knockdown of CXCR6 receptor inhibits HCC cell invasion in vitro and inhibits tumorigenicity, neutrophil recruitment, angiogenesis, and metastasis of hepatoma cells in vivo.

Based on importance of CXCR6 receptor in pro-inflammatory and pro-fibrotic events in liver, kidney, and heart, the compounds which inhibit CXCR6 receptor activity are considered to be useful in treating, amelioration of the symptoms, or preventing inflammation, liver, renal, and heart injury and fibrosis including non-alcoholic fatty liver disease (NAFLD), acute kidney injury, and reperfusion injury. Additionally, small molecule antagonists to CXCR6/CXCL16 signaling may provide a venue to ameliorate tumor progression and metastasis.

In some embodiments, described herein is a CXCR6 receptor inhibitor compound. In some embodiments, described herein is a method of treating a disease or condition mediated by CXCR6/CXCL16 signaling pathway in a mammal in need thereof comprising administering a CXCR6 inhibitor compound as described herein, or a pharmaceutically acceptable salt, solvate, or N-oxide thereof, to the mammal in need thereof.

### Compounds

In one aspect, described herein is a CXCR6 receptor inhibitor that has the structure of Formula (I), or a pharmaceutically acceptable salt, solvate, or N-oxide thereof:

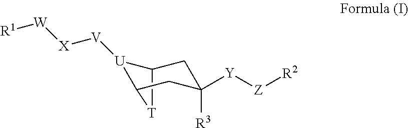

wherein,
U is N or CR⁴;
W, X, V, T, Y, and Z are independently absent, —O—, —S—, —S(═O)—, —S(═O)₂—, —(CR⁴R⁴)ₙ—, —C(═O)—, —C(═NR⁴)—, —C(═O)O—, —OC(═O)—, —C(═O)C(═O)—, —C(═O)NR⁴—, —NR⁴C(═O)—, —C(═S)NR⁴—, —NR⁴C(═S)—, —OC(═O)NR⁴—, —NR⁴C(═O)O—, —OC(═S)NR⁴—, —NR⁴C(═S)O—, —NR⁴C(═O)NR⁴—, NR⁴C(═S)NR⁴—, —S(═O)₂NR⁴—, —NR⁴S(═O)₂—, —NR⁴—;
R¹ and R² are independently selected from the group consisting of H, D, halogen, —CN, —OR⁴, —SR⁴, —S(═O)R⁴, —S(═O)₂R⁴, —N(R⁴)₂, —NR⁴S(═O)(═NR⁴)R⁴, —NR⁴S(═O)₂R⁴, —S(═O)₂N(R⁴)₂, —C(═O)R⁴, —OC(═O)R⁴, —C(═O)OR⁵, —OC(═O)OR⁵, —C(═O)N(R⁴)₂, —OC(═O)N(R⁴)₂, —NR⁴C(═O)R⁴, substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, and substituted or unsubstituted heteroaryl;
R³ is H, D, F, substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, or substituted or unsubstituted heteroaryl; optionally R² and R³ are taken together with the intervening atoms to form a substituted or unsubstituted C₅-C₇ cycloalkyl or a substituted or unsubstituted C₃-C₆ heterocycloalkyl;
each R⁴ is independently H, D, substituted or unsubstituted C₁-C₆ alkyl, substituted or unsubstituted C₁-C₆ fluoroalkyl, substituted or unsubstituted aryl, substituted or unsubstituted monocyclic heteroaryl, —OR⁵, —N(R⁵)₂, —CH₂OR⁵, —CN, —C(═O)OR⁵, —OC(═O)R⁵, —C(═O)N(R⁵)₂, or —NR⁵C(═O)R⁵;
each R⁵ is independently H, D, F, substituted or unsubstituted C₁-C₆ alkyl, substituted or unsubstituted C₁-C₆ fluoroalkyl, substituted or unsubstituted C₁-C₆ heteroalkyl, substituted or unsubstituted aryl, or substituted or unsubstituted heteroaryl; and
n is 1, 2, 3, 4, or 5;
with the provision that when —W—X—V— is —NHC(═O)CH₂—, then R¹ is not 2-chlorophenyl.

In some embodiments, U is N.

In some embodiments, V is —O—, —S—, —S(═O)—, —S(═O)₂—, or —CH₂—. In some embodiments, V is —O—. In some embodiments, V is —S—. In some embodiments, V is —S(═O)—.

In some embodiments, V is —S(═O)₂—. In some embodiments, V is —CH₂—.

In some embodiments, R¹ is substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, and substituted or unsubstituted heteroaryl. In some embodiments, R¹ is substituted or unsubstituted C₁-C₆ alkyl. In some embodiments, R¹ is substituted or unsubstituted C₁-C₆ fluoroalkyl. In some embodiments, R¹ is substituted or unsubstituted C₁-C₆ heteroalkyl. In some embodiments, R¹ is substituted or unsubstituted C₃-C₁₀ cycloalkyl. In some embodiments, R¹ is substituted or unsubstituted C₂-C₉ heterocycloalkyl. In some embodiments, R¹ is substituted or unsubstituted aryl. In some embodiments, R¹ is substituted or unsubstituted heteroaryl.

In some embodiments, R¹ is substituted or unsubstituted heteroaryl. In some embodiments, the heteroaryl is a bicyclic heteroaryl. In some embodiments, R¹ is substituted or unsubstituted bicyclic heteroaryl selected from the group consisting of indolinyl, indolin-2-onyl, indolin-3-onyl, indolyl, benzofuranyl, benzothiophenyl, benzimidazolyl, benzoxazole, benzothiazole, indazolyl, benzotriazolyl, indolizinyl, pyrazolo[1,5-a]pyridinyl, imidazo[1,5-a]pyridinyl, imidazo[1,2-a]pyridinyl, 2,3-dihydrobenzo[b][1,4]dioxinyl, 1,2,3,4-tetrahydroquinoxaline, 3,4-dihydro-2H-benzo[b][1,4]oxazinyl, 2,3-dihydrobenzo[b][1,4]oxathiine, 2H-benzo[b][1,4]oxazine, 2H-benzo[b][1,4]thiazine, 3,4-dihydroquinolinyl, quinolinyl, 3,4-dihydroisoquinoline, isoquinolinyl, quinazolinyl, quinoxalinyl, cinnolinyl, phthalazinyl.

In some embodiments, R¹ is substituted or unsubstituted bicyclic heteroaryl selected from the group consisting of:

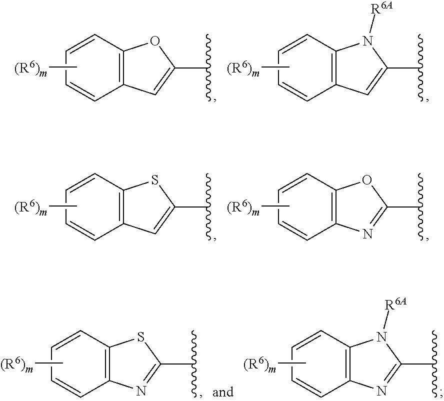

wherein each R⁶ is independently H, D, halogen, substituted or unsubstituted C₁-C₄ alkyl, substituted or unsubstituted C₁-C₄ fluoroalkyl, or substituted or unsubstituted C₁-C₄ heteroalkyl;
R^{6A} is H, D, substituted or unsubstituted C₁-C₄ alkyl, substituted or unsubstituted C₁-C₄ fluoroalkyl, or substituted or unsubstituted C₁-C₄ heteroalkyl; and
m is 1, 2, 3, 4, or 5.

In some embodiments, R⁶ is H. In some embodiments, R⁶ is D. In some embodiments, R⁶ is halogen. In some embodiments, R⁶ is substituted or unsubstituted C₁-C₄ alkyl. In some embodiments, R⁶ is substituted or unsubstituted C₁-C₄ fluoroalkyl. In some embodiments, R⁶ is substituted or unsubstituted C₁-C₄ heteroalkyl.

In some embodiments, m is 1, 2, or 3. In some embodiments, m is 1 or 2. In some embodiments, m is 1. In some embodiments, m is 2. In some embodiments, m is 3. In some embodiments, m is 4. In some embodiments, m is 5.

In some embodiments, T is —O—, —S—, —S(═O)—, —S(═O)₂—, or —(CH₂)ₙ—. In some embodiments, T is —O—. In some embodiments, T is —S—. In some embodiments, T is —S(═O)—. In some embodiments, T is —S(═O)₂—. In some embodiments, T is —(CH₂)ₙ—.

In some embodiments, n is 1, 2, 3, or 4. In some embodiments, n is 1, 2, or 3. In some embodiments, n is 2, 3, or 4. In some embodiments, n is 1 or 2. In some embodiments, n is 1. In some embodiments, n is 2. In some embodiments, n is 3. In some embodiments, n is 4. In some embodiments, n is 5.

In some embodiments, R³ is H, D, F, substituted or unsubstituted C₁-C₆ alkyl, substituted or unsubstituted C₁-C₆ fluoroalkyl, substituted or unsubstituted C₁-C₆ heteroalkyl, or substituted or unsubstituted C₃-C₁₀ cycloalkyl. In some embodiments, R³ is H. In some embodiments, R³ is D. In some embodiments, R³ is F. In some embodiments, R³ is substituted or unsubstituted C₁-C₆ alkyl. In some embodiments, R³ is substituted or unsubstituted C₁-C₆ fluoroalkyl. In some embodiments, R³ is substituted or unsubstituted C₁-C₆ heteroalkyl. In some embodiments, R³ is substituted or unsubstituted C₃-C₁₀ cycloalkyl.

In some embodiments, Y is —C(═O)NR⁴—, —NR⁴C(═O)—, —OC(═O)NR⁴—, —NR⁴C(═O)O—, —NR⁴C(═O)NR⁴—, —S(═O)₂NR⁴—, or —NR⁴S(═O)₂—. In some embodiments, Y is —C(═O)NR⁴—. In some embodiments, Y is —NR⁴C(═O)—. In some embodiments, Y is —OC(═O)NR⁴—. In some embodiments, Y is —NR⁴C(═O)O—. In some embodiments, Y is —NR⁴C(═O)NR⁴—. In some embodiments, Y is —S(═O)₂NR⁴—. In some embodiments, Y is —NR⁴S(═O)₂—.

In some embodiments, Y is —C(═O)NH—, —NHC(═O)—, —OC(═O)NH—, —NHC(═O)O—, —NHC(═O)NH—, —S(═O)₂NH—, or —NHS(═O)₂—. In some embodiments, Y is —C(═O)NH—. In some embodiments, Y is —NHC(═O)—. In some embodiments, Y is —OC(═O)NH—. In some embodiments, Y is —NHC(═O)O—. In some embodiments, Y is —NHC(═O)NH—. In some embodiments, Y is —S(═O)₂NH—. In some embodiments, Y is —NHS(═O)₂—.

In some embodiments, —Y—Z—R² is in exo-position. In some embodiments, —Y—Z—R² is in endo-position.

In some embodiments, R² is substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, and substituted or unsubstituted heteroaryl. In some embodiments, R² is substituted or unsubstituted C₁-C₆ alkyl. In some embodiments, R² is substituted or unsubstituted C₁-C₆ fluoroalkyl. In some embodiments, R² is substituted or unsubstituted C₁-C₆ heteroalkyl. In some embodiments, R² is substituted or unsubstituted C₃-C₁₀ cycloalkyl. In some embodiments, R² is substituted or unsubstituted C₂-C₉ heterocycloalkyl. In some embodiments, R² is substituted or unsubstituted aryl. In some embodiments, R² is substituted or unsubstituted heteroaryl.

In some embodiments, R² is

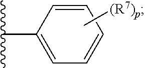

wherein each R⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, or substituted or unsubstituted C₂-C₉ heterocycloalkyl. In some embodiments, each R⁷ is independently substituted or unsubstituted C₁-C₆ alkyl; and p is 1, 2, 3, 4, or 5.

In some embodiments, each R⁷ is independently substituted or unsubstituted C₁-C₆ alkyl. In some embodiments, each R⁷ is independently substituted or unsubstituted C₁-C₆ fluoroalkyl. In some embodiments, each R⁷ is independently substituted or unsubstituted C₁-C₆ heteroalkyl. In some embodiments, each R⁷ is independently substituted or unsubstituted C₃-C₁₀ cycloalkyl. In some embodiments, each R⁷ is independently substituted or unsubstituted C₂-C₉ heterocycloalkyl. In some embodiments, each R⁷ is independently selected from the group consisting of -OMe, -OEt, -On-Pr, -Oi-Pr, -On-butyl, -Osec-butyl, and -Oi-butyl.

In some embodiments, p is 1, 2, or 3. In some embodiments, p is 1 or 2. In some embodiments, p is 2 or 3. In some embodiments, p is 1. In some embodiments, p is 2. In some embodiments, p is 3. In some embodiments, p is 4. In some embodiments, p is 5.

In some embodiments, the compound of Formula (I) has the structure of Formula (Ia), or a pharmaceutically acceptable salt, solvate, or N-oxide thereof:

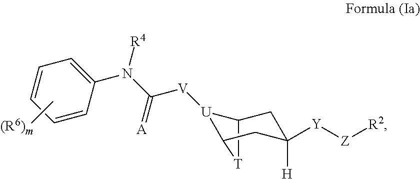

wherein A is O, S, or NR⁴.

In some embodiments, the compound of Formula (I) has the structure of Formula (Ib), or a pharmaceutically acceptable salt, solvate, prodrug, or N-oxide thereof:

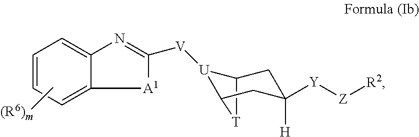

wherein A¹ is O, S, or NR^{6A}.

In some embodiments, the compound of Formula (I) has the structure of Formula (Ic), or a pharmaceutically acceptable salt, solvate, prodrug, or N-oxide thereof:

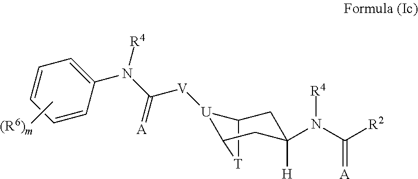

wherein each A is independently O, S, or NR⁴.

In some embodiments, the compound of Formula (I) has the structure of Formula (Id), or a pharmaceutically acceptable salt, solvate, prodrug, or N-oxide thereof:

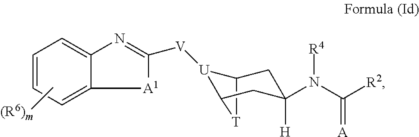

wherein A is O, S, or NR⁴ and A¹ is O, S, or NR^{6A}.

In another aspect, described herein is a CXCR6 receptor inhibitor that has the structure of Formula (II), or a pharmaceutically acceptable salt, solvate, or N-oxide thereof:

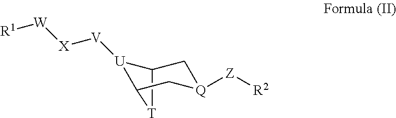

wherein,
U and Q are independently N or CR⁴;
W, X, V, T, and Z are independently absent, —O—, —S—, —S(═O)—, —S(═O)₂—, —(CR⁴R⁴)ₙ—, —C(═O)—, —C(═NR⁴)—, —C(═O)O—, —OC(═O)—, —C(═O)C(═O)—, —C(═O)NR⁴—, —NR⁴C(═O)—, —OC(═O)NR⁴—, —NR⁴C(═O)O—, —NR⁴C(═O)NR⁴—, —S(═O)₂NR⁴—, —NR⁴S(═O)₂—, —NR⁴—;
R¹ and R² are independently selected from the group consisting of H, D, halogen, —CN, —OR⁴, —SR⁴, —S(═O)R⁴, —S(═O)₂R⁴, —N(R⁴)₂, —NR⁴S(═O)(═NR⁴)R⁴, —NR⁴S(═O)₂R⁴, —S(═O)₂N(R⁴)₂, —C(═O)R⁴, —OC(═O)R⁴, —C(═O)OR⁵, —OC(═O)OR⁵, —C(═O)N(R⁴)₂, —OC(═O)N(R⁴)₂, —NR⁴C(═O)R⁴, substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, and substituted or unsubstituted heteroaryl;
each R⁴ is independently H, D, substituted or unsubstituted C₁-C₆ alkyl, substituted or unsubstituted C₁-C₆ fluoroalkyl, substituted or unsubstituted aryl, substituted or unsubstituted monocyclic heteroaryl, —OR⁵, —N(R⁵)₂, —CH₂OR⁵, —CN, —C(═O)OR⁵, —OC(═O)R⁵, —C(═O)N(R⁵)₂, or —NR⁵C(═O)R⁵;
each R⁵ is independently H, D, F, substituted or unsubstituted C₁-C₆ alkyl, substituted or unsubstituted C₁-C₆ fluoroalkyl, substituted or unsubstituted C₁-C₆ heteroalkyl, substituted or unsubstituted aryl, or substituted or unsubstituted heteroaryl; and
n is 1, 2, 3, 4, or 5;
with the provision that when —W—X—V— is —NHC(═O)CH₂—, then R¹ is not 2-chlorophenyl.

In some embodiments, U is N.

In some embodiments, V is —O—, —S—, —S(═O)—, —S(═O)₂—, or —CH₂—. In some embodiments, V is —O—. In some embodiments, V is —S—. In some embodiments, V is —S(═O)—. In some embodiments, V is —S(═O)₂—. In some embodiments, V is —CH₂—.

In some embodiments, R¹ is substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, and substituted or unsubstituted heteroaryl. In some embodiments, R¹ is substituted or unsubstituted C₁-C₆ alkyl. In some embodiments, R¹ is substituted or unsubstituted C₁-C₆ fluoroalkyl. In some embodiments, R¹ is substituted or unsubstituted C₁-C₆ heteroalkyl. In some embodiments, R¹ is substituted or unsubstituted C₃-C₁₀ cycloalkyl. In some embodiments, R¹ is substituted or unsubstituted C₂-C₉ heterocycloalkyl. In some embodiments, R¹ is substituted or unsubstituted aryl. In some embodiments, R¹ is substituted or unsubstituted heteroaryl.

In some embodiments, R¹ is substituted or unsubstituted heteroaryl. In some embodiments, the heteroaryl is a bicyclic heteroaryl. In some embodiments, R¹ is substituted or unsubstituted bicyclic heteroaryl selected from the group consisting of indolinyl, indolin-2-onyl, indolin-3-onyl, indolyl, benzofuranyl, benzothiophenyl, benzimidazolyl, benzoxazole, benzothiazole, indazolyl, benzotriazolyl, indolizinyl, pyrazolo[1,5-a]pyridinyl, imidazo[1,5-a]pyridinyl, imidazo[1,2-a]pyridinyl, 2,3-dihydrobenzo[b][1,4]dioxinyl, 1,2,3,4-tetrahydroquinoxaline, 3,4-dihydro-2H-benzo[b][1,4]oxazinyl, 2,3-dihydrobenzo[b][1,4]oxathiine, 2H-benzo[b][1,4]oxazine, 2H-benzo[b][1,4]thiazine, 3,4-dihydroquinolinyl, quinolinyl, 3,4-dihydroisoquinoline, isoquinolinyl, quinazolinyl, quinoxalinyl, cinnolinyl, phthalazinyl.

In some embodiments, R¹ is substituted or unsubstituted bicyclic heteroaryl selected from the group consisting of:

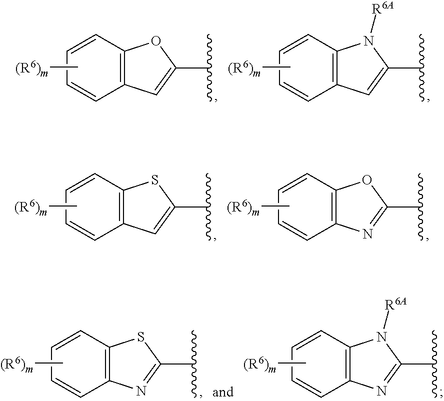

wherein each R⁶ is independently H, D, halogen, substituted or unsubstituted C₁-C₄ alkyl, substituted or unsubstituted C₁-C₄ fluoroalkyl, or substituted or unsubstituted C₁-C₄ heteroalkyl;
R^{6A} is H, D, substituted or unsubstituted C₁-C₄ alkyl, substituted or unsubstituted C₁-C₄ fluoroalkyl, or substituted or unsubstituted C₁-C₄ heteroalkyl; and
m is 1, 2, 3, 4, or 5.

In some embodiments, R⁶ is H. In some embodiments, R⁶ is D. In some embodiments, R⁶ is halogen. In some embodiments, R⁶ is substituted or unsubstituted C₁-C₄ alkyl. In some embodiments, R⁶ is substituted or unsubstituted C₁-C₄ fluoroalkyl. In some embodiments, R⁶ is substituted or unsubstituted C₁-C₄ heteroalkyl.

In some embodiments, m is 1, 2, or 3. In some embodiments, m is 1 or 2. In some embodiments, m is 1. In some embodiments, m is 2. In some embodiments, m is 3. In some embodiments, m is 4. In some embodiments, m is 5.

In some embodiments, T is —O—, —S—, —S(═O)—, —S(═O)₂—, or —(CH₂)ₙ—. In some embodiments, T is —O—. In some embodiments, T is —S—. In some embodiments, T is —S(═O)—. In some embodiments, T is —S(═O)₂—. In some embodiments, T is —(CH₂)ₙ—.

In some embodiments, n is 1, 2, or 3. In some embodiments, n is 2, 3, or 4. In some embodiments, n is 1 or 2. In some embodiments, n is 1. In some embodiments, n is 2. In some embodiments, n is 3. In some embodiments, n is 4. In some embodiments, n is 5.

In some embodiments, Q is N or CR⁴. In some embodiments, Q is N. In some embodiments, Q is CR⁴.

In some embodiments, the compound of Formula (II) has the structure of Formula (IIa), or a pharmaceutically acceptable salt, solvate, prodrug, or N-oxide thereof:

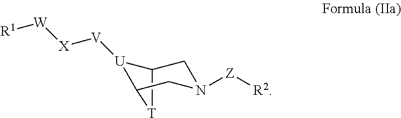

In some embodiments, Z is —C(═O)NR⁴—, —NR⁴C(═O)—, —OC(═O)NR⁴—, —NR⁴C(═O)O—, —NR⁴C(═O)NR⁴—, —S(═O)₂NR⁴—, or —NR⁴S(═O)₂—. In some embodiments, Z is —C(═O)NR⁴—. In some embodiments, Z is —NR⁴C(═O)—. In some embodiments, Z is —OC(═O)NR⁴—. In some embodiments, Z is —NR⁴C(═O)O—. In some embodiments, Z is —NR⁴C(═O)NR⁴—. In some embodiments, Z is —S(═O)₂NR⁴—. In some embodiments, Z is —NR⁴S(═O)₂—.

In some embodiments, Z is —C(═O)NH—, —NHC(═O)—, —OC(═O)NH—, —NHC(═O)O—, —NHC(═O)NH—, —S(═O)₂NH—, or —NHS(═O)₂—. In some embodiments, Z is —C(═O)NH—. In some embodiments, Z is —NHC(═O)—. In some embodiments, Z is —OC(═O)NH—. In some embodiments, Z is —NHC(═O)O—. In some embodiments, Z is —NHC(═O)NH—. In some embodiments, Z is —S(═O)₂NH—. In some embodiments, Z is —NHS(═O)₂—.

In some embodiments, —Z—R² is in exo-position. In some embodiments, —Z—R² is in endo-position.

In some embodiments, R² is substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, and substituted or unsubstituted heteroaryl. In some embodiments, R² is substituted or unsubstituted C₁-C₆ alkyl. In some embodiments, R² is substituted or unsubstituted C₁-C₆ fluoroalkyl. In some embodiments, R² is substituted or unsubstituted C₁-C₆ heteroalkyl. In some embodiments, R² is substituted or unsubstituted C₃-C₁₀ cycloalkyl. In some embodiments, R² is substituted or unsubstituted C₂-C₉ heterocycloalkyl. In some embodiments, R² is substituted or unsubstituted aryl. In some embodiments, R² is substituted or unsubstituted heteroaryl.

In some embodiments, R² is

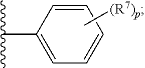

wherein each R⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, or substituted or unsubstituted C₂-C₉ heterocycloalkyl. In some embodiments, each R⁷ is independently substituted or unsubstituted C₁-C₆ alkyl; and p is 1, 2, 3, 4, or 5.

In some embodiments, each R⁷ is independently substituted or unsubstituted C₁-C₆ fluoroalkyl. In some embodiments, each R⁷ is independently substituted or unsubstituted C₁-C₆ heteroalkyl. In some embodiments, each R⁷ is independently substituted or unsubstituted C₃-C₁₀ cycloalkyl. In some embodiments, each R⁷ is independently substituted or unsubstituted C₂-C₉ heterocycloalkyl. In some embodiments, each R⁷ is independently selected from the group consisting of -OMe, -OEt, -On-Pr, -Oi-Pr, -On-butyl, -Osec-butyl, and -Oi-butyl.

In some embodiments, p is 1, 2, or 3. In some embodiments, p is 1 or 2. In some embodiments, p is 2 or 3. In some embodiments, p is 1. In some embodiments, p is 2. In some embodiments, p is 3. In some embodiments, p is 4. In some embodiments, p is 5.

In some embodiments, compounds described herein have the following structure:

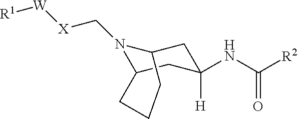

or a pharmaceutically acceptable salt, solvate, prodrug, or N-oxide thereof.

In some embodiments, X is absent, —C(═O)—, or —C(═NR⁴)—. In some embodiments, X is —C(═O)—, or —C(═NR⁴)—. In some embodiments, X is —C(═O)—.

In some embodiments, W is absent, —O—, —S—, —(CR⁴R⁴)ₙ—, —NR⁴—. In some embodiments, W is absent, or —NR⁴—. In some embodiments, W is —NH—. In some embodiments, W is absent.

In some embodiments, X is absent, —C(═O)—, or —C(═NR⁴)—; and W is absent, —O—, —S—, —(CR⁴R⁴)ₙ—, —NR⁴—. In some embodiments, X is absent, or —C(═O)—; and W is absent, or —NR⁴—. In some embodiments, X is absent, or —C(═O)—; and W is —NR⁴—. In some embodiments, X is —C(═O)—; and W is —NH—. In some embodiments, X is absent; and W is absent.

In some embodiments, V is absent, or —CH₂—. In some embodiments, V is —CH₂—.

In some embodiments, X is absent, —C(═O)—, or —C(═NR⁴)—; W is absent, —O—, —S—, —(CR⁴R⁴)ₙ—, —NR⁴—; and V is absent, or —CH₂—. In some embodiments, X is absent, or —C(═O)—; W is absent, or —NR⁴—; and V is —CH₂—. In some embodiments, X is absent; W is —NH—; and V is —CH₂—. In some embodiments, X is —C(═O)—; W is —NR⁴—; and V is —CH₂—. In some embodiments, X is —C(═O)—; W is absent; and V is —CH₂—.

In some embodiments, X is absent, —C(═O)—, or —C(═NR⁴)—; W is absent, —O—, —S—, —(CR⁴R⁴)ₙ—, —NR⁴—; and V is absent, or —CH₂—. In some embodiments, X is absent, —C(═O)—; and W is absent, or —NR⁴—. In some embodiments, X is absent; W is absent; and V is absent, or —CH₂—. In some embodiments, X is absent; W is absent; and V is absent. In some embodiments, X is absent; W is absent; and V is —CH₂—.

In some embodiments, —W—X— is absent or —NHC(=A)-; A is O, S, or NR⁴. In some embodiments, A is O. In some embodiments, —W—X— is —NHC(═O). In some embodiment, —W—X— is absent.

In some embodiments, —W—X—V— is —NHC(═O)CH₂—. In some embodiments, —W—X—V— is —CH₂—.

In some embodiments, R¹ is a substituted or unsubstituted bicyclic heteroaryl; and R² is substituted or unsubstituted aryl.

In some embodiments, R¹ is

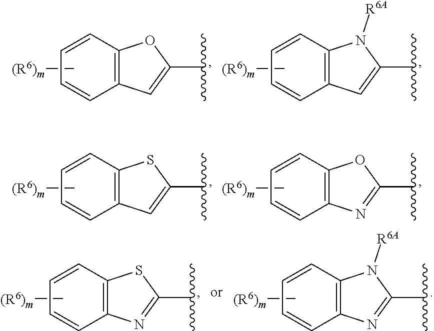

In some embodiments, each R⁶ is independently D, halogen, substituted or unsubstituted C₁-C₄ alkyl, substituted or unsubstituted C₁-C₄ fluoroalkyl, or substituted or unsubstituted C₁-C₄ heteroalkyl. In some embodiment, R^{6A} is H, D, substituted or unsubstituted C₁-C₄ alkyl, substituted or unsubstituted C₁-C₄ fluoroalkyl, or substituted or unsubstituted C₁-C₄ heteroalkyl. In some embodiments, each R⁶ is independently halogen, unsubstituted C₁-C₄ alkyl, or unsubstituted C₁-C₄ fluoroalkyl. In some embodiments, each R⁶ is independently F, C₁, or CF₃.

In some embodiments, m is 0, 1, 2, 3, 4, or 5. In some embodiments, m is 0, 1, or 2.

In some embodiments, R¹—W—X— is

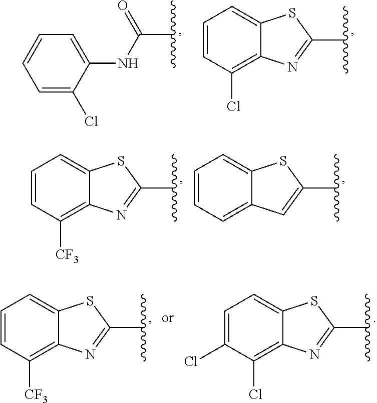

In some embodiments, R² is

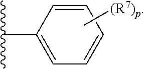

In some embodiments, each R⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, or substituted or unsubstituted C₂-C₉ heterocycloalkyl. In some embodiments, each R⁷ is independently unsubstituted C₁-C₆ alkyl, unsubstituted C₁-C₆ fluoroalkyl, or unsubstituted C₁-C₆ heteroalkyl. In some embodiments, each R⁷ is independently C₁-C₆ alkoxy. In some embodiments, each R⁷ is independently methoxy, ethoxy, isopropoxy, sec-butoxy, or tert-butoxy. In some embodiments, each R⁷ is independently methoxy. In some embodiments, at least one R⁷ is methoxy.

In some embodiments, p is 0, 1, 2, 3, 4, or 5. In some embodiments, p is 1, 2, or 3. In some embodiments, p is 1, or 2.

In some embodiments, R² is

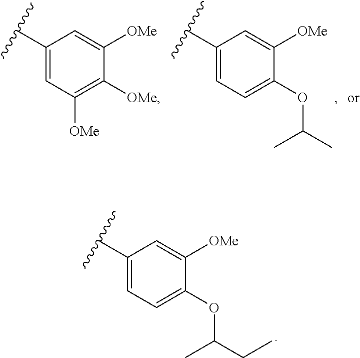 [SMILES: *c1cc(OC)c(OC)c(OC)c1 ; *c1ccc(OC(C)C)c(OC)c1 ; *c1ccc(OC(C)CC)c(OC)c1]

In some embodiments, compounds described herein have the following structure:

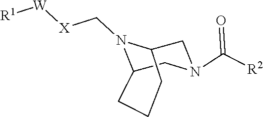

or a pharmaceutically acceptable salt, solvate, prodrug, or N-oxide thereof.

In some embodiments, X is absent, —C(═O)—, or —C(═NR⁴)—. In some embodiments, X is —C(═O)—, or —C(═NR⁴)—. In some embodiments, X is —C(═O)—.

In some embodiments, W is absent, —O—, —S—, —(CR⁴R⁴)ₙ—, —NR⁴—. In some embodiments, W is absent, or —NR⁴—. In some embodiments, W is —NH—. In some embodiments, W is absent.

In some embodiments, X is absent, —C(═O)—, or —C(═NR⁴)—; and W is absent, —O—, —S—, —(CR⁴R⁴)ₙ—, —NR⁴—. In some embodiments, X is absent, or —C(═O)—; and W is absent, or —NR⁴—. In some embodiments, X is absent, or —C(═O)—; and W is —NR⁴—. In some embodiments, X is —C(═O)—; and W is —NH—. In some embodiments, X is absent; and W is absent.

In some embodiments, V is absent, or —CH₂—. In some embodiments, V is —CH₂—.

In some embodiments, X is absent, —C(═O)—, or —C(═NR⁴)—; W is absent, —O—, —S—, —(CR⁴R⁴)ₙ—, —NR⁴—; and V is absent, or —CH₂—. In some embodiments, X is absent, or —C(═O)—; W is absent, or —NR⁴—; and V is —CH₂—. In some embodiments, X is absent; W is —NH—; and V is —CH₂—. In some embodiments, X is —C(═O)—; W is —NR⁴—; and V is —CH₂—. In some embodiments, X is —C(═O)—; W is absent; and V is —CH₂—.

In some embodiments, X is absent, —C(═O)—, or —C(═NR⁴)—; W is absent, —O—, —S—, —(CR⁴R⁴)ₙ—, —NR⁴—; and V is absent, or —CH₂—. In some embodiments, X is absent, —C(═O)—; and W is absent, or —NR⁴—. In some embodiments, X is absent; W is absent; and V is absent, or —CH₂—. In some embodiments, X is absent; W is absent; and V is absent. In some embodiments, X is absent; W is absent; and V is —CH₂—.

In some embodiments, —W—X— is absent or —NHC(=A)-; A is O, S, or NR⁴. In some embodiments, A is O. In some embodiments, —W—X— is —NHC(═O). In some embodiment, —W—X— is absent.

In some embodiments, —W—X—V— is —NHC(═O)CH₂—. In some embodiments, —W—X—V— is —CH₂—.

In some embodiments, R¹ is a substituted or unsubstituted bicyclic heteroaryl; and R² is substituted or unsubstituted aryl.

In some embodiments, R¹ is

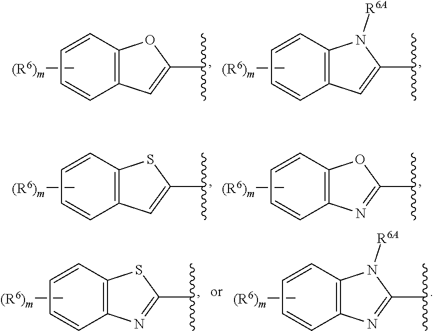

In some embodiments, each R⁶ is independently D, halogen, substituted or unsubstituted C₁-C₄alkyl, substituted or unsubstituted C₁-C₄fluoroalkyl, or substituted or unsubstituted C₁-C₄heteroalkyl. In some embodiment, R^{6A} is H, D, substituted or unsubstituted C₁-C₄alkyl, substituted or unsubstituted C₁-C₄fluoroalkyl, or substituted or unsubstituted C₁-C₄heteroalkyl. In some embodiments, each R⁶ is independently halogen, unsubstituted C₁-C₄alkyl, or unsubstituted C₁-C₄fluoroalkyl. In some embodiments, each R⁶ is independently F, Cl, or CF₃.

In some embodiments, m is 0, 1,2, 3, 4, or 5. In some embodiments, m is 0, 1, or 2.

In some embodiments, R¹—W—X is

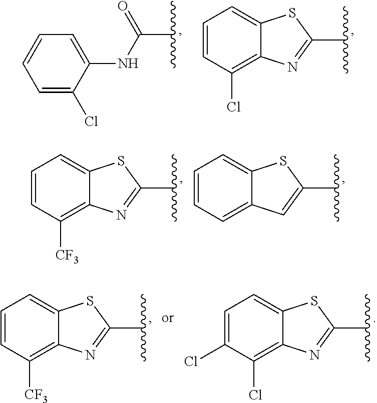

In some embodiments, R² is

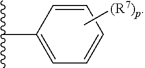

In some embodiments, each R⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, or substituted or unsubstituted C₂-C₉ heterocycloalkyl. In some embodiments, each R⁷ is independently unsubstituted C₁-C₆ alkyl, unsubstituted C₁-C₆ fluoroalkyl, or unsubstituted C₁-C₆ heteroalkyl. In some embodiments, each R⁷ is independently C₁-C₆ alkoxy. In some embodiments, each R⁷ is independently methoxy, ethoxy, isopropoxy, sec-butoxy, or tert-butoxy. In some embodiments, each R⁷ is independently methoxy. In some embodiments, at least one R⁷ is methoxy.

In some embodiments, p is 0, 1, 2, 3, 4, or 5. In some embodiments, p is 1, 2, or 3. In some embodiments, p is 1 or 2.

In some embodiments, R² is

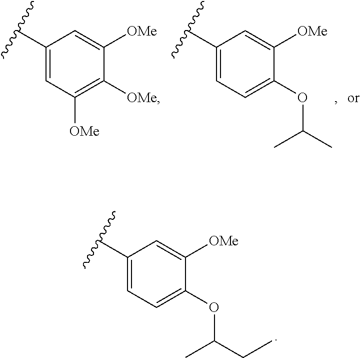 [SMILES: *c1cc(OC)c(OC)c(OC)c1 ; *c1ccc(OC(C)C)c(OC)c1 ; *c1ccc(OC(C)CC)c(OC)c1]

Any combination of the groups described above for the various variables is contemplated herein. Throughout the specification, groups and substituents thereof are chosen by one skilled in the field to provide stable moieties and compounds.

Exemplary compounds include the compounds described in the following Tables:

TABLE 1

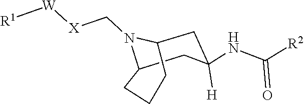

| R¹—W—X | R² | Compound Name |
|---|---|---|
| 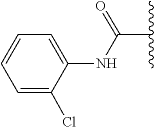 [SMILES: *C(=O)Nc1ccccc1Cl] | 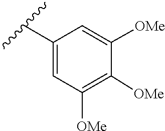 [SMILES: *c1cc(OC)c(OC)c(OC)c1] | N-((1R,3s,5S)-9-(2-((2-chlorophenyl)amino)-2- oxoethyl)-9-azabicyclo[3.3.1]nonan-3-yl)-3,4,5- trimethoxybenzamide |
| 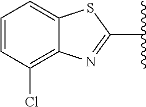 [SMILES: *c1nc2c(Cl)cccc2s1] | 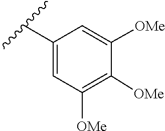 [SMILES: *c1cc(OC)c(OC)c(OC)c1] | N-((1R,3s,5S)-9-((4-chlorobenzo[d]thiazol-2- yl)methyl)-9-azabicyclo[3.3.1]nonan-3-yl)- 3,4,5-trimethoxybenzamide |
| 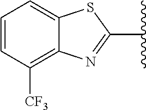 [SMILES: *c1nc2c(C(F)(F)F)cccc2s1] | 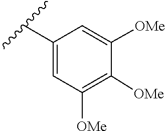 [SMILES: *c1cc(OC)c(OC)c(OC)c1] | 3,4,5-trimethoxy-N-((1R,3s,5S)-9-((4- (trifluoromethyl)benzo[d]thiazol-2-yl)methyl)- 9-azabicyclo[3.3.1]nonan-3-yl)benzamide (Compound 1) |
| 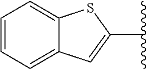 [SMILES: *c1cc2ccccc2s1] | 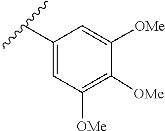 [SMILES: *c1cc(OC)c(OC)c(OC)c1] | N-((1R,3s,5S)-9-(benzo[b]thiophen-2- ylmethyl)-9-azabicyclo[3.3.1]nonan-3-yl)- 3,4,5-trimethoxybenzamide |
| 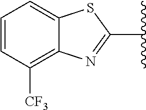 [SMILES: *c1nc2c(C(F)(F)F)cccc2s1] | 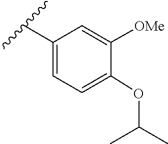 [SMILES: *c1ccc(OC(C)C)c(OC)c1] | 4-isopropoxy-3-methoxy-N-((1R,3s,5S)-9-((4- (trifluoromethyl)benzo[d]thiazol-2-yl)methyl)- 9-azabicyclo[3.3.1]nonan-3-yl)benzamide |
| 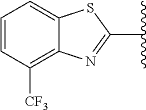 [SMILES: *c1nc2c(C(F)(F)F)cccc2s1] | 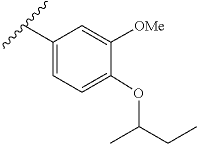 [SMILES: *c1ccc(OC(C)CC)c(OC)c1] | 4-(sec-butoxy)-3-methoxy-N-((1R,3s,5S)-9-((4- (trifluoromethyl)benzo[d]thiazol-2-yl)methyl)- 9-azabicyclo[3.3.1]nonan-3-yl)benzamide |
| 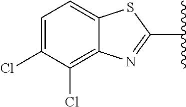 [SMILES: *c1nc2c(Cl)c(Cl)ccc2s1] | 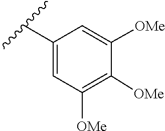 [SMILES: *c1cc(OC)c(OC)c(OC)c1] | N-((1R,3s,5S)-9-((4,5-dichlorobenzo[d]thiazol- 2-yl)methyl)-9-azabicyclo[3.3.1]nonan-3-yl)- 3,4,5-trimethoxybenzamide |

In one aspect, provided herein is a pharmaceutically acceptable salt, solvate, or N-oxide thereof of a compound described in Table 1.

TABLE 2

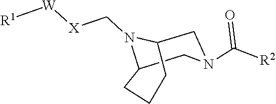

| R¹—W—X | R² | Compound Name |
|---|---|---|
| 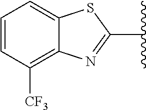 [SMILES: *c1nc2c(C(F)(F)F)cccc2s1] | 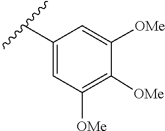 [SMILES: *c1cc(OC)c(OC)c(OC)c1] | ((1R,5S)-9-((4- (trifluoromethyl)benzo[d]thiazol-2- yl)methyl)-3,9-diazabicyclo[3.3.1]nonan-3- yl)(3,4,5-trimethoxyphenyl)methanone |

In one aspect, provided herein is a pharmaceutically acceptable salt, solvate, or N-oxide thereof of a compound described in Table 2.

In some embodiments, a CXCR6 receptor inhibitor is selected from the group consisting of:
N-phenyl-2-{4-[(3,4,5-trimethoxyphenyl)carbonylamino]piperidyl}acetamide;
N-phenyl-2-{3-[(3,4,5-trimethoxyphenyl)carbonylamino]-9-azabicyclo[3.3.1]non-9-yl}acetamide;
N-phenyl-2-{3-[(3,4,5-trimethoxyphenyl)carbonylamino]-8-azabicyclo[3.2.1]oct-8-yl}acetamide;
N-phenyl-2-{3-[(3,4,5-trimethoxyphenyl)carbonylamino]-8-azabicyclo[3.2.1]oct-8-yl}acetamide;
2-{3-[(4-methoxyphenyl)carbonylamino]-9-azabicyclo[3.3.1]non-9-yl}-N-(4-methylphenyl)acetamide;
2-{3-[(4-methoxyphenyl)carbonylamino]-9-azabicyclo[3.3.1]non-9-yl}-N-phenylacetamide;
N-(4-fluorophenyl)-2-{3-[(4-methoxyphenyl)carbonylamino]-9-azabicyclo[3.3.1]non-9-yl}acetamide;
N-(4-chlorophenyl)-2-{3-[(4-methoxyphenyl)carbonylamino]-9-azabicyclo[3.3.1]non-9-yl}acetamide;
N-(4-methoxyphenyl)-2-{3-[(4-methoxyphenyl)carbonylamino]-9-azabicyclo[3.3.1]non-9-yl}acetamide;
2-{3-[(3,4-dimethoxyphenyl)carbonylamino]-9-azabicyclo[3.3.1]non-9-yl}-N-phenylacetamide;
2-{3-[(3,4-dimethoxyphenyl)carbonylamino]-9-azabicyclo[3.3.1]non-9-yl}-N-(4-fluorophenyl)acetamide;
2-{3-[(3,4-dimethoxyphenyl)carbonylamino]-9-azabicyclo[3.3.1]non-9-yl}-N-(4-chlorophenyl)acetamide;
2-{3-[(3,4-dimethoxyphenyl)carbonylamino]-9-azabicyclo[3.3.1]non-9-yl}-N-(4-methylphenyl)acetamide;
2-{3-[(3,5-dimethoxyphenyl)carbonylamino]-9-azabicyclo[3.3.1]non-9-yl}-N-phenylacetamide;
2-{3-[(3,5-dimethoxyphenyl)carbonylamino]-9-azabicyclo[3.3.1]non-9-yl}-N-(4-fluorophenyl)acetamide;
2-{3-[(3,5-dimethoxyphenyl)carbonylamino]-9-azabicyclo[3.3.1]non-9-yl}-N-(4-chlorophenyl)acetamide;
2-{3-[(3,5-dimethoxyphenyl)carbonylamino]-9-azabicyclo[3.3.1]non-9-yl}-N-(4-methylphenyl)acetamide;
2-{3-[(3,5-dimethoxyphenyl)carbonylamino]-9-azabicyclo[3.3.1]non-9-yl}-N-(4-methoxyphenyl)acetamide;
2-{3-[(3,4-dimethoxyphenyl)carbonylamino]-9-azabicyclo[3.3.1]non-9-yl}-N-(4-methoxyphenyl)acetamide;
N-(4-fluorophenyl)-2-{3-[(3,4,5-trimethoxyphenyl)carbonylamino]-9-azabicyclo[3.3.1]non-9-yl}acetamide;
N-(4-chlorophenyl)-2-{3-[(3,4,5-trimethoxyphenyl)carbonylamino]-9-azabicyclo[3.3.1]non-9-yl}acetamide;
N-(4-methylphenyl)-2-{3-[(3,4,5-trimethoxyphenyl)carbonylamino]-9-azabicyclo[3.3.1]non-9-yl}acetamide;
N-(4-methoxyphenyl)-2-{3-[(3,4,5-trimethoxyphenyl)carbonylamino]-9-azabicyclo[3.3.1]non-9-yl}acetamide;
N-(2-methoxyphenyl)-2-{3-[(3,4,5-trimethoxyphenyl)carbonylamino]-9-azabicyclo[3.3.1]non-9-yl}acetamide;
N-(2-chlorophenyl)-2-{3-[(3,4,5-trimethoxyphenyl)carbonylamino]-9-azabicyclo[3.3.1]non-9-yl}acetamide;
2-{3-[(3-methoxyphenyl)carbonylamino]-9-azabicyclo[3.3.1]non-9-yl}-N-phenylacetamide;
N-(4-fluorophenyl)-2-{3-[(3-methoxyphenyl)carbonylamino]-9-azabicyclo[3.3.1]non-9-yl}acetamide;
N-(4-chlorophenyl)-2-{3-[(3-methoxyphenyl)carbonylamino]-9-azabicyclo[3.3.1]non-9-yl}acetamide;
2-{3-[(3-methoxyphenyl)carbonylamino]-9-azabicyclo[3.3.1]non-9-yl}-N-(4-methylphenyl)acetamide;
N-(4-methoxyphenyl)-2-{3-[(3-methoxyphenyl)carbonylamino]-9-azabicyclo[3.3.1]non-9-yl}acetamide;
N-{9-[(4-methoxyphenyl)methyl]-9-azabicyclo[3.3.1]non-3-yl}(3,4,5-trimethoxyphenyl)carboxamide;
2-[3-(2H-benzo[d]1,3-dioxolan-5-ylcarbonylamino)-9-azabicyclo[3.3.1]non-9-yl]-N-phenylacetamide;
2-[3-(2H-benzo[d]1,3-dioxolan-5-ylcarbonylamino)-9-azabicyclo[3.3.1]non-9-yl]-N-(4-methylphenyl)acetamide;
2-[3-(2H-benzo[d]1,3-dioxolan-5-ylcarbonylamino)-9-azabicyclo[3.3.1]non-9-yl]-N-(4-fluorophenyl)acetamide;
2-[3-(2H-benzo[d]1,3-dioxolan-5-ylcarbonylamino)-9-azabicyclo[3.3.1]non-9-yl]-N-(4-methoxyphenyl)acetamide;
2-[3-(2H-benzo[d]1,3-dioxolan-5-ylcarbonylamino)-9-azabicyclo[3.3.1]non-9-yl]-N-(4-chlorophenyl)acetamide;
ethyl 2-(2-{3-[(3,4,5-trimethoxyphenyl)carbonylamino]-9-azabicyclo[3.3.1]non-9-yl}acetylamino)benzoate;
N-[(3-chlorophenyl)methyl]-2-{3-[(3,4,5-trimethoxyphenyl)carbonylamino]-9-azabicyclo[3.3.1]non-9-yl}acetamide;
N-(3-methylphenyl)-2-{3-[(3,4,5-trimethoxyphenyl)carbonylamino]-9-azabicyclo[3.3.1]non-9-yl}acetamide;
N-(2,6-dichlorophenyl)-2-{3-[(3,4,5-trimethoxyphenyl)carbonylamino]-9-azabicyclo[3.3.1]non-9-yl}acetamide;
N-(3,4-dichlorophenyl)-2-{3-[(3,4,5-trimethoxyphenyl)carbonylamino]-9-azabicyclo[3.3.1]non-9-yl}acetamide;
N-(3,5-dichlorophenyl)-2-{3-[(3,4,5-trimethoxyphenyl)carbonylamino]-9-azabicyclo[3.3.1]non-9-yl}acetamide;
N-(2-bromophenyl)-2-{3-[(3,4,5-trimethoxyphenyl)carbonylamino]-9-azabicyclo[3.3.1]non-9-yl}acetamide;
N-(3-bromophenyl)-2-{3-[(3,4,5-trimethoxyphenyl)carbonylamino]-9-azabicyclo[3.3.1]non-9-yl}acetamide;
N-(2,3-dichlorophenyl)-2-{3-[(3,4,5-trimethoxyphenyl)carbonylamino]-9-azabicyclo[3.3.1]non-9-yl}acetamide;
N-[(2-chlorophenyl)methyl]-2-{3-[(3,4,5-trimethoxyphenyl)carbonylamino]-9-azabicyclo[3.3.1]non-9-yl}acetamide;
N-(3-methoxyphenyl)-2-{3-[(3,4,5-trimethoxyphenyl)carbonylamino]-9-azabicyclo[3.3.1]non-9-yl}acetamide;
N-(2,6-dimethylphenyl)-2-{3-[(3,4,5-trimethoxyphenyl)carbonylamino]-9-azabicyclo[3.3.1]non-9-yl}acetamide;
N-[2-(trifluoromethyl)phenyl]-2-{3-[(3,4,5-trimethoxyphenyl)carbonylamino]-9-azabicyclo[3.3.1]non-9-yl}acetamide;
N-(2,5-dimethylphenyl)-2-{3-[(3,4,5-trimethoxyphenyl)carbonylamino]-9-azabicyclo[3.3.1]non-9-yl}acetamide;
N-[9-(2-indolinyl-2-oxoethyl)-9-azabicyclo[3.3.1]non-3-yl](3,4,5-trimethoxyphenyl)carboxamide;
N-(2-cyanophenyl)-2-{3-[(3,4,5-trimethoxyphenyl)carbonylamino]-9-azabicyclo[3.3.1]non-9-yl}acetamide;
N-(2-methylphenyl)-2-{3-[(3,4,5-trimethoxyphenyl)carbonylamino]-9-azabicyclo[3.3.1]non-9-yl}acetamide;
N-(3-chlorophenyl)-2-{3-[(3,4,5-trimethoxyphenyl)carbonylamino]-9-azabicyclo[3.3.1]non-9-yl}acetamide;
N-(3-fluorophenyl)-2-{3-[(3,4,5-trimethoxyphenyl)carbonylamino]-9-azabicyclo[3.3.1]non-9-yl}acetamide;
N-(2,4-dichlorophenyl)-2-{3-[(3,4,5-trimethoxyphenyl)carbonylamino]-9-azabicyclo[3.3.1]non-9-yl}acetamide;
N-(2-fluorophenyl)-2-{3-[(3,4,5-trimethoxyphenyl)carbonylamino]-9-azabicyclo[3.3.1]non-9-yl}acetamide;
N-[(3-fluorophenyl)methyl]-2-{3-[(3,4,5-trimethoxyphenyl)carbonylamino]-9-azabicyclo[3.3.1]non-9-yl}acetamide;
N-(2,5-dichlorophenyl)-2-{3-[(3,4,5-trimethoxyphenyl)carbonylamino]-9-azabicyclo[3.3.1]non-9-yl}acetamide;
2-{3-[(5,6-dimethoxy(3-pyridyl))carbonylamino]-9-azabicyclo[3.3.1]non-9-yl}-N-(2-chlorophenyl)acetamide;
2-{3-[(5,6-dimethoxy(3-pyridyl))carbonylamino]-9-azabicyclo[3.3.1]non-9-yl}-N-phenylacetamide;
N-(2-chlorophenyl)-2-{9-hydroxy-3-[(3,4,5-trimethoxyphenyl)carbonylamino]-9-azabicyclo[3.3.1]non-9-yl}acetamide;
N-(2-chlorophenyl)-2-{3-[N-methyl(3,4,5-trimethoxyphenyl)carbonylamino]-9-azabicyclo[3.3.1]non-9-yl}acetamide;
N-(9-{[3-(4-chlorophenyl)(1,2,4-oxadiazol-5-yl)]methyl}-9-azabicyclo[3.3.1]non-3-yl)(3,4,5-trimethoxyphenyl)carboxamide;
[(3-methoxyphenyl)sulfonyl][9-benzyl-9-azabicyclo[3.3.1]non-3-yl]amine;
N-(2-chlorophenyl)-2-(3-{[(3-methoxyphenyl)sulfonyl]amino}-9-azabicyclo[3.3.1]non-9-yl)acetamide;
N-(2-chlorophenyl)-2-(3-{[(4-methoxyphenyl)sulfonyl]amino}-9-azabicyclo[3.3.1]non-9-yl)acetamide;
N-(9-{[N-(2-chlorophenyl)carbamoyl]methyl}-9-azabicyclo[3.3.1]non-3-yl)-2-(4-methoxyphenyl)acetamide;
N-(2-chlorophenyl)-2-({9-[(3,4,5-trimethoxyphenyl)carbonyl]-9-azabicyclo[3.3.1]non-3-yl}amino)acetamide;
N-(2-chlorophenyl)-N-methyl-2-{3-[N-methyl(3,4,5-trimethoxyphenyl)carbonylamino]-9-azabicyclo[3.3.1]non-9-yl}acetamide;
N-(2-chlorophenyl)-N-methyl-2-{3-[(3,4,5-trimethoxyphenyl)carbonylamino]-9-azabicyclo[3.3.1]non-9-yl}acetamide;
N-(2,3-dichlorophenyl)-2-{3-[(2-methoxyphenyl)carbonylamino]-9-azabicyclo[3.3.1]non-9-yl}acetamide;
N-(2,4-dichlorophenyl)-2-{3-[(2-methoxyphenyl)carbonylamino]-9-azabicyclo[3.3.1]non-9-yl}acetamide;
N-(2,3-dichlorophenyl)-2-(3-{[3-(dimethylamino)phenyl]carbonylamino}-9-azabicyclo[3.3.1]non-9-yl)acetamide;
N-(2,4-dichlorophenyl)-2-(3-{[3-(dimethylamino)phenyl]carbonylamino}-9-azabicyclo[3.3.1]non-9-yl)acetamide;
N-(2-chlorophenyl)-2-{3-[(2-methoxyphenyl)carbonylamino]-9-azabicyclo[3.3.1]non-9-yl}acetamide;
N-(2-chlorophenyl)-2-(3-f{[4-(trifluoromethoxy)phenyl]carbonylamino}-9-azabicyclo[3.3.1]non-9-yl)acetamide;
2-(3-{[4-(dimethylamino)phenyl]carbonylamino}-9-azabicyclo[3.3.1]non-9-yl)-N-(2-chlorophenyl)acetamide;
N-(2-chlorophenyl)-2-[3-(isoxazol-5-ylcarbonylamino)-9-azabicyclo[3.3.1]non-9-yl]acetamide;
N-(2-chlorophenyl)-2-{3-[(4-hydroxyphenyl)carbonylamino]-9-azabicyclo[3.3.1]non-9-yl}acetamide;
N-(2-chlorophenyl)-2-(3-{[4-(methylethoxy)phenyl]carbonylamino}-9-azabicyclo[3.3.1]non-9-yl)acetamide;
N-(2-chlorophenyl)-2-{3-[(4-prop-2-enyloxyphenyl)carbonylamino]-9-azabicyclo[3.3.1]non-9-yl}acetamide;
N-(2,3-dichlorophenyl)-2-{3-[(3-methoxyphenyl)carbonylamino]-9-azabicyclo[3.3.1]non-9-yl}acetamide;
N-(2,3-dichlorophenyl)-2-{3-[(4-methoxyphenyl)carbonylamino]-9-azabicyclo[3.3.1]non-9-yl}acetamide;
N-(2,3-dichlorophenyl)-2-[3-(cyclopentylcarbonylamino)-9-azabicyclo[3.3.1]non-9-yl]acetamide;
N-(2,3-dichlorophenyl)-2-{3-[(2-methyl(1,3-thiazol-4-yl))carbonylamino]-9-azabicyclo[3.3.1]non-9-yl}acetamide;
N-(2,3-dichlorophenyl)-2-{3-[(2-fluoro-3-methoxyphenyl)carbonylamino]-9-azabicyclo[3.3.1]non-9-yl}acetamide;
N-(2,3-dichlorophenyl)-2-{3-[(3-fluoro-4-methoxyphenyl)carbonylamino]-9-azabicyclo[3.3.1]non-9-yl}acetamide;
N-(2-chlorophenyl)-2-{3-[(3-methoxyphenyl)carbonylamino]-9-azabicyclo[3.3.1]non-9-yl}acetamide;
N-(2-chlorophenyl)-2-{3-[(4-methoxyphenyl)carbonylamino]-9-azabicyclo[3.3.1]non-9-yl}acetamide;
N-(2,4-dichlorophenyl)-2-{3-[(3-methoxyphenyl)carbonylamino]-9-azabicyclo[3.3.1]non-9-yl}acetamide;
2-[3-(2H-benzo[3,4-d]1,3-dioxolen-5-ylcarbonylamino)-9-azabicyclo[3.3.1]non-9-yl]-N-(2-chlorophenyl)acetamide;
N-(2,4-dichlorophenyl)-2-{3-[(4-methoxyphenyl)carbonylamino]-9-azabicyclo[3.3.1]non-9-yl}acetamide;
N-(2-chlorophenyl)-2-(3-{[(3,4,5-trimethoxyphenyl)methyl]amino}-9-azabicyclo[3.3.1]non-9-yl)acetamide;
N-{9-[2-(phenylcarbonylamino)acetyl]-9-azabicyclo[3.3.1]non-3-yl}(3,4,5-trimethoxyphenyl)carboxamide;
N-(2-chlorophenyl)-2-{3-[2-(3,4,5-trimethoxyphenyl)acetylamino]-9-azabicyclo[3.3.1]non-9-yl}acetamide;
2-[3-(benzimidazol-6-ylcarbonylamino)-9-azabicyclo[3.3.1]non-9-yl]-N-(2-chlorophenyl)acetamide;
N-[9-(benzothiazol-2-ylmethyl)-9-azabicyclo[3.3.1]non-3-yl](3,4,5-trimethoxyphenyl)carboxamide;
N-(2-chlorophenyl)-2-[3-(indol-5-ylcarbonylamino)-9-azabicyclo[3.3.1]non-9-yl]acetamide;
N-(2-chlorophenyl)-2-(3-{[2-(4-methoxyphenyl)-2-oxoethyl]amino}-9-azabicyclo[3.3.1]non-9-yl)acetamide;
N-(2-chlorophenyl)-2-{3-[(4-oxocyclohexyl)carbonylamino]-9-azabicyclo[3.3.1]non-9-yl}acetamide;
N-(2,4-dichlorophenyl)-2-[3-(1-oxoisoindolin-2-yl)-9-azabicyclo[3.3.1]non-9-yl]acetamide;
N-(2-chlorophenyl)-2-{3-[(6-hydroxy(3-pyridyl))carbonylamino]-9-azabicyclo[3.3.1]non-9-yl}acetamide;
2-{3-[(4-amino-3-methoxyphenyl)carbonylamino]-9-azabicyclo[3.3.1]non-9-yl}-N-(2-chlorophenyl)acetamide;
N-(2-chlorophenyl)-2-methyl-2-{3-[(3,4,5-trimethoxyphenyl)carbonylamino]-9-azabicyclo[3.3.1]non-9-yl}propanamide;
N-(2-chlorophenyl)-2-{3-[(4-chlorophenyl)carbonylamino]-9-azabicyclo[3.3.1]non-9-yl}acetamide;
N-(2-chlorophenyl)-2-{3-[(4-methylphenyl)carbonylamino]-9-azabicyclo[3.3.1]non-9-yl}acetamide;
N-(2-chlorophenyl)-2-{3-[(4-fluorophenyl)carbonylamino]-9-azabicyclo[3.3.1]non-9-yl}acetamide
N-(2-chlorophenyl)-2-[3-(indol-6-ylcarbonylamino)-9-azabicyclo[3.3.1]non-9-yl]acetamide;
N-(9-{2-[(2-chlorophenyl)amino]ethyl}-9-azabicyclo[3.3.1]non-3-yl)(3,4,5-trimethoxyphenyl)carboxamide;
N-{9-[2-(phenylamino)ethyl]-9-azabicyclo[3.3.1]non-3-yl}(3,4,5-trimethoxyphenyl)carboxamide;
N-(2-chlorophenyl)-2-{3-[(3,4,5-trimethoxyphenyl)carbonylamino]-9-azabicyclo[3.3.1]non-9-yl}propanamide;
N-(2-chlorophenyl)-2-{3-[(3-methylpyrazol-5-yl)carbonylamino]-9-azabicyclo[3.3.1]non-9-yl}acetamide;
N-(2,3-dichlorophenyl)-2-(3-{[4-(dimethylamino)phenyl]carbonylamino}-9-azabicyclo[3.3.1]non-9-yl)acetamide;
N-[9-(2-morpholin-4-yl-2-oxoethyl)-9-azabicyclo[3.3.1]non-3-yl](3,4,5-trimethoxyphenyl)carboxamide;
(2S,3S,4S,5R,6R)-6-[(6S,7S,1R,2R,10R,18R,19R,20R,21R)-18-((2Z)-2-methylbut-2-enoyloxy)-19-acetyloxy-6,20-bis(hydroxymethyl)-21-hydroxy-1,2,6,10,17,17-hexamethylpentacyclo[12.8.0.0<2,11>0.0<5,10>0.0<15,20>]docos-13-en-7-yloxy]-3,5-bis[(2S,4S,5S,3R,6R)-3,4,5-trihydroxy-6-(hydroxymethyl)(2H-3,4,5,6-tetrahydropyran-2-yl)oxy]-4-hydroxy-2H-3,4,5,6-tetrahydropyran-2-carboxylic acid;
2-{3-[(2,6-dimethoxyphenyl)carbonylamino]-9-azabicyclo[3.3.1]non-9-yl}-N-phenylacetamide;
2-{3-[(2,6-dimethoxyphenyl)carbonylamino]-9-azabicyclo[3.3.1]non-9-yl}-N-(2-chlorophenyl)acetamide;
2-{3-[(2,6-dimethoxyphenyl)carbonylamino]-9-azabicyclo[3.3.1]non-9-yl}-N-(4-chlorophenyl)acetamide;
N-(2,3-dichlorophenyl)-2-{3-[(2,6-dimethoxyphenyl)carbonylamino]-9-azabicyclo[3.3.1]non-9-yl}acetamide;
2-{3-[(2-methoxyphenyl)carbonylamino]-9-azabicyclo[3.3.1]non-9-yl}-N-phenylacetamide;
N-(2,5-dichlorophenyl)-2-{3-[(2-methoxyphenyl)carbonylamino]-9-azabicyclo[3.3.1]non-9-yl}acetamide;
2-{3-[(2-methoxyphenyl)carbonylamino]-9-azabicyclo[3.3.1]non-9-yl}-N-[2-(trifluoromethyl)phenyl]acetamide;
(9-{[N-(2-chlorophenyl)carbamoyl]methyl}-9-azabicyclo[3.3.1]non-3-yl)(2-methoxyphenyl);
(9-{[N-(2-chlorophenyl)carbamoyl]methyl}-9-azabicyclo[3.3.1]non-3-yl)(3-methoxyphenyl);
(9-{[N-(2-chlorophenyl)carbamoyl]methyl}-9-azabicyclo[3.3.1]non-3-yl)(4-methoxyphenyl);
1-[(9-{[N-(2-chlorophenyl)carbamoyl]methyl}-9-azabicyclo[3.3.1]non-3-yl)ureido]-4-methoxybutaneurea;
1-[(9-{[N-(2-chlorophenyl)carbamoyl]methyl}-9-azabicyclo[3.3.1]non-3-yl)ureido]-5-methoxypentaneurea;
N-(2-chlorophenyl)-2-[3-(morpholin-4-ylcarbonylamino)-9-azabicyclo[3.3.1]non-9-yl]acetamide;
N-(2-chlorophenyl)-2-{3-[(4-oxopiperidyl)carbonylamino]-9-azabicyclo[3.3.1]non-9-yl}acetamide;
N-(4-chlorophenyl)-2-methyl-2-{3-[(3,4,5-trimethoxyphenyl)carbonylamino]-9-azabicyclo[3.3.1]non-9-yl}propanamide;
N-(3-chlorophenyl)-2-methyl-2-{3-[(3,4,5-trimethoxyphenyl)carbonylamino]-9-azabicyclo[3.3.1]non-9-yl}propanamide;
N-(3,4-dichlorophenyl)-2-{3-[(2-methoxyphenyl)carbonylamino]-9-azabicyclo[3.3.1]non-9-yl}acetamide;
N-(3,5-dichlorophenyl)-2-{3-[(2-methoxyphenyl)carbonylamino]-9-azabicyclo[3.3.1]non-9-yl}acetamide;
N-(2,6-dichlorophenyl)-2-{3-[(2-methoxyphenyl)carbonylamino]-9-azabicyclo[3.3.1]non-9-yl}acetamide;
N-{9-[(2,4-dichlorophenyl)methyl]-9-azabicyclo[3.3.1]non-3-yl}(2-methoxyphenyl)carboxamide;
N-{9-[(2,6-dichlorophenyl)methyl]-9-azabicyclo[3.3.1]non-3-yl}(2-methoxyphenyl)carboxamide;
N-{9-[(3,4-dichlorophenyl)methyl]-9-azabicyclo[3.3.1]non-3-yl}(2-methoxyphenyl)carboxamide;
N-{9-[(2-chlorophenyl)methyl]-9-azabicyclo[3.3.1]non-3-yl}(2-methoxyphenyl)carboxamide;
N-{9-[(3-chlorophenyl)methyl]-9-azabicyclo[3.3.1]non-3-yl}(2-methoxyphenyl)carboxamide;
N-{9-[(4-chlorophenyl)methyl]-9-azabicyclo[3.3.1]non-3-yl}(2-methoxyphenyl)carboxamide;
N-(2-fluorophenyl)-2-{3-[(2-methoxyphenyl)carbonylamino]-9-azabicyclo[3.3.1]non-9-yl}acetamide;
N-(2-bromophenyl)-2-{3-[(2-methoxyphenyl)carbonylamino]-9-azabicyclo[3.3.1]non-9-yl}acetamide;
N-(3-chlorophenyl)-2-{3-[(2-methoxyphenyl)carbonylamino]-9-azabicyclo[3.3.1]non-9-yl}acetamide;
N-(4-chlorophenyl)-2-{3-[(2-methoxyphenyl)carbonylamino]-9-azabicyclo[3.3.1]non-9-yl}acetamide;
N-(4-chlorophenyl)-2-{3-[(2-chlorophenyl)carbonylamino]-9-azabicyclo[3.3.1]non-9-yl}acetamide;
N-(4-chlorophenyl)-2-{3-[(2-fluorophenyl)carbonylamino]-9-azabicyclo[3.3.1]non-9-yl}acetamide;
N-(4-chlorophenyl)-2-{3-[(2-methylphenyl)carbonylamino]-9-azabicyclo[3.3.1]non-9-yl}acetamide;
N-(2-chlorophenyl)-2-{3-[N-(3,4,5-trimethoxyphenyl)carbamoyl]-9-azabicyclo[3.3.1]non-9-yl}acetamide;
N-(2-chlorophenyl)-2-{3-[N-(3-methoxyphenyl)carbamoyl]-9-azabicyclo[3.3.1]non-9-yl}acetamide;
N-{9-[(4-chlorobenzothiazol-2-yl)methyl]-9-azabicyclo[3.3.1]non-3-yl}(3,4,5-trimethoxyphenyl)carboxamide;
N-{9-[(5-chlorobenzothiazol-2-yl)methyl]-9-azabicyclo[3.3.1]non-3-yl}(3,4,5-trimethoxyphenyl)carboxamide;
N-{9-[(6-chlorobenzothiazol-2-yl)methyl]-9-azabicyclo[3.3.1]non-3-yl}(3,4,5-trimethoxyphenyl)carboxamide;
N-{9-[(7-chlorobenzothiazol-2-yl)methyl]-9-azabicyclo[3.3.1]non-3-yl}(3,4,5-trimethoxyphenyl)carboxamide;
N-[9-(benzoxazol-2-ylmethyl)-9-azabicyclo[3.3.1]non-3-yl](3,4,5-trimethoxyphenyl)carboxamide;
N-[9-(benzimidazol-2-ylmethyl)-9-azabicyclo[3.3.1]non-3-yl](3,4,5-trimethoxyphenyl)carboxamide;
N-(2-chlorophenyl)-2-{3-[(2-methoxyphenyl)carbonylamino]-9-azabicyclo[3.3.1]non-9-yl}-2-methylpropanamide;
N-(2-chlorophenyl)-2-{3-[(3-methoxyphenyl)carbonylamino]-9-azabicyclo[3.3.1]non-9-yl}-2-methylpropanamide;
N-(2-chlorophenyl)-2-{3-[(4-methoxyphenyl)carbonylamino]-9-azabicyclo[3.3.1]non-9-yl}-2-methylpropanamide;
2-{3-[(2,6-dimethoxyphenyl)carbonylamino]-9-azabicyclo[3.3.1]non-9-yl}-N-(2-chlorophenyl)-2-methylpropanamide;
N-(9-{[N-(2-chlorophenyl)carbamoyl]cyclobutyl}-9-azabicyclo[3.3.1]non-3-yl)(3,4,5-trimethoxyphenyl)carboxamide;
N-(2-chlorophenyl)-2-[3-(2-phenylacetylamino)-9-azabicyclo[3.3.1]non-9-yl]acetamide;
N-(2-chlorophenyl)-2-{3-[2-(3-methoxyphenyl)acetylamino]-9-azabicyclo[3.3.1]non-9-yl}acetamide;
N-(2-chlorophenyl)-2-{3-[2-(2-methoxyphenyl)acetylamino]-9-azabicyclo[3.3.1]non-9-yl}acetamide;
N-(2-chlorophenyl)-2-{3-[2-(4-chlorophenyl)acetylamino]-9-azabicyclo[3.3.1]non-9-yl}acetamide;
N-(2-chlorophenyl)-2-{3-[2-(4-methylphenyl)acetylamino]-9-azabicyclo[3.3.1]non-9-yl}acetamide;
N-(2-chlorophenyl)-2-{3-[2-(3-chlorophenyl)acetylamino]-9-azabicyclo[3.3.1]non-9-yl}acetamide;
N-(2-chlorophenyl)-2-{3-[2-(3-methylphenyl)acetylamino]-9-azabicyclo[3.3.1]non-9-yl}acetamide;
N-(3-chlorophenyl)-2-{3-[2-(2-methoxyphenyl)acetylamino]-9-azabicyclo[3.3.1]non-9-yl}acetamide;
N-(3-chlorophenyl)-2-{3-[2-(3-methoxyphenyl)acetylamino]-9-azabicyclo[3.3.1]non-9-yl}acetamide;
N-(3-chlorophenyl)-2-{3-[2-(4-methoxyphenyl)acetylamino]-9-azabicyclo[3.3.1]non-9-yl}acetamide;
N-(4-chlorophenyl)-2-{3-[2-(2-methoxyphenyl)acetylamino]-9-azabicyclo[3.3.1]non-9-yl}acetamide;
N-(4-chlorophenyl)-2-{3-[2-(3-methoxyphenyl)acetylamino]-9-azabicyclo[3.3.1]non-9-yl}acetamide;
N-(4-chlorophenyl)-2-{3-[2-(4-methoxyphenyl)acetylamino]-9-azabicyclo[3.3.1]non-9-yl}acetamide;
N-(2-chlorophenyl)-2-{3-[(2-fluorophenyl)carbonylamino]-9-azabicyclo[3.3.1]non-9-yl}acetamide;
N-(2-chlorophenyl)-2-{3-[(2-chlorophenyl)carbonylamino]-9-azabicyclo[3.3.1]non-9-yl}acetamide;
N-(2-chlorophenyl)-2-{3-[(2-methylphenyl)carbonylamino]-9-azabicyclo[3.3.1]non-9-yl}acetamide;
N-{9-[(4-chlorobenzothiazol-2-yl)methyl]-9-azabicyclo[3.3.1]non-3-yl}benzamide;
N-{9-[(4-chlorobenzothiazol-2-yl)methyl]-9-azabicyclo[3.3.1]non-3-yl}(4-methoxyphenyl)carboxamide;
N-{9-[(4-chlorobenzothiazol-2-yl)methyl]-9-azabicyclo[3.3.1]non-3-yl}(3-chlorophenyl)carboxamide;
N-{9-[(4-chlorobenzothiazol-2-yl)methyl]-9-azabicyclo[3.3.1]non-3-yl}(4-chlorophenyl)carboxamide;
N-{9-[(4-bromobenzothiazol-2-yl)methyl]-9-azabicyclo[3.3.1]non-3-yl}(3,4,5-trimethoxyphenyl)carboxamide;
N-{9-[(4-fluorobenzothiazol-2-yl)methyl]-9-azabicyclo[3.3.1]non-3-yl}(3,4,5-trimethoxyphenyl)carboxamide;
N-{9-[(4-methylbenzothiazol-2-yl)methyl]-9-azabicyclo[3.3.1]non-3-yl}(3,4,5-trimethoxyphenyl)carboxamide;
N-(9-{[4-(trifluoromethyl)benzothiazol-2-yl]methyl}-9-azabicyclo[3.3.1]non-3-yl)(3,4,5-trimethoxyphenyl)carboxamide;
N-{9-[(4-methoxybenzothiazol-2-yl)methyl]-9-azabicyclo[3.3.1]non-3-yl}(3,4,5-trimethoxyphenyl)carboxamide;
N-{9-[(6-methoxybenzothiazol-2-yl)methyl]-9-azabicyclo[3.3.1]non-3-yl}(3,4,5-trimethoxyphenyl)carboxamide;
N-cyclopentyl-2-{3-[(3,4,5-trimethoxyphenyl)carbonylamino]-9-azabicyclo[3.3.1]non-9-yl}acetamide;
N-[9-(2-oxo-2-pyrrolidinylethyl)-9-azabicyclo[3.3.1]non-3-yl](3,4,5-trimethoxyphenyl)carboxamide;
N-(2-chlorophenyl)-2-phenyl-2-{3-[(3,4,5-trimethoxyphenyl)carbonylamino]-9-azabicyclo[3.3.1]non-9-yl}acetamide;
N-{9-[(4-chlorobenzothiazol-2-yl)methyl]-9-azabicyclo[3.3.1]non-3-yl}(2-methoxyphenyl)carboxamide;
N-{9-[(4-chlorobenzothiazol-2-yl)methyl]-9-azabicyclo[3.3.1]non-3-yl}(2-methylphenyl)carboxamide;
2-{3-[(2-bromophenyl)carbonylamino]-9-azabicyclo[3.3.1]non-9-yl}-N-(4-chlorophenyl)acetamide;
N-(4-chlorophenyl)-2-{3-[(3-chlorophenyl)carbonylamino]-9-azabicyclo[3.3.1]non-9-yl}acetamide;
N-(4-chlorophenyl)-2-{3-[2-(3-chlorophenyl)acetylamino]-9-azabicyclo[3.3.1]non-9-yl}acetamide;
N-[9-(benzo[b]thiophen-2-ylmethyl)-9-azabicyclo[3.3.1]non-3-yl](3,4,5-trimethoxyphenyl)carboxamide;
N-(4-chlorophenyl)-2-(3-f{[2-(trifluoromethyl)phenyl]carbonylamino}-9-azabicyclo[3.3.1]non-9-yl)acetamide;
N-[9-(2-benzothiazol-2-ylethyl)-9-azabicyclo[3.3.1]non-3-yl](3,4,5-trimethoxyphenyl)carboxamide;
N-[9-(2-quinolylmethyl)-9-azabicyclo[3.3.1]non-3-yl](3,4,5-trimethoxyphenyl)carboxamide;
N-[9-(benzothiazol-2-ylcarbonyl)-9-azabicyclo[3.3.1]non-3-yl](3,4,5-trimethoxyphenyl)carboxamide;
N-{9-[(4-chlorobenzothiazol-2-yl)methyl]-9-azabicyclo[3.3.1]non-3-yl}-2-(2-chlorophenyl)acetamide;
N-{9-[(4-chlorobenzothiazol-2-yl)methyl]-9-azabicyclo[3.3.1]non-3-yl}-2-(3-methoxyphenyl)acetamide
N-{9-[(4-chlorobenzothiazol-2-yl)methyl]-9-azabicyclo[3.3.1]non-3-yl}-2-(4-chlorophenyl)acetamide;
N-[9-(2-naphthylmethyl)-9-azabicyclo[3.3.1]non-3-yl](3,4,5-trimethoxyphenyl)carboxamide;
N-[9-(2-oxo-2-phenylethyl)-9-azabicyclo[3.3.1]non-3-yl](3,4,5-trimethoxyphenyl)carboxamide;
N-{9-[2-(3-chlorophenyl)-2-oxoethyl]-9-azabicyclo[3.3.1]non-3-yl}(3,4,5-trimethoxyphenyl)carboxamide;
N-{9-[2-(3,4-dichlorophenyl)-2-oxoethyl]-9-azabicyclo[3.3.1]non-3-yl}(3,4,5-trimethoxyphenyl)carboxamide;
N-{9-[(4-chlorobenzothiazol-2-yl)methyl]-9-azabicyclo[3.3.1]non-3-yl}(2-fluorophenyl)carboxamide;
N-{9-[(4-chlorobenzothiazol-2-yl)methyl]-9-azabicyclo[3.3.1]non-3-yl}(2-chlorophenyl)carboxamide;
N-{9-[(4-chlorobenzothiazol-2-yl)methyl]-9-azabicyclo[3.3.1]non-3-yl}(3-methoxyphenyl)carboxamide;
(3,5-dimethoxyphenyl)-N-{9-[(4-chlorobenzothiazol-2-yl)methyl]-9-azabicyclo[3.3.1]non-3-yl}carboxamide;
(3,4-dimethoxyphenyl)-N-{9-[(4-chlorobenzothiazol-2-yl)methyl]-9-azabicyclo[3.3.1]non-3-yl}carboxamide;
N-{9-[(4-chlorobenzothiazol-2-yl)methyl]-9-azabicyclo[3.3.1]non-3-yl}-2-(2-methoxyphenyl)acetamide;
N-{9-[(4-chlorobenzothiazol-2-yl)methyl]-9-azabicyclo[3.3.1]non-3-yl}-2-(3-chlorophenyl)acetamide;
N-(2-chlorophenyl)-2-methyl-2-{3-[(2-methylphenyl)carbonylamino]-9-azabicyclo[3.3.1]non-9-yl}propanamide;
N-(2-chlorophenyl)-2-{3-[(2-chlorophenyl)carbonylamino]-9-azabicyclo[3.3.1]non-9-yl}-2-methylpropanamide;
N-(2-chlorophenyl)-2-{3-[(2-fluorophenyl)carbonylamino]-9-azabicyclo[3.3.1]non-9-yl}-2-methylpropanamide;
N-[9-(indol-2-ylmethyl)-9-azabicyclo[3.3.1]non-3-yl](3,4,5-trimethoxyphenyl)carboxamide;
N-{9-[(5-chlorobenzothiazol-2-yl)methyl]-9-azabicyclo[3.3.1]non-3-yl}(2-methoxyphenyl)carboxamide;
N-{9-[(6-chlorobenzothiazol-2-yl)methyl]-9-azabicyclo[3.3.1]non-3-yl}(2-methoxyphenyl)carboxamide;
N-{9-[(7-chlorobenzothiazol-2-yl)methyl]-9-azabicyclo[3.3.1]non-3-yl}(2-methoxyphenyl)carboxamide;
N-{9-[(4-chlorobenzothiazol-2-yl)methyl]-9-azabicyclo[3.3.1]non-3-yl}(phenylmethoxy)carboxamide;
{9-[(4-chlorobenzothiazol-2-yl)methyl]-9-azabicyclo[3.3.1]non-3-yl}benzylamine;
N-[9-(indol-3-ylmethyl)-9-azabicyclo[3.3.1]non-3-yl](3,4,5-trimethoxyphenyl)carboxamide;
N-(9-{[4-(trifluoromethyl)benzothiazol-2-yl]methyl}-9-azabicyclo[3.3.1]non-3-yl)(2,3,4-trimethoxyphenyl)carboxamide;
(3,4,5-triethoxyphenyl)-N-(9-{[4-(trifluoromethyl)benzothiazol-2-yl]methyl}-9-azabicyclo[3.3.1]non-3-yl)carboxamide;
(3-bromo-4-methoxyphenyl)-N-(9-{[4-(trifluoromethyl)benzothiazol-2-yl]methyl}-9-azabicyclo[3.3.1]non-3-yl)carboxamide;
(3,5-dimethoxy-4-methylphenyl)-N-(9-{[4-(trifluoromethyl)benzothiazol-2-yl]methyl}-9-azabicyclo[3.3.1]non-3-yl)carboxamide;
(2-chloro-3,4-dimethoxyphenyl)-N-(9-{[4-(trifluoromethyl)benzothiazol-2-yl]methyl}-9-azabicyclo[3.3.1]non-3-yl)carboxamide;
(5-chloro-3,4-dimethoxyphenyl)-N-(9-{[4-(trifluoromethyl)benzothiazol-2-yl]methyl}-9-azabicyclo[3.3.1]non-3-yl)carboxamide;
(3-ethoxy-4-methoxyphenyl)-N-(9-{[4-(trifluoromethyl)benzothiazol-2-yl]methyl}-9-azabicyclo[3.3.1]non-3-yl)carboxamide;
(4-methoxy-3-propoxyphenyl)-N-(9-{[4-(trifluoromethyl)benzothiazol-2-yl]methyl}-9-azabicyclo[3.3.1]non-3-yl)carboxamide;
N-{9-[(5-bromo(1,3-thiazol-2-yl))methyl]-9-azabicyclo[3.3.1]non-3-yl}(3,4,5-trimethoxyphenyl)carboxamide;
N-(9-{[5-(methylethyl)(1,3-thiazol-2-yl)]methyl}-9-azabicyclo[3.3.1]non-3-yl)(3,4,5-trimethoxyphenyl)carboxamide;
N-(9-{[5-(tert-butyl)(1,3-thiazol-2-yl)]methyl}-9-azabicyclo[3.3.1]non-3-yl)(3,4,5-trimethoxyphenyl)carboxamide;
(4-ethoxy-3,5-dimethoxyphenyl)-N-(9-{[4-(trifluoromethyl)benzothiazol-2-yl]methyl}-9-azabicyclo[3.3.1]non-3-yl)carboxamide;
(3,5-dimethoxy-4-propoxyphenyl)-N-(9-{[4-(trifluoromethyl)benzothiazol-2-yl]methyl}-9-azabicyclo[3.3.1]non-3-yl)carboxamide;
[3,5-dimethoxy-4-(methylethoxy)phenyl]-N-(9-{[4-(trifluoromethyl)benzothiazol-2-yl]methyl}-9-azabicyclo[3.3.1]non-3-yl)carboxamide;
[3,5-dimethoxy-4-(phenylmethoxy)phenyl]-N-(9-{[4-(trifluoromethyl)benzothiazol-2-yl]methyl}-9-azabicyclo[3.3.1]non-3-yl)carboxamide;
(4-hydroxy-3,5-dimethoxyphenyl)-N-(9-{[4-(trifluoromethyl)benzothiazol-2-yl]methyl}-9-azabicyclo[3.3.1]non-3-yl)carboxamide;
(3,5-dimethoxy-4-prop-2-enyloxyphenyl)-N-(9-{[4-(trifluoromethyl)benzothiazol-2-yl]methyl}-9-azabicyclo[3.3.1]non-3-yl)carboxamide;
(4-methoxy-3,5-dimethylphenyl)-N-(9-{[4-(trifluoromethyl)benzothiazol-2-yl]methyl}-9-azabicyclo[3.3.1]non-3-yl)carboxamide;
(3,5-dichloro-4-methoxyphenyl)-N-(9-{[4-(trifluoromethyl)benzothiazol-2-yl]methyl}-9-azabicyclo[3.3.1]non-3-yl)carboxamide;
(3,5-dibromo-4-methoxyphenyl)-N-(9-{[4-(trifluoromethyl)benzothiazol-2-yl]methyl}-9-azabicyclo[3.3.1]non-3-yl)carboxamide;
(3,4-dimethoxyphenyl)-N-(9-{[4-(trifluoromethyl)benzothiazol-2-yl]methyl}-9-azabicyclo[3.3.1]non-3-yl)carboxamide;
(4-ethoxy-3-methoxyphenyl)-N-(9-{[4-(trifluoromethyl)benzothiazol-2-yl]methyl}-9-azabicyclo[3.3.1]non-3-yl)carboxamide;
(3-methoxy-4-propoxyphenyl)-N-(9-{[4-(trifluoromethyl)benzothiazol-2-yl]methyl}-9-azabicyclo[3.3.1]non-3-yl)carboxamide;
[3-methoxy-4-(methylethoxy)phenyl]-N-(9-{[4-(trifluoromethyl)benzothiazol-2-yl]methyl}-9-azabicyclo[3.3.1]non-3-yl)carboxamide;
(3-methoxy-4-prop-2-enyloxyphenyl)-N-(9-{[4-(trifluoromethyl)benzothiazol-2-yl]methyl}-9-azabicyclo[3.3.1]non-3-yl)carboxamide;
[3-methoxy-4-(phenylmethoxy)phenyl]-N-(9-{[4-(trifluoromethyl)benzothiazol-2-yl]methyl}-9-azabicyclo[3.3.1]non-3-yl)carboxamide;
(3-fluoro-4-methoxyphenyl)-N-(9-{[4-(trifluoromethyl)benzothiazol-2-yl]methyl}-9-azabicyclo[3.3.1]non-3-yl)carboxamide;
(3-chloro-4-methoxyphenyl)-N-(9-{[4-(trifluoromethyl)benzothiazol-2-yl]methyl}-9-azabicyclo[3.3.1]non-3-yl)carboxamide;
(4-methoxy-3-methylphenyl)-N-(9-{[4-(trifluoromethyl)benzothiazol-2-yl]methyl}-9-azabicyclo[3.3.1]non-3-yl)carboxamide;
(3-bromo-4-methoxyphenyl)-N-(9-{[4-(trifluoromethyl)benzothiazol-2-yl]methyl}-9-azabicyclo[3.3.1]non-3-yl)carboxamide;
[4-methoxy-3-(methylethoxy)phenyl]-N-(9-{[4-(trifluoromethyl)benzothiazol-2-yl]methyl}-9-azabicyclo[3.3.1]non-3-yl)carboxamide;
(4-methoxy-3-prop-2-enyloxyphenyl)-N-(9-{[4-(trifluoromethyl)benzothiazol-2-yl]methyl}-9-azabicyclo[3.3.1]non-3-yl)carboxamide;
[4-methoxy-3-(phenylmethoxy)phenyl]-N-(9-{[4-(trifluoromethyl)benzothiazol-2-yl]methyl}-9-azabicyclo[3.3.1]non-3-yl)carboxamide;
N-[9-(1,3-thiazol-2-ylmethyl)-9-azabicyclo[3.3.1]non-3-yl](3,4,5-trimethoxyphenyl)carboxamide;
N-{9-[(4-methyl(1,3-thiazol-2-yl))methyl]-9-azabicyclo[3.3.1]non-3-yl}(3,4,5-trimethoxyphenyl)carboxamide;
N-(9-{[4-(trifluoromethyl)(1,3-thiazol-2-yl)]methyl}-9-azabicyclo[3.3.1]non-3-yl)(3,4,5-trimethoxyphenyl)carboxamide;
N-{9-[(4-phenyl(1,3-thiazol-2-yl))methyl]-9-azabicyclo[3.3.1]non-3-yl}(3,4,5-trimethoxyphenyl)carboxamide;
ethyl 2-({3-[(3,4,5-trimethoxyphenyl)carbonylamino]-9-azabicyclo[3.3.1]non-9-yl}methyl)-1,3-thiazole-4-carboxylate;
9-{[4-(trifluoromethyl)benzothiazol-2-yl]methyl}-3,9-diazabicyclo[3.3.1]non-3-yl 3,4,5-trimethoxyphenyl ketone;
1-(9-{[4-(trifluoromethyl)benzothiazol-2-yl]methyl}-3,9-diazabicyclo[3.3.1]non-3-yl)-2-(3,4,5-trimethoxyphenyl)ethan-1-one;
(4-cyclopentyloxy-3-methoxyphenyl)-N-(9-{[4-(trifluoromethyl)benzothiazol-2-yl]methyl}-9-azabicyclo[3.3.1]non-3-yl)carboxamide;
[4-(cyclopropylmethoxy)-3-methoxyphenyl]-N-(9-{[4-(trifluoromethyl)benzothiazol-2-yl]methyl}-9-azabicyclo[3.3.1]non-3-yl)carboxamide;
[4-(difluoromethoxy)-3-methoxyphenyl]-N-(9-{[4-(trifluoromethyl)benzothiazol-2-yl]methyl}-9-azabicyclo[3.3.1]non-3-yl)carboxamide;
[3-methoxy-4-(2,2,2-trifluoroethoxy)phenyl]-N-(9-{[4-(trifluoromethyl)benzothiazol-2-yl]methyl}-9-azabicyclo[3.3.1]non-3-yl)carboxamide;
[4-(2,2-difluoroethoxy)-3-methoxyphenyl]-N-(9-{[4-(trifluoromethyl)benzothiazol-2-yl]methyl}-9-azabicyclo[3.3.1]non-3-yl)carboxamide;
[3-methoxy-4-(2-methylpropoxy)phenyl]-N-(9-{[4-(trifluoromethyl)benzothiazol-2-yl]methyl}-9-azabicyclo[3.3.1]non-3-yl)carboxamide;
[3-methoxy-4-(methylpropoxy)phenyl]-N-(9-{[4-(trifluoromethyl)benzothiazol-2-yl]methyl}-9-azabicyclo[3.3.1]non-3-yl)carboxamide;
[4-(ethylpropoxy)-3-methoxyphenyl]-N-(9-{[4-(trifluoromethyl)benzothiazol-2-yl]methyl}-9-azabicyclo[3.3.1]non-3-yl)carboxamide;
[3-methoxy-4-(3-methylbut-2-enyloxy)phenyl]-N-(9-{[4-(trifluoromethyl)benzothiazol-2-yl]methyl}-9-azabicyclo[3.3.1]non-3-yl)carboxamide;
[4-(ethoxymethoxy)-3-methoxyphenyl]-N-(9-{[4-(trifluoromethyl)benzothiazol-2-yl]methyl}-9-azabicyclo[3.3.1]non-3-yl)carboxamide;
(3-methoxy-4-oxetan-3-yloxyphenyl)-N-(9-{[4-(trifluoromethyl)benzothiazol-2-yl]methyl}-9-azabicyclo[3.3.1]non-3-yl)carboxamide;
(4-amino-3-methoxyphenyl)-N-(9-{[4-(trifluoromethyl)benzothiazol-2-yl]methyl}-9-azabicyclo[3.3.1]non-3-yl)carboxamide;
{3-methoxy-4-[(methylethyl)amino]phenyl}-N-(9-{[4-(trifluoromethyl)benzothiazol-2-yl]methyl}-9-azabicyclo[3.3.1]non-3-yl)carboxamide;
[3-amino-4-(methylethoxy)phenyl]-N-(9-{[4-(trifluoromethyl)benzothiazol-2-yl]methyl}-9-azabicyclo[3.3.1]non-3-yl)carboxamide;
N-{9-[(4,6-dichlorobenzothiazol-2-yl)methyl]-9-azabicyclo[3.3.1]non-3-yl}(3,4,5-trimethoxyphenyl)carboxamide;
N-{9-[(4,5-dichlorobenzothiazol-2-yl)methyl]-9-azabicyclo[3.3.1]non-3-yl}(3,4,5-trimethoxyphenyl)carboxamide;
N-{9-[(4,7-dichlorobenzothiazol-2-yl)methyl]-9-azabicyclo[3.3.1]non-3-yl}(3,4,5-trimethoxyphenyl)carboxamide;
N-(3-{2,3-dimethoxy-5-[N-(9-{[4-(trifluoromethyl)benzothiazol-2-yl]methyl}-9-azabicyclo[3.3.1]non-3-yl)carbamoyl]phenoxy}propyl)-3-(3-methyl(1,2-diazirin-3-yl))propanamide; and
[4-(methylethoxy)phenyl]-N-(9-{[4-(trifluoromethyl)benzothiazol-2-yl]methyl}-9-azabicyclo[3.3.1]non-3-yl)carboxamide, or a pharmaceutically acceptable salt, solvate, or
N-oxide thereof.

Any combination of the groups described above for the various variables is contemplated herein. Throughout the specification, groups and substituents thereof are chosen by one skilled in the field to provide stable moieties and compounds.

### Further Forms of Compounds

In one aspect, the compound of Formula (I), (Ia), (Ib), (Ic), (Id), (II), or (IIa) possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all exo-, endo-, cis-, trans-, syn-, anti-, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

“Pharmaceutically acceptable” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound of Formula (I), (Ia), (Ib), (Ic), (Id), (II), or (IIa) with acids. Pharmaceutically acceptable salts are also obtained by reacting a compound of Formula (I), (Ia), (Ib), (Ic), (Id), (II), or (IIa) with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

In one aspect, compounds are synthesized as described in the Examples section.

### Definitions

In the following description, certain specific details are set forth in order to provide a thorough understanding of various embodiments. However, one skilled in the art will understand that the current disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

The term “oxo” refers to the ═O substituent.

The term “thioxo” refers to the ═S substituent.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

The term “alkylamino” refers to a radical of the formula —NHR or —NRR where each R is, independently, an alkyl radical as defined above. Unless stated otherwise specifically in the specification, an alkylamino group may be optionally substituted as described below.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “aromatic” refers to a planar ring having a delocalized it-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

The terms “carbocyclic” or “carbocycle” refer to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

The term “aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group is partially reduced to form a cycloalkyl group defined herein. In some embodiments, an aryl group is fully reduced to form a cycloalkyl group defined herein.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “bridged” refers to any ring structure with two or more rings that contains a bridge connecting two bridgehead atoms. The bridgehead atoms are defined as atoms that are the part of the skeletal framework of the molecule and which are bonded to three or more other skeletal atoms. In some embodiments, the bridgehead atoms are C, N, or P. In some embodiments, the bridge is a single atom or a chain of atoms that connects two bridgehead atoms. In some embodiments, the bridge is a valence bond that connects two bridgehead atoms. In some embodiments, the bridged ring system is cycloalkyl. In some embodiments, the bridged ring system is heterocycloalkyl.

The term “fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with one or more N, S, and O atoms. The non-limiting examples of fused heterocyclyl or heteroaryl ring structures include 6-5 fused heterocycle, 6-6 fused heterocycle, 5-6 fused heterocycle, 5-5 fused heterocycle, 7-5 fused heterocycle, and 5-7 fused heterocycle.

The term “halo” or “halogen” refers to bromo, chloro, fluoro or iodo.

The term “haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

The term “haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

The term “fluoroalkyl” refers to an alkyl in which one or more hydrogen atoms are replaced by a fluorine atom. In one aspect, a fluoroalkyl is a C₁-C₆ fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₆ heteroalkyl. Representative heteroalkyl groups include, but are not limited to —OCH₂OMe, —OCH₂CH₂OH, —OCH₂CH₂OMe, or —OCH₂CH₂OCH₂CH₂NH₂.

The term “heteroalkylene” refers to an alkyl radical as described above where one or more carbon atoms of the alkyl is replaced with a O, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —OCH₂CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2N atoms. In some embodiments, heterocycloalkyls have from 2 to 10 carbons in the ring and 3 or 4N atoms. In some embodiments, heterocycloalkyls have from 2 to 12 carbons, 0-2N atoms, 0-2 O atoms, and 0-1 S atoms in the ring. In some embodiments, heterocycloalkyls have from 2 to 12 carbons, 1-3N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4N atoms in the ring. In some embodiments, a heteroaryl contains 1-4N atoms in the ring. In some embodiments, a heteroaryl contains 0-4N atoms, 0-1 O atoms, and 0-1 S atoms in the ring. In some embodiments, a heteroaryl contains 1-4N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂ alkyl, —C(═O)NH₂, —C(═O)NH (alkyl), —C(═O)N(alkyl)₂, —S(═O)₂NH₂, —S(═O)₂NH (alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₄ fluoroalkyl, C₁-C₄ heteroalkyl, C₁-C₄ alkoxy, C₁-C₄ fluoroalkoxy, —SC₁-C₄ alkyl, —S(═O)C₁-C₄ alkyl, and —S(═O)₂C₁-C₄ alkyl. In some embodiments, optional substituents are independently selected from D, halogen, —CN, —NH₂, —OH, —NH(CH₃), —N(CH₃)₂, —NH (cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

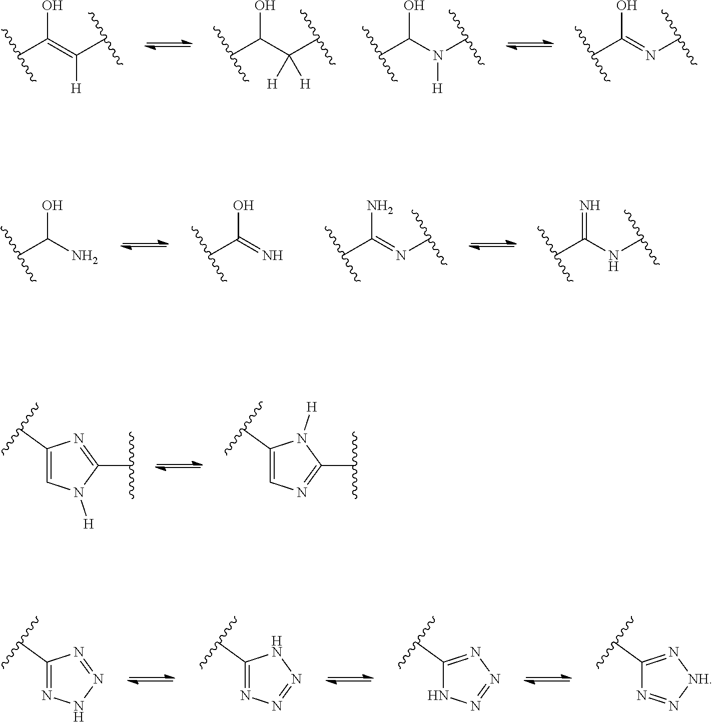

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I), (Ia), (Ib), (Ic), (Id), (II), or (IIa) and a co-agent, are both administered to a patient simultaneously in the form of a single entity or dosage. The term “non-fixed combination” means that the active ingredients, e.g. a compound of Formula (I), (Ia), (Ib), (Ic), (Id), (II), or (IIa) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “subject” or “patient” encompasses mammals. Examples of mammals include, but are not limited to, humans, bovines, rats, mice, dogs, monkeys, goats, sheep, cows, and deer. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “inflammation” refers to a localized response to various types of injury or infection, which is characterized by redness, heat, swelling, and pain, and often also including dysfunction or reduced mobility. Acute inflammation, according to Cruse and Lewis, ILLUSTRATED DICTIONARY OF IMMUNOLOGY, 2d ed. (CRC Press 2003), represents “an early defense mechanism to contain in infection and prevent its spread from the initial focus. When microbes multiply in host tissues, two principal defense mechanisms mounted against them are antibodies and leukocytes. The three major events in acute inflammation are (1) dilation of capillaries to increase blood flow; (2) changes in the microvasculature structure, leading to escape of plasma and proteins and leukocytes from the circulation; and (3) leukocyte emigration from the capillaries and accumulation at the site of injury.” Neutrophils escape from their endothelial location and are attracted by chemotaxis toward the site of injury. Prostaglandins and leukotrienes are formed, along with various cytokines. Non-specific defense mechanisms including natural killer (NK) cells are activated by cytokines.

### Administration and Pharmaceutical Composition

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Described herein are pharmaceutical compositions comprising an CXCR6 receptor inhibitor described herein. In some embodiments, the pharmaceutical composition refers to a mixture of an CXCR6 receptor inhibitor described herein with an excipient. In some embodiments, the excipient is a carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, antifoaming agent, antioxidant, preservative, or one or more combination thereof. The pharmaceutical composition facilitates administration of the CXCR6 receptor inhibitor described herein to an organism. In practicing the methods of treatment or use provided herein, therapeutically effective amounts of the CXCR6 receptor inhibitor described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The CXCR6 receptor inhibitor described herein can be used singly or in combination with one or more therapeutic agents as components of mixtures (as in combination therapy).

The pharmaceutical compositions described herein are administered to a subject by multiple administration routes, including but not limited to, oral, parenteral (e.g., intravenous, subcutaneous, intramuscular), intranasal, inhalation, buccal, topical, rectal, or transdermal administration routes. In some embodiments, pharmaceutical compositions described herein, which include an CXCR6 receptor inhibitor described herein, are formulated into any suitable dosage form, including but not limited to, emulsions suitable for injection, nanosuspensions suitable for injection, aqueous oral dispersions, liquids, gels, syrups, elixirs, slurries, suspensions, aerosols, controlled release formulations, fast melt formulations, effervescent formulations, lyophilized formulations, tablets, powders, pills, dragees, capsules, delayed release formulations, extended release formulations, pulsatile release formulations, multiparticulates formulations, and mixed immediate release and controlled release formulations.

Described herein are pharmaceutical compositions comprising an CXCR6 receptor inhibitor described herein formulated for oral administration. In some embodiments, the pharmaceutical composition for oral use is a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In some embodiments, the pharmaceutical composition for oral use is a solid dosage form, e.g., tablets, effervescent tablets, and capsules. In some embodiments, the solid dosage form are prepared by mixing particles of a CXCR6 receptor inhibitor described herein, with one or more pharmaceutical excipients to form a bulk blend composition. When referring to these bulk blend compositions as homogeneous, it is meant that the particles of the CXCR6 receptor inhibitor described herein, are dispersed evenly throughout the composition so that the composition may be subdivided into equally effective unit dosage forms, such as tablets, pills, and capsules. The individual unit dosages may also include film coatings, which disintegrate upon oral ingestion or upon contact with diluent.

The compositions disclosed herein can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort.

Pharmaceutical compositions may include compositions wherein the active ingredient (e.g. compounds described herein, including embodiments or examples) is contained in a therapeutically effective amount, i.e., in an amount effective to achieve its intended purpose. The actual amount effective for a particular application may depend, inter alia, on the condition being treated. When administered in methods to treat a disease, such compositions may contain an amount of active ingredient effective to achieve the desired result, e.g., modulating the activity of a target molecule, and/or reducing, eliminating, or slowing the progression of disease symptoms.

Generally, the CXCR6 receptor inhibitor described herein is administered in an amount effective for amelioration of the symptoms of the disease or disorder (i.e., a therapeutically effective amount). In some embodiments, the therapeutically effective amount is an amount that is capable of at least partially preventing or reversing a disease or disorder. In some embodiments, the dose required to obtain an effective amount varies depending on the agent, formulation, disease or disorder, and individual to whom the CXCR6 receptor inhibitor described herein is administered.

In some embodiments, the determination of the effective amount involves in vitro assays in which varying doses of the CXCR6 receptor inhibitor described herein are administered to cells in culture and the concentration of agent effective for ameliorating some or all symptoms is determined in order to calculate the concentration required in vivo. In some embodiments, the effective amounts are based on in vivo animal studies.

In some embodiments, the CXCR6 receptor inhibitor described herein is administered prior to, concurrently with and subsequent to the appearance of symptoms of a disease or disorder. In some embodiments, the CXCR6 receptor inhibitor described herein is administered to a subject with a family history of the disease or disorder, or who has a phenotype that may indicate a predisposition to a disease or disorder, or who has a genotype which predisposes the subject to the disease or disorder.

### Methods of Dosing and Treatment Regimens

In one embodiment, the compounds of Formula (I), (Ia), (Ib), (Ic), (Id), (II), or (IIa) are used in the preparation of medicaments for the treatment of diseases or conditions described herein. In addition, a method for treating any of the diseases or conditions described herein in a subject in need of such treatment, involves administration of pharmaceutical compositions that include at least one compound Formula (I), (Ia), (Ib), (Ic), (Id), (II), or (IIa) or a pharmaceutically acceptable salt, active metabolite, prodrug, or solvate thereof, in therapeutically effective amounts to said subject.

In certain embodiments, the compositions containing the compound of Formula (I), (Ia), (Ib), (Ic), (Id), (II), or (IIa) are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds of Formula (I), (Ia), (Ib), (Ic), (Id), (II), or (IIa) are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01 mg-5000 mg per day or from about 1 mg to about 1000 mg per day. In one embodiment, the desired dose is conveniently presented in a single dose or in divided doses.

### Combination Treatments

In certain instances, it is appropriate to administer at least one compound of Formula (I), (Ia), (Ib), (Ic), (Id), (II), or (IIa) in combination with another therapeutic agent.

In one specific embodiment, a compound of Formula (I), (Ia), (Ib), (Ic), (Id), (II), or (IIa) is co-administered with a second therapeutic agent, wherein the compound of Formula (I), (Ia), (Ib), (Ic), (Id), (II), or (IIa) and the second therapeutic agent modulate different aspects of the disease, disorder or condition being treated, thereby providing a greater overall benefit than administration of either therapeutic agent alone.

For combination therapies described herein, dosages of the co-administered compounds vary depending on the type of co-drug(s) employed, on the specific drug(s) employed, on the disease or condition being treated and so forth. In additional embodiments, when co-administered with one or more other therapeutic agents, the compound provided herein is administered either simultaneously with the one or more other therapeutic agents, or sequentially.

If administration is simultaneous, the multiple therapeutic agents are, by way of example only, provided in a single, unified form, or in multiple forms.

In some embodiments, a CXCR6 receptor inhibitor is used in combination with an anti-cancer therapy. In some embodiments, a CXCR6 receptor inhibitor is used in combination with conventional chemotherapy, radiotherapy, hormonal therapy, and/or immunotherapy. In some embodiments, a CXCR6 receptor inhibitor is used in combination with conventional chemotherapeutic agents including alkylating agents (e.g., temozolomide, cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, methotrexate, leucovorin, capecitabine, cytarabine, floxuridine, fludarabine, gemcitabine, pemetrexed, raltitrexed, etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin, etc.), EGFR inhibitors (e.g., gefitinib, erlotinib, etc.), and the like.

In some embodiments, a CXCR6 receptor inhibitor is used in combination with an anti-fibrotic drug. In some embodiments, the additional therapeutic agent used in the treatment of non-alcoholic fatty liver disease (NAFLD) or autoimmune hepatitis is vitamins C and E, betaine, ursodeoxycholic acid (UDCA), probucol, metformin, phenformin, buformin, losmian, pentoxifylline, troglitazone, rosiglitazone, proglitazone, tolbutamide, acetohexamide, tolazamide, chlorpropamide, glipizide, glibenclamide, glimepiride, gliclazide, glyclopyamide, gliquidone, repaglinide, nateglinide, pravastatin, atorvastatin, azathioprine, tluvastatin, lovastatin, pitavastatin, rosuvastatin, simvastatin, clofibrate, gemfibrozil, orlistat, fibrate, ezetimibe, prednisone, prednisolone, or any combination thereof. In some embodiments the additional therapeutic agent is an agent that is used in the treatment of autoimmune hepatitis, kidney, or lung injury. In some embodiments, the additional therapeutic agent used in the treatment of the autoimmune hepatitis, kidney, or lung injury is prednisone, prednisolone, deltasone, onintedanib, orapred odt, pirfenidone, veripred, rayos, pediapred, nintedanib, azathioprine, cyclophosphamide, mycophenolate mofetil, N-acetylcysteine, bosentan, sildenafil, interferon, endothelin-1, or any combination thereof. In some embodiments the kidney injury is acute kidney injury.

In some embodiments, a CXCR6 receptor inhibitor is used in combination with an anti-inflammatory drug. In some embodiments, the additional therapeutic agent used in the treatment of inflammation is aspirin, celecoxib, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, or any combination thereof.

### Methods of Treatment

In certain embodiments, also described herein is a method of treating a disease or condition mediated by CXCR6/CXCL16 signaling pathway in a mammal in need thereof comprising administering a therapeutically effective amount of a compound of Formula (I), (Ia), (Ib), (Ic), (Id), (II), or (IIa), or a pharmaceutically acceptable salt, solvate, or N-oxide thereof, to the mammal in need thereof.

In some embodiments, described herein is a method of treating cancer in a subject in need thereof comprising administering to the subject in need thereof a therapeutically effective amount of a compound of Formula (I), (Ia), (Ib), (Ic), (Id), (II), or (IIa).

In some embodiments, the cancer is a cancer described below. In some embodiments, the cancer is adrenal cortical cancer, anal cancer, aplastic anemia, bile duct cancer, bladder cancer, bone cancer, bone metastasis, Adult CNS brain tumors, Children CNS brain tumors, breast cancer, Castleman Disease, cervical cancer, Childhood Non-Hodgkin's lymphoma, colon and rectum (colorectal) cancer, endometrial cancer, esophagus cancer, Ewing's family of tumors, eye cancer, gallbladder cancer, gastrointestinal carcinoid tumors, gastrointestinal stromal tumors, gestational trophoblastic disease, glioblastoma multiforme, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine sarcoma, vaginal cancer, vulvar cancer, Waldenstrom's macroglobulinemia, cancers of viral origin and virus-associated cancers. In some instances, the cancer is selected from the group consisting of breast cancer, colon cancer, glioblastoma multiforme, lung cancer, melanoma, ovarian cancer, prostate cancer, and transformed stem cells cancer. In some instances, the cancer is breast cancer. In some instances, the cancer is triple-negative breast cancer. In some instances, the cancer is ovarian cancer.

### Breast Cancer

In one aspect, the pharmaceutical compositions disclosed herein provide a method of treating breast cancer. Several types of breast cancer exist that might be treated by the methods provided herein. A lobular carcinoma in situ and a ductal carcinoma in situ are breast cancers that have developed in the lobules and ducts, respectively, but have not spread to the fatty tissue surrounding the breast or to other areas of the body. An infiltrating (or invasive) lobular and a ductal carcinoma are cancers that have developed in the lobules and ducts, respectively, and have spread to either the breast's fatty tissue and/or other parts of the body. Other cancers of the breast that would benefit from treatment by the methods provided herein are medullary carcinomas, colloid carcinomas, tubular carcinomas, and inflammatory breast cancer. In some instances, the breast cancer is a triple-negative breast cancer.

Treatments available for breast cancer patients are surgery, immunotherapy, radiation therapy, chemotherapy, endocrine therapy, or a combination thereof. A lumpectomy and a mastectomy are two possible surgical procedures available for breast cancer patients.

Chemotherapy utilizes anti-tumor agents to prevent cancer cells from multiplying, invading, metastasizing and killing a patient. Several drugs are available to treat breast cancer, including cytotoxic drugs such as doxorubicin, cyclophosphamide, methotrexate, paclitaxel, thiotepa, mitoxantrone, vincristine, or combinations thereof. Endocrine therapy might be an effective treatment where the remaining breast tissue retains endocrine sensitivity. Agents administered for this therapy include tamoxifer, megestrol acetate, aminoglutethimide, fluoxymesterone, leuprolide, goserelin, and prednisone.

In some embodiments, described herein is a method of treating a breast cancer comprising administering to a subject in need thereof a therapeutically effective amount of a compound of Formula (I), (Ia), (Ib), (Ic), (Id), (II), or (IIa).

### Ovarian Cancer

In another aspect, provided herein is a method of treating ovarian cancer, including epithelial ovarian tumors. Preferably, provided herein is a method of treating an ovarian cancer selected from the following: an adenocarcinoma in the ovary and an adenocarcinoma that has migrated from the ovary into the abdominal cavity. Surgery, immunotherapy, chemotherapy, hormone therapy, radiation therapy, or a combination thereof are some possible treatments available for ovarian cancer. Some possible surgical procedures include debulking, and a unilateral or bilateral oophorectomy and/or a unilateral or bilateral salpingectomy.

Anti-cancer drugs that might be used include cyclophosphamide, etoposide, altretamine, and ifosfamide. Hormone therapy with the drug tamoxifen might be used to shrink ovarian tumors. Radiation therapy might be external beam radiation therapy and/or brachytherapy.

In some embodiments, described herein is a method of treating an ovarian cancer comprising administering to a subject in need thereof a therapeutically effective amount of a compound of Formula (I), (Ia), (Ib), (Ic), (Id), (II), or (IIa).

### Cervical Cancer

In another aspect, disclosed herein is a method of treating cervical cancer, preferably an adenocarcinoma in the cervix epithelial. Two main types of this cancer exist: squamous cell carcinoma and adenocarcinomas. The former constitutes about 80-90% of all cervical cancers and develops where the ectocervix (portion closest to the vagina) and the endocervix (portion closest to the uterus) join. The latter develop in the mucous-producing gland cells of the endocervix. Some cervical cancers have characteristics of both of these and are called adenosquamous carcinomas or mixed carcinomas.

The chief treatments available for cervical cancer are surgery, immunotherapy, radiation therapy and chemotherapy. Some possible surgical options are cryosurgery, a hysterectomy, and a radical hysterectomy. Radiation therapy for cervical cancer patients includes external beam radiation therapy or brachytherapy. Anti-cancer drugs that might be administered as part of chemotherapy to treat cervical cancer include cisplatin, carboplatin, hydroxyurea, irinotecan, bleomycin, vincristine, mitomycin, ifosfamide, fluorouracil, etoposide, methotrexate, and combinations thereof.

In some embodiments, described herein is a method of treating a cervical cancer comprising administering to a subject in need thereof a therapeutically effective amount of a compound of Formula (I), (Ia), (Ib), (Ic), (Id), (II), or (IIa).

### Prostate Cancer

In one other aspect, disclosed herein are methods to treat prostate cancer, preferably a prostate cancer selected from the following: an adenocarcinoma or an adenocarcinoma that has migrated to the bone. Prostate cancer develops in the prostate organ in men, which surrounds the first part of the urethra. The prostate has several cell types but 99% of tumors are adenocarcinomas that develop in the glandular cells responsible for generating seminal fluid.

Surgery, immunotherapy, radiation therapy, cryosurgery, hormone therapy, and chemotherapy are some treatments available for prostate cancer patients. Possible surgical procedures to treat prostate cancer include radical retropubic prostatectomy, a radical perineal prostatectomy, and a laparoscopic radical prostatectomy. Some radiation therapy options are external beam radiation, including three dimensional conformal radiation therapy, intensity modulated radiation therapy, and conformal proton beam radiation therapy. Brachytherapy (seed implantation or interstitial radiation therapy) is also an available method of treatment for prostate cancer. Cryosurgery is another possible method used to treat localized prostate cancer cells.

Hormone therapy, also called androgen deprivation therapy or androgen suppression therapy, might be used to treat prostate cancer. Several methods of this therapy are available including an orchiectomy in which the testicles, where 90% of androgens are produced, are removed. Another method is the administration of luteinizing hormone-releasing hormone (LHRH) analogs to lower androgen levels. The LHRH analogs available include leuprolide, goserelin, triptorelin, and histrelin. An LHRH antagonist might also be administered, such as abarelix.

Treatment with an anti-androgen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB).

Chemotherapy might be appropriate where a prostate tumor has spread outside the prostate gland and hormone treatment is not effective. Anti-cancer drugs such as doxorubicin, estramustine, etoposide, mitoxantrone, vinblastine, paclitaxel, docetaxel, carboplatin, and prednisone might be administered to slow the growth of prostate cancer, reduce symptoms and improve the quality of life.

In some embodiments, described herein is a method of treating a prostate cancer comprising administering to a subject in need thereof a therapeutically effective amount of a compound of Formula (I), (Ia), (Ib), (Ic), (Id), (II), or (IIa).

### Pancreatic Cancer

Some embodiments provide methods of treating pancreatic cancer, preferably a pancreatic cancer selected from the following: an epitheloid carcinoma in the pancreatic duct tissue and an adenocarcinoma in a pancreatic duct.

The most common type of pancreatic cancer is an adenocarcinoma, which occurs in the lining of the pancreatic duct. The possible treatments available for pancreatic cancer are surgery, immunotherapy, radiation therapy, and chemotherapy. Possible surgical treatment options include a distal or total pancreatectomy and a pancreaticoduodenectomy (Whipple procedure).

Radiation therapy might be an option for pancreatic cancer patients, specifically external beam radiation where radiation is focused on the tumor by a machine outside the body. Another option is intraoperative electron beam radiation administered during an operation.

Chemotherapy might be used to treat pancreatic cancer patients. Appropriate anti-cancer drugs include 5-fluorouracil (5-FU), mitomycin, ifosfamide, doxorubicin, streptozocin, chlorozotocin, and combinations thereof.

In some embodiments, described herein is a method of treating a pancreatic cancer comprising administering to a subject in need thereof a therapeutically effective amount of a compound of Formula (I), (Ia), (Ib), (Ic), (Id), (II), or (IIa).

### Bladder Cancer

Some embodiments provide methods of treating bladder cancer, preferably a transitional cell carcinoma in urinary bladder. Bladder cancers are urothelial carcinomas (transitional cell carcinomas) or tumors in the urothelial cells that line the bladder. The remaining cases of bladder cancer are squamous cell carcinomas, adenocarcinomas, and small cell cancers. Several subtypes of urothelial carcinomas exist depending on whether they are noninvasive or invasive and whether they are papillary, or flat. Noninvasive tumors are in the urothelium, the innermost layer of the bladder, while invasive tumors have spread from the urothelium to deeper layers of the bladder's main muscle wall. Invasive papillary urothelial carcinomas are slender finger-like projections that branch into the hollow center of the bladder and also grow outward into the bladder wall. Non-invasive papillary urothelial tumors grow towards the center of the bladder. While a non-invasive, flat urothelial tumor (also called a flat carcinoma in situ) is confined to the layer of cells closest to the inside hollow part of the bladder, an invasive flat urothelial carcinoma invades the deeper layer of the bladder, particularly the muscle layer.

To treat bladder cancer, surgery, radiation therapy, immunotherapy, chemotherapy, or a combination thereof might be applied. Some possible surgical options are a transurethral resection, a cystectomy, or a radical cystectomy. Radiation therapy for bladder cancer might include external beam radiation and brachytherapy.

Immunotherapy is another method that might be used to treat a bladder cancer patient. Typically this is accomplished intravesically, which is the administration of a treatment agent directly into the bladder by way of a catheter. One method is Bacillus Calmete-Guerin (BCG) where a bacterium sometimes used in tuberculosis vaccination is given directly to the bladder through a catheter. The body mounts an immune response to the bacterium, thereby attacking and killing the cancer cells.

Another method of immunotherapy is the administration of interferons, glycoproteins that modulate the immune response. Interferon alpha is often used to treat bladder cancer.

Anti-cancer drugs that nay be used in chemotherapy to treat bladder cancer include thitepa, methotrexate, vinblastine, doxorubicin, cyclophosphamide, paclitaxel, carboplatin, cisplatin, ifosfamide, gemcitabine, or combinations thereof.

In some embodiments, described herein is a method of treating a bladder cancer comprising administering to a subject in need thereof a therapeutically effective amount of a compound of Formula (I), (Ia), (Ib), (Ic), (Id), (II), or (IIa).

### Acute Myeloid Leukemia

Some embodiments provide methods of treating acute myeloid leukemia (AML), preferably acute promyelocytic leukemia (APL) in peripheral blood. AML begins in the bone marrow but can spread to other parts of the body including the lymph nodes, liver, spleen, central nervous system, and testes. It is acute meaning it develops quickly and might be fatal if not treated within a few months. AML is characterized by immature bone marrow cells usually granulocytes or monocytes, which continue to reproduce and accumulate.

AML might be treated by immunotherapy, radiation therapy, chemotherapy, bone marrow or peripheral blood stem cell transplantation, or a combination thereof. Radiation therapy includes external beam radiation and might have side effects. Anti-cancer drugs that might be used in chemotherapy to treat AML include cytarabine, anthracycline, anthracenedione, idarubicin, daunorubicin, idarubicin, mitoxantrone, thioguanine, vincristine, prednisone, etoposide, or a combination thereof.

Monoclonal antibody therapy might be used to treat AML patients. Small molecules or radioactive chemicals might be attached to these antibodies before administration to a patient in order to provide a means of killing leukemia cells in the body. The monoclonal antibody, gemtuzumab ozogamicin, which binds CD33 on AML cells, might be used to treat AML patients unable to tolerate prior chemotherapy regimens.

Bone marrow or peripheral blood stem cell transplantation might be used to treat AML patients. Some possible transplantation procedures are an allogenic or an autologous transplant.

In some embodiments, described herein is a method of treating a leukemia cancer comprising administering to a subject in need thereof a therapeutically effective amount of a compound of Formula (I), (Ia), (Ib), (Ic), (Id), (II), or (IIa).

In some embodiments, also described herein is a method of treating a leukemia subtype, e.g., Acute Lymphocytic Leukemia, Acute Myeloid Leukemia, Chronic Lymphocytic Leukemia, Chronic Myeloid Leukemia, Hairy Cell Leukemia, Myelodysplasia, and Myeloproliferative Disorders, comprising administering to a subject in need thereof a therapeutically effective amount of a compound of Formula (I), (Ia), (Ib), (Ic), (Id), (II), or (IIa).

### Lung Cancer

Some embodiments provide methods to treat lung cancer. The most common type of lung cancer is non-small cell lung cancer (NSCLC), which accounts for approximately 80-85% of lung cancers and is divided into squamous cell carcinomas, adenocarcinomas, and large cell undifferentiated carcinomas. Small cell lung cancer accounts for 15-20% of lung cancers.

Treatment options for lung cancer include surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy might be external beam radiation therapy or brachytherapy.

Some anti-cancer drugs that might be used in chemotherapy to treat lung cancer include cisplatin, carboplatin, paclitaxel, docetaxel, gemcitabine, vinorelbine, irinotecan, etoposide, vinblastine, gefitinib, ifosfamide, methotrexate, or a combination thereof. Photodynamic therapy (PDT) might be used to treat lung cancer patients.

In some embodiments, described herein is a method of treating a lung cancer comprising administering to a subject in need thereof a therapeutically effective amount of a compound of Formula (I), (Ia), (Ib), (Ic), (Id), (II), or (IIa).

### Skin Cancer

Some embodiments provide methods of treating skin cancer. There are several types of cancer that start in the skin. The most common types are basal cell carcinoma and squamous cell carcinoma, which are non-melanoma skin cancers. Actinic keratosis is a skin condition that sometimes develops into squamous cell carcinoma. Non-melanoma skin cancers rarely spread to other parts of the body. Melanoma, the rarest form of skin cancer, is more likely to invade nearby tissues and spread to other parts of the body. Different types of treatment are available for patients with non-melanoma and melanoma skin cancer and actinic keratosis including surgery, radiation therapy, chemotherapy and photodynamic therapy. Some possible surgical options for treatment of skin cancer are mohs micrographic surgery, simple excision, electrodessication and curettage, cryosurgery, laser surgery. Radiation therapy might be external beam radiation therapy or brachytherapy. Other types of treatments that are being tested in clinical trials are biologic therapy or immunotherapy, chemoimmunotherapy, topical chemotherapy with fluorouracil and photodynamic therapy.

In some embodiments, described herein is a method of treating a skin cancer comprising administering to a subject in need thereof a therapeutically effective amount of a compound of Formula (I), (Ia), (Ib), (Ic), (Id), (II), or (IIa).

### Eye Cancer, Retinoblastoma

Some embodiments provide methods to treat eye retinoblastoma. Retinoblastoma is a malignant tumor of the retina. Although retinoblastoma might occur at any age, it most often occurs in younger children, usually before the age of 5 years. The tumor might be in one eye only or in both eyes. Retinoblastoma is usually confined to the eye and does not spread to nearby tissue or other parts of the body. Treatment options that attempt to cure the patient and preserve vision include enucleation (surgery to remove the eye), radiation therapy, cryotherapy, photocoagulation, immunotherapy, thermotherapy and chemotherapy. Radiation therapy might be external beam radiation therapy or brachytherapy.

In some embodiments, described herein is a method of treating retinoblastoma comprising administering to a subject in need thereof a therapeutically effective amount of a compound of Formula (I), (Ia), (Ib), (Ic), (Id), (II), or (IIa).

### Eye Cancer, Intraocular Melanoma

Some embodiments provide methods to treat intraocular (eye) melanoma. Intraocular melanoma, a rare cancer, is a disease in which cancer cells are found in the part of the eye called the uvea. The uvea includes the iris, the ciliary body, and the choroid. Intraocular melanoma occurs most often in people who are middle aged. Treatments for intraocular melanoma include surgery, immunotherapy, radiation therapy and laser therapy. Surgery is the most common treatment of intraocular melanoma. Some possible surgical options are iridectomy, iridotrabeculectomy, iridocyclectomy, choroidectomy, enucleation and orbital exenteration. Radiation therapy might be external beam radiation therapy or brachytherapy. Laser therapy might be an intensely powerful beam of light to destroy the tumor, thermotherapy or photocoagulation.

In some embodiments, described herein is a method of treating intraocular melanoma comprising administering to a subject in need thereof a therapeutically effective amount of a compound of Formula (I), (Ia), (Ib), (Ic), (Id), (II), or (IIa).

### Endometrium Cancer

Some embodiments provide methods of treating endometrium cancer. Endometrial cancer is a cancer that starts in the endometrium, the inner lining of the uterus. Some of the examples of the cancer of uterus and endometrium include, but are not limited to, adenocarcinomas, adenoacanthomas, adenosquamous carcinomas, papillary serous adenocarcinomas, clear cell adenocarcinomas, uterine sarcomas, stromal sarcomas, malignant mixed mesodermal tumors, and leiomyosarcomas.

In some embodiments, described herein is a method of treating an endometrium cancer comprising administering to a subject in need thereof a therapeutically effective amount of a compound of Formula (I), (Ia), (Ib), (Ic), (Id), (II), or (IIa).

### Liver Cancer

Some embodiments provide methods to treat primary liver cancer (cancer that begins in the liver). Primary liver cancer can occur in both adults and children. Different types of treatments are available for patients with primary liver cancer. These include surgery, immunotherapy, radiation therapy, chemotherapy and percutaneous ethanol injection. The types of surgery that might be used are cryosurgery, partial hepatectomy, total hepatectomy and radiofrequency ablation. Radiation therapy might be external beam radiation therapy, brachytherapy, radiosensitizers or radiolabel antibodies. Other types of treatment include hyperthermia therapy and immunotherapy.

In some embodiments, described herein is a method of treating a liver cancer comprising administering to a subject in need thereof a therapeutically effective amount of a compound of Formula (I), (Ia), (Ib), (Ic), (Id), (II), or (IIa).

### Kidney Cancer

Some embodiments provide methods to treat kidney cancer. Kidney cancer (also called renal cell cancer or renal adenocarcinoma) is a disease in which malignant cells are found in the lining of tubules in the kidney. Kidney cancer might be treated by surgery, radiation therapy, chemotherapy and immunotherapy. Some possible surgical options to treat kidney cancer are partial nephrectomy, simple nephrectomy and radical nephrectomy. Radiation therapy might be external beam radiation therapy or brachytherapy. Stem cell transplant might be used to treat kidney cancer.

In some embodiments, described herein is a method of treating a kidney cancer comprising administering to a subject in need thereof a therapeutically effective amount of a compound of Formula (I), (Ia), (Ib), (Ic), (Id), (II), or (IIa).

### Thyroid Cancer

Some embodiments provide methods of treating thyroid cancer. Thyroid cancer is a disease in which cancer (malignant) cells are found in the tissues of the thyroid gland. The four main types of thyroid cancer are papillary, follicular, medullary and anaplastic. Thyroid cancer might be treated by surgery, immunotherapy, radiation therapy, hormone therapy and chemotherapy. Surgery is the most common treatment of thyroid cancer. Some possible surgical options for treatment of thyroid cancer are lobectomy, near-total thyroidectomy, total thyroidectomy and lymph node dissection. Radiation therapy might be external radiation therapy or might require intake of a liquid that contains radioactive iodine. Hormone therapy uses hormones to stop cancer cells from growing. In treating thyroid cancer, hormones might be used to stop the body from making other hormones that might make cancer cells grow.

In some embodiments, described herein is a method of treating a thyroid cancer comprising administering to a subject in need thereof a therapeutically effective amount of a compound of Formula (I), (Ia), (Ib), (Ic), (Id), (II), or (IIa). In some embodiments, the thyroid cancer is papillary thyroid carcinoma.

### AIDS Related Cancer

### AIDS-Related Lymphoma

Some embodiments provide methods of treating AIDS-related lymphoma. AIDS-related lymphoma is a disease in which malignant cells form in the lymph system of patients who have acquired immunodeficiency syndrome (AIDS). AIDS is caused by the human immunodeficiency virus (HIV), which attacks and weakens the body's immune system. The immune system is then unable to fight infection and diseases that invade the body. People with HIV disease have an increased risk of developing infections, lymphoma, and other types of cancer. Lymphomas are cancers that affect the white blood cells of the lymph system. Lymphomas are divided into two general types: Hodgkin's lymphoma and non-Hodgkin's lymphoma. Both Hodgkin's lymphoma and non-Hodgkin's lymphoma might occur in AIDS patients, but non-Hodgkin's lymphoma is more common. When a person with AIDS has non-Hodgkin's lymphoma, it is called an AIDS-related lymphoma. Non-Hodgkin's lymphomas might be indolent (slow-growing) or aggressive (fast-growing). AIDS-related lymphoma is usually aggressive. The three main types of AIDS-related lymphoma are diffuse large B-cell lymphoma, B-cell immunoblastic lymphoma and small non-cleaved cell lymphoma.

Treatment of AIDS-related lymphoma combines treatment of the lymphoma with treatment for AIDS. Patients with AIDS have weakened immune systems and treatment can cause further damage. For this reason, patients who have AIDS-related lymphoma are usually treated with lower doses of drugs than lymphoma patients who do not have AIDS. Highly-active antiretroviral therapy (HAART) is used to slow progression of HIV. Medicine to prevent and treat infections, which can be serious, is also used. AIDS-related lymphomas might be treated by chemotherapy, immunotherapy, radiation therapy, and high-dose chemotherapy with stem cell transplant. Radiation therapy might be external beam radiation therapy or brachytherapy. AIDS-related lymphomas can be treated by monoclonal antibody therapy.

In some embodiments, described herein is a method of treating an AIDS-related lymphoma comprising administering to a subject in need thereof a therapeutically effective amount of a compound of Formula (I), (Ia), (Ib), (Ic), (Id), (II), or (IIa).

### Kaposi's Sarcoma

Some embodiments provide methods of treating Kaposi's sarcoma. Kaposi's sarcoma is a disease in which cancer cells are found in the tissues under the skin or mucous membranes that line the mouth, nose, and anus. Classic Kaposi's sarcoma usually occurs in older men of Jewish, Italian, or Mediterranean heritage. This type of Kaposi's sarcoma progresses slowly, sometimes over 10 to 15 years. Kaposi's sarcoma might occur in people who are taking immunosuppressants. Kaposi's sarcoma in patients who have Acquired Immunodeficiency Syndrome (AIDS) is called epidemic Kaposi's sarcoma. Kaposi's sarcoma in people with AIDS usually spreads more quickly than other kinds of Kaposi's sarcoma and often is found in many parts of the body. Kaposi's sarcoma might be treated with surgery, chemotherapy, radiation therapy and immunotherapy. External radiation therapy is a common treatment of Kaposi's sarcoma. Some possible surgical options to treat Kaposi's Sarcoma are local excision, electrodessication and curettage, and cryotherapy.

In some embodiments, described herein is a method of treating Kaposi's sarcoma comprising administering to a subject in need thereof a therapeutically effective amount of a compound of Formula (I), (Ia), (Ib), (Ic), (Id), (II), or (IIa).

### Viral-Induced Cancers

Some embodiments provide methods of treating viral-induced cancers. Several common viruses are clearly or probable causal factors in the etiology of specific malignancies. These viruses either normally establish latency or few can become persistent infections. Oncogenesis is probably linked to an enhanced level of viral activation in the infected host, reflecting heavy viral dose or compromised immune control. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer. In general, these malignancies occur relatively early in life, typically peaking in middle-age or earlier.

### Virus-Induced Hepatocellular Carcinoma

The causal relationship between both HBV aid HCV and hepatocellular carcinoma or liver cancer is established through substantial epidemiologic evidence. Both appear to act via chronic replication in the liver by causing cell death and subsequent regeneration. Different types of treatments are available for patients with liver cancer. These include surgery, immunotherapy, radiation therapy, chemotherapy and percutaneous ethanol injection. The types of surgery that might be used are cryosurgery, partial hepatectomy, total hepatectomy and radiofrequency ablation. Radiation therapy might be external beam radiation therapy, brachytherapy, radiosensitizers or radiolabel antibodies. Other types of treatment include hyperthermia therapy and immunotherapy.

In some embodiments, described herein is a method of treating a virus-induced hepatocellular carcinoma comprising administering to a subject in need thereof a therapeutically effective amount of a compound of Formula (I), (Ia), (Ib), (Ic), (Id), (II), or (IIa).

### Viral-Induced Adult T Cell Leukemia/Lymphoma

The association between HTLV-1 and Adult T cell leukemia (ATL) is firmly established. Unlike the other oncogenic viruses found throughout the world, HTLV-1 is highly geographically restricted, being found primarily in southern Japan, the Caribbean, west and central Africa, and the South Pacific islands. Evidence for causality includes the monoclonal integration of viral genome in almost all cases of ATL in carriers. The risk factors for HTLV-1-associated malignancy appear to be perinatal infection, high viral load, and being male sex.

Adult T cell leukemia is a cancer of the blood and bone marrow. The standard treatments for adult T cell leukemia/lymphoma are radiation therapy, immunotherapy, and chemotherapy. Radiation therapy might be external beam radiation therapy or brachytherapy. Other methods of treating adult T cell leukemia/lymphoma include immunotherapy and high-dose chemotherapy with stem cell transplantation.

In some embodiments, described herein is a method of treating a viral-induced adult T cell leukemia/lymphoma comprising administering to a subject in need thereof a therapeutically effective amount of a compound of Formula (I), (Ia), (Ib), (Ic), (Id), (II), or (IIa).

### Viral-Induced Cervical Cancer

Infection of the cervix with human papillomavirus (HPV) is the most common cause of cervical cancer. Not all women with HPV infection, however, will develop cervical cancer. Cervical cancer usually develops slowly over time. Before cancer appears in the cervix, the cells of the cervix go through changes known as dysplasia, in which cells that are not normal begin to appear in the cervical tissue. Later, cancer cells start to grow and spread more deeply into the cervix and to surrounding areas. The standard treatments for cervical cancers are surgery, immunotherapy, radiation therapy and chemotherapy. The types of surgery that might be used are conization, total hysterectomy, bilateral salpingo-oophorectomy, radical hysterectomy, pelvic exenteration, cryosurgery, laser surgery, and loop electrosurgical excision procedure. Radiation therapy might be external beam radiation therapy or brachytherapy.

In some embodiments, described herein is a method of treating a viral-induced cervical cancer comprising administering to a subject in need thereof a therapeutically effective amount of a compound of Formula (I), (Ia), (Ib), (Ic), (Id), (II), or (IIa).

### CNS Cancers

Brain and spinal cord tumors are abnormal growths of tissue found inside the skull or the bony spinal column, which are the primary components of the central nervous system (CNS). Benign tumors are non-cancerous, and malignant tumors are cancerous. The CNS is housed within rigid, bony quarters (i.e., the skull and spinal column), so any abnormal growth, whether benign or malignant, can place pressure on sensitive tissues and impair function. Tumors that originate in the brain or spinal cord are called primary tumors. Most primary tumors are caused by out-of-control growth among cells that surround and support neurons. In a small number of individuals, primary tumors might result from specific genetic disease (e.g., neurofibromatosis, tuberous sclerosis) or from exposure to radiation or cancer-causing chemicals. The cause of most primary tumors remains a mystery.

The first test to diagnose brain and spinal column tumors is a neurological examination. Special imaging techniques (computed tomography, and magnetic resonance imaging, positron emission tomography) are also employed. Laboratory tests include the EEG and the spinal tap. A biopsy, a surgical procedure in which a sample of tissue is taken from a suspected tumor, helps doctors diagnose the type of tumor.

Tumors are classified according to the kind of cell from which the tumor seems to originate. The most common primary brain tumor in adults comes from cells in the brain called astrocytes that make up the blood-brain barrier and contribute to the nutrition of the central nervous system. These tumors are called gliomas (astrocytoma, anaplastic astrocytoma, or glioblastoma multiforme) and account for 65% of all primary central nervous system tumors. Some of the tumors are, but not limited to, Oligodendroglioma, Ependymoma, Meningioma, Lymphoma, Schwannoma, and Medulloblastoma.

### Neuroepithelial Tumors of the CNS

Astrocytic tumors, such as astrocytoma; anaplastic (malignant) astrocytoma, such as hemispheric, diencephalic, optic, brain stem, cerebellar; glioblastoma multiforme; pilocytic astrocytoma, such as hemispheric, diencephalic, optic, brain stem, cerebellar; subependymal giant cell astrocytoma; and pleomorphic xanthoastrocytoma. Oligodendroglial tumors, such as oligodendroglioma; and anaplastic (malignant) oligodendroglioma. Ependymal cell tumors, such as ependymoma; anaplastic ependymoma; myxopapillary ependymoma; and subependymoma. Mixed gliomas, such as mixed oligoastrocytoma; anaplastic (malignant) oligoastrocytoma; and others (e.g. ependymo-astrocytomas). Neuroepithelial tumors of uncertain origin, such as polar spongioblastoma; astroblastoma; and gliomatosis cerebri. Tumors of the choroid plexus, such as choroid plexus papilloma; and choroid plexus carcinoma (anaplastic choroid plexus papilloma). Neuronal and mixed neuronal-glial tumors, such as gangliocytoma; dysplastic gangliocytoma of cerebellum (Lhermitte-Duclos); ganglioglioma; anaplastic (malignant) ganglioglioma; desmoplastic infantile ganglioglioma, such as desmoplastic infantile astrocytoma; central neurocytoma; dysembryoplastic neuroepithelial tumor; olfactory neuroblastoma (esthesioneuroblastoma. Pineal Parenchyma Tumors, such as pineocytoma; pineoblastoma; and mixed pineocytoma/pineoblastoma. Tumors with neuroblastic or glioblastic elements (embryonal tumors), such as medulloepithelioma; primitive neuroectodermal tumors with multipotent differentiation, such as medulloblastoma; cerebral primitive neuroectodermal tumor; neuroblastoma; retinoblastoma; and ependymoblastoma.

### Other CNS Neoplasms

Tumors of the Sellar Region, such as pituitary adenoma; pituitary carcinoma; and craniopharyngioma. Hematopoietic tumors, such as primary malignant lymphomas; plasmacytoma; and granulocytic sarcoma. Germ Cell Tumors, such as germinoma; embryonal carcinoma; yolk sac tumor (endodermal sinus tumor); choriocarcinoma; teratoma; and mixed germ cell tumors. Tumors of the Meninges, such as meningioma; atypical meningioma; and anaplastic (malignant) meningioma. Non-menigothelial tumors of the meninges, such as Benign Mesenchymal; Malignant Mesenchymal; Primary Melanocytic Lesions; Hemopoietic Neoplasms; and Tumors of Uncertain Histogenesis, such as hemangioblastoma (capillary hemangioblastoma). Tumors of Cranial and Spinal Nerves, such as schwannoma (neurinoma, neurilemmoma); neurofibroma; malignant peripheral nerve sheath tumor (malignant schwannoma), such as epithelioid, divergent mesenchymal or epithelial differentiation, and melanotic. Local Extensions from Regional Tumors; such as paraganglioma (chemodectoma); chordoma; chodroma; chondrosarcoma; and carcinoma. Metastatic tumors, Unclassified Tumors and Cysts and Tumor-like Lesions, such as Rathke cleft cyst; Epidermoid; dermoid; colloid cyst of the third ventricle; enterogenous cyst; neuroglial cyst; granular cell tumor (choristoma, pituicytoma); hypothalamic neuronal hamartoma; nasal glial herterotopia; and plasma cell granuloma.

Chemotherapeutics available are, but not limited to, alkylating agents such as, Cyclophosphamide, Ifosphamide, Melphalan, Chlorambucil, BCNU, CCNU, Decarbazine, Procarbazine, Busulfan, and Thiotepa; antimetabolites such as, Methotrexate, 5-Fluorouracil, Cytarabine, Gemcitabine (Gemzar®), 6-mercaptopurine, 6-thioguanine, Fludarabine, and Cladribine; anthracyclins such as, daunorubicin. Doxorubicin, Idarubicin, Epirubicin and Mitoxantrone; antibiotics such as, Bleomycin; camptothecins such as, irinotecan and topotecan; taxanes such as, paclitaxel and docetaxel; and platinums such as, Cisplatin, carboplatin, and Oxaliplatin.

The treatments are surgery, radiation therapy, immunotherapy, hyperthermia, gene therapy, chemotherapy, and combination of radiation and chemotherapy. Doctors also might prescribe steroids to reduce the swelling inside the CNS.

In some embodiments, described herein is a method of treating a CNS cancer comprising administering to a subject in need thereof a therapeutically effective amount of a compound of Formula (I), (Ia), (Ib), (Ic), (Id), (II), or (IIa).

### PANS Cancers

The peripheral nervous system consists of the nerves that branch out from the brain and spinal cord. These nerves form the communication network between the CNS and the body parts. The peripheral nervous system is further subdivided into the somatic nervous system and the autonomic nervous system. The somatic nervous system consists of nerves that go to the skin and muscles and is involved in conscious activities. The autonomic nervous system consists of nerves that connect the CNS to the visceral organs such as the heart, stomach, and intestines. It mediates unconscious activities.

Acoustic neuromas are benign fibrous growths that arise from the balance nerve, also called the eighth cranial nerve or vestibulocochlear nerve. These tumors are non-malignant, meaning that they do not spread or metastasize to other parts of the body. The location of these tumors is deep inside the skull, adjacent to vital brain centers in the brain stem. As the tumors enlarge, they involve surrounding structures which have to do with vital functions. In the majority of cases, these tumors grow slowly over a period of years.

The malignant peripheral nerve sheath tumor (MPNST) is the malignant counterpart to benign soft tissue tumors such as neurofibromas and schwannomas. It is most common in the deep soft tissue, usually in close proximity of a nerve trunk. The most common sites include the sciatic nerve, brachial plexus, and sarcal plexus. The most common symptom is pain which usually prompts a biopsy. It is a rare, aggressive, and lethal orbital neoplasm that usually arises from sensory branches of the trigeminal nerve in adults. Malignant PNS tumor spreads along nerves to involve the brain, and most patients die within 5 years of clinical diagnosis. The MPNST might be classified into three major categories with epithelioid, mesenchymal or glandular characteristics. Some of the MPNST include but not limited to, Subcutaneous malignant epithelioid schwannoma with cartilaginous differentiation, Glandular malignant schwannoma, Malignant peripheral nerve sheath tumor with perineural differentiation, Cutaneous epithelioid malignant nerve sheath tumor with rhabdoid features, Superficial epithelioid MPNST, Triton Tumor (MPNST with rhabdomyoblastic differentiation), Schwannoma with rhabdomyoblastic differentiation. Rare MPNST cases contain multiple sarcomatous tissue types, especially osteosarcoma, chondrosarcoma and angiosarcoma. These have sometimes been indistinguishable from the malignant mesenchymoma of soft tissue.

Other types of PNS cancers include but not limited to, malignant fibrous cytoma, malignant fibrous histiocytoma, malignant meningioma, malignant mesothelioma, and malignant mixed Müllerian tumor.

The treatments are surgery, radiation therapy, immunotherapy, chemotherapy, and combination of radiation and chemotherapy.

In some embodiments, described herein is a method of treating a PNS cancer comprising administering to a subject in need thereof a therapeutically effective amount of a compound of Formula (I), (Ia), (Ib), (Ic), (Id), (II), or (IIa).

### Oral Cavity and Oropharyngeal Cancer

Management of patients with central nervous system (CNS) cancers remains a formidable task. Cancers such as hypopharyngeal cancer, laryngeal cancer, nasopharyngeal cancer, oropharyngeal cancer, and the like, have been treated with surgery, immunotherapy, chemotherapy, combination of chemotherapy, and radiation therapy. Etoposide and actinomycin D, two commonly used oncology agents that inhibit topoisomerase II, fail to cross the blood-brain barrier in useful amounts.

In some embodiments, described herein is a method of treating an oral cavity and oropharyngeal cancer comprising administering to a subject in need thereof a therapeutically effective amount of a compound of Formula (I), (Ia), (Ib), (Ic), (Id), (II), or (IIa).

### Stomach Cancer

Stomach cancer is the result of cell changes in the lining of the stomach. There are three main types of stomach cancers: lymphomas, gastric stromal tumors, and carcinoid tumors. In some embodiments, the stomach cancer is gastric adenocarcinoma. Lymphomas are cancers of the immune system tissue that are sometimes found in the wall of the stomach. Gastric stromal tumors develop from the tissue of the stomach wall. Carcinoid tumors are tumors of hormone-producing cells of the stomach.

The causes of stomach cancer continue to be debated. A combination of heredity and environment (diet, smoking, etc) are all thought to play a part. Common approaches to the treatment include surgery, immunotherapy, chemotherapy, radiation therapy, combination of chemotherapy, and radiation therapy or biological therapy.

In some embodiments, described herein is a method of treating a stomach cancer comprising administering to a subject in need thereof a therapeutically effective amount of a compound of Formula (I), (Ia), (Ib), (Ic), (Id), (II), or (IIa).

### Testicular Cancer

Testicular cancer is cancer that typically develops in one or both testicles in young men. Cancers of the testicle develop in certain cells known as germ cells. The 2 main types of germ cell tumors (GCTs) that occur in men are seminomas (60%) and nonseminomas (40%). Tumors can also arise in the supportive and hormone-producing tissues, or stroma, of the testicles. Such tumors are known as gonadal stromal tumors. The 2 main types are Leydig cell tumors and Sertoli cell tumors. Secondary testicular tumors are those that start in another organ and then spread to the testicle. Lymphoma is the most common secondary testicular cancer.

Common approaches to the treatment include surgery, immunotherapy, chemotherapy, radiation therapy, combination of chemotherapy, and radiation therapy or biological therapy. Several drugs are typically used to treat testicular cancer: Platinol (cisplatin), Vepesid or VP-16 (etoposide) and Blenoxane (bleomycin sulfate). Additionally, Ifex (ifosfamide), Velban (vinblastine sulfate) and others might be used.

In some embodiments, described herein is a method of treating a testicular cancer comprising administering to a subject in need thereof a therapeutically effective amount of a compound of Formula (I), (Ia), (Ib), (Ic), (Id), (II), or (IIa).

### Thymus Cancer

The thymus is a small organ located in the upper/front portion of your chest, extending from the base of the throat to the front of the heart. The thymus contains 2 main types of cells, thymic epithelial cells and lymphocytes. Thymic epithelial cells can give origin to thymomas and thymic carcinomas. Lymphocytes, whether in the thymus or in the lymph nodes, can become malignant and develop into cancers called Hodgkin disease and non-Hodgkin lymphomas. The thymus also contains another much less common type of cells called Kulchitsky cells, or neuroendocrine cells, which normally release certain hormones. These cells can give rise to cancers, called carcinoids or carcinoid tumors that often release the same type of hormones, and are similar to other tumors arising from neuroendocrine cells elsewhere in the body.

Common approaches to the treatment include surgery, immunotherapy, chemotherapy, radiation therapy, combination of chemotherapy and radiation therapy or biological therapy. Anticancer drugs that have been used in the treatment of thymomas and thymic carcinomas are doxorubicin (adriamycin), cisplatin, ifosfamide, and corticosteroids (prednisone). Often, these drugs are given in combination to increase their effectiveness. Combinations used to treat thymic cancer include cisplatin, doxorubicin, etoposide and cyclophosphamide, and the combination of cisplatin, doxorubicin, cyclophosphamide, and vincristine.

In some embodiments, described herein is a method of treating a thymus cancer comprising administering to a subject in need thereof a therapeutically effective amount of a compound of Formula (I), (Ia), (Ib), (Ic), (Id), (II), or (IIa).

In some embodiments, described herein is a method of treating hepatocellular carcinoma, prostate cancer, gastric adenocarcinoma, bladder cancer, papillary thyroid carcinoma, or non-small cell lung carcinoma in a subject in need thereof comprising administering to the subject in need thereof a therapeutically effective amount of a compound of Formula (I), (Ia), (Ib), (Ic), (Id), (II), or (IIa).

### Autoimmune Hepatitis

Hepatitis is an inflammatory process in the liver which can be caused by a variety of etiologies, including viruses and drugs. When a patient is suffering from a chronic hepatitis, but the cause of the disease is not known (i.e., following exclusion of other causes), and is associated with abnormalities in immunoregulation, the patient is said to have “autoimmune hepatitis”. Untreated, autoimmune hepatitis is progressive, and can result in liver failure and death.

Autoimmune hepatitis can further be classified as follows.

Type 1, or “classic” autoimmune hepatitis, is characterized in patients by the presence of antinuclear antibodies (ANA) in approximately 70% of such patients, the presence of anti-smooth muscle (anti-actin) antibodies (SMA) in more than 30% of such patients, and sensitivity to corticosteroids.

Type 2 autoimmune hepatitis is characterized by the presence of anti-liver-kidney-microsomal antibodies (ANTI-LKM-1), absence of ANA and SMA, and sensitivity to corticosteroids.

Type 3 autoimmune hepatitis patients are characterized by the presence by liver-pancreas antigen antibody (ANTI-LP) or anti-soluble liver antigen antibodies (ANTI-SLA), absence of ANA and ANTI-LKM-1, presence of SMA in 30% of such patients, and sensitivity to corticosteroids.

Type 4 autoimmune hepatitis patients are characterized as cryptogenic (tentative), and are characterized by the absence of ANA, SMA, ANTI-LKM-1, ANTI-SLA and ANTI-LP, and sensitivity to corticosteroids.

In some embodiments, described herein is a method of treating autoimmune hepatitis in a subject in need thereof comprising administering to the subject in need thereof a therapeutically effective amount of a compound of Formula (I), (Ia), (Ib), (Ic), (Id), (II), or (IIa).

### Non-Alcoholic Fatty Liver Disease (NAFLD)

NAFLD is a disorder affecting as many as 1 in 3-5 adults and 1 in 10 children in the United States, and refers to conditions where there is an accumulation of excess fat in the liver of people who drink little or no alcohol. The most common form of NAFLD is a non-serious condition called hepatic steatosis (fatty liver), in which fat accumulates in the liver cells: although this is not normal, by itself it probably does not damage the liver. NAFLD most often presents itself in individuals with a constellation of risk factors called the metabolic syndrome, which is characterized by elevated fasting plasma glucose (FPG) with or without intolerance to post-prandial glucose, being overweight or obese, high blood lipids such as cholesterol and triglycerides (TGs) and low high-density lipoprotein cholesterol (HDL-C) levels, and high blood pressure; but not all patients have all the manifestations of the metabolic syndrome. Obesity is thought to be the most common cause of NAFLD; and some experts estimate that about two-thirds of obese adults and one-half of obese children may have fatty liver. The majority of individuals with NAFLD have no symptoms and a normal physical examination (although the liver may be slightly enlarged); children may exhibit symptoms such as abdominal pain and fatigue, and may show patchy dark skin discoloration (acanthosis nigricans). The diagnosis of NAFLD is usually first suspected in an overweight or obese person who is found to have mild elevations in their liver blood tests during routine testing, though NAFLD can be present with normal liver blood tests, or incidentally detected on imaging investigations such as abdominal ultrasound or CT scan. It is confirmed by imaging studies, most commonly a liver ultrasound or magnetic resonance imaging (MRI), and exclusion of other causes.

In some embodiments, described herein is a method of treating non-alcoholic fatty liver disease (NAFLD) in a subject in need thereof comprising administering to the subject in need thereof a therapeutically effective amount of a compound of Formula (I), (Ia), (Ib), (Ic), (Id), (II), or (IIa).

### Kidney Injury and Lung Injury

Kidney injury takes many forms and can be life-threatening. Renal fibrosis is a direct consequence of the kidney's limited capacity to regenerate after injury. Renal scarring results in a progressive loss of renal function, ultimately leading to end-stage renal failure and a requirement for dialysis or kidney transplantation Mesangial cell hyperplasia is often a key feature of kidney or renal diseases and disorders. Such diseases and disorders may be caused by immunological or other mechanisms of injury, including IgAN, membranoproliferative glomerulonephritis or lupus nephritis. Imbalances in the control of mesangial cell replication also appear to play a key role in the pathogenesis of progressive renal failure. Renal fibrosis is the principal process underlying the progression of chronic kidney disease (CKD) to end-stage renal disease (ESRD).

Lung injury is a class of respiratory diseases in which scars are formed in the lung tissues, leading to serious breathing problems. Scar formation, the accumulation of excess fibrous connective tissue (the process called fibrosis), leads to thickening of the walls, and causes reduced oxygen supply in the blood. As a consequence patients suffer from perpetual shortness of breath. These diseases and disorders include but are not limited to idiopathic pulmonary fibrosis (IPF), secondary pulmonary hypertension (SPH), chronic thromboembolic pulmonary hypertension, lymphangioleiomyomatosis, and chronic obstructive pulmonary disease (COPD).

In some embodiments, described herein is a method of treating kidney injury or lung injury in a subject in need thereof comprising administering to the subject in need thereof a therapeutically effective amount of a compound of Formula (I), (Ia), (Ib), (Ic), (Id), (II), or (IIa). In some embodiments, the kidney injury is acute kidney injury.

### Myocardial Ischemia or Reperfusion Injury

Myocardial ischemic injury results from severe impairment of coronary blood supply and produces a spectrum of clinical syndromes. As a result of intensive investigation over decades, a detailed understanding is now available of the complexity of the response of the myocardium to an ischemic insult. Myocardial ischemia results in a characteristic pattern of metabolic and ultrastructural changes that lead to irreversible injury. Recent studies have explored the relationship of myocardial ischemic injury to the major modes of cell death, namely, oncosis and apoptosis. The evidence indicates that apoptotic and oncotic mechanisms can proceed together in ischemic myocytes with oncotic mechanisms and morphology dominating the end stage of irreversible injury. Myocardial infarcts evolve as a wavefront of necrosis, extending from subendocardium to subepicardium over a 3- to 4-hour period. A number of processes can profoundly influence the evolution of myocardial ischemic injury. Timely reperfusion produces major effects on ischemic myocardium, including a component of reperfusion injury and a greater amount of salvage of myocardium. Preconditioning by several short bouts of coronary occlusion and reperfusion can temporarily salvage significant amounts of myocardium and extend the window of myocardial viability.

In some embodiments, described herein is a method of treating myocardial ischemia or reperfusion injury in a subject in need thereof comprising administering to the subject in need thereof a therapeutically effective amount of a compound of Formula (I), (Ia), (Ib), (Ic), (Id), (II), or (IIa).

### Inflammation

Inflammation is a non-specific first reaction mounted by the immune system in response to a perceived injury or threat. It is an innate defensive response, distinguished from the more precisely tailored adaptive responses of the immune system. Inflammation may work cooperatively with adaptive responses of the immune system, which develop more slowly but are more precisely targeted to a harmful agent such as a chemical or pathogen that may be causing localized injury.

Inflammation may be associated with infections, but it occurs in response to virtually any type of injury or threat, including physical trauma, cold, burns from radiation, heat or corrosive materials, chemical irritants, bacterial or viral pathogens, localized oxygen deprivation (ischemia) or reperfusion (sudden reinfusion of oxygen to ischemic tissue), and others. It includes the classic symptoms of redness, heat, swelling, and pain, and may be accompanied by decreased function of the inflamed organ or tissue. It is a generalized reaction involving several effects that may tend to combat an injurious agent that may be present at the site where an injury or threat was detected, or it may tend to contain the injury or threat to its initial location, to keep it from spreading rapidly.

Adaptive immune responses, on the other hand, develop when the body is exposed to a particular harmful agent: the cellular immune system ‘learns’ to recognize and attack the particular harmful agent by developing cell-mediated responses. Then, if that harmful agent persists long enough or returns later, the adaptive system recognizes the harmful agent and attacks it with a very specific response directed at the harmful agent itself. Such adaptive responses take time to develop, but are usually extremely specific, while the innate responses like inflammation involve more general changes in the affected tissue, and are not specifically targeted at an agent that is causing injury. These innate reactions involve recruitment of protective cells and substances to the area of the injury, and, unlike the adaptive responses, they typically occur rapidly.

In some embodiments, described herein is a method of treating myocardial ischemia or reperfusion injury in a subject in need thereof comprising administering to the subject in need thereof a therapeutically effective amount of a compound of Formula (I), (Ia), (Ib), (Ic), (Id), (II), or (IIa).

### EXAMPLES

The following examples are intended to illustrate but not limit the disclosed embodiments.

### Example 1: Synthesis of N-((1R,3s,5S)-9-(2-((2-chlorophenyl)amino)-2-oxoethyl)-9-azabicyclo[3.3.1]nonan-3-yl)-3,4,5-trimethoxybenzamide

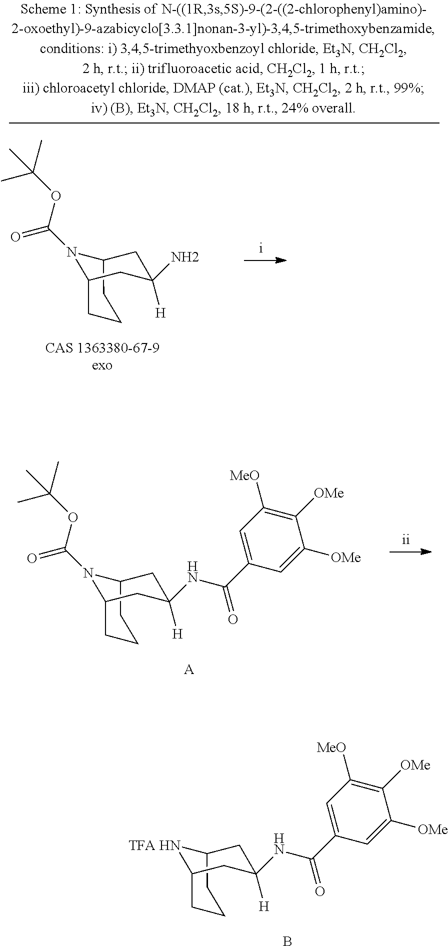

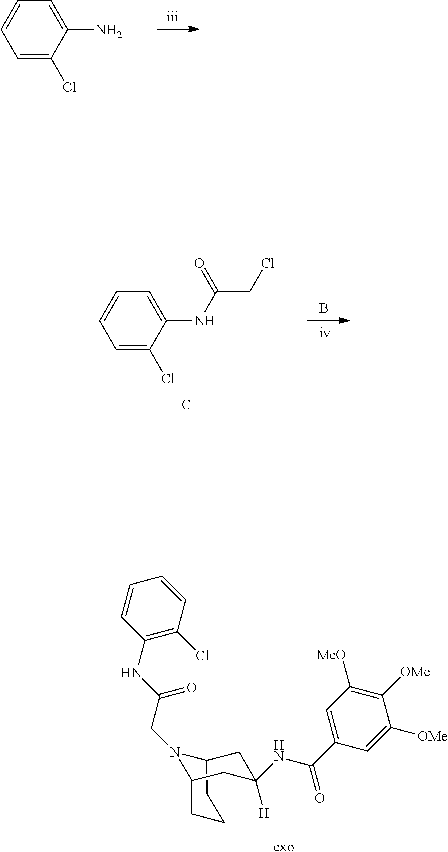

### Step i

exo-3-Amino-N-Boc-9-azabicyclo[3.3.1]nonane (CAS #1363380-67-9, 150 mg, 0.62 mmol) was dissolved in 5 mL of dichloromethane before charging with triethylamine (0.26 mL, 1.87 mmol) and 3,4,5-trimethoxybenzoyl chloride (173 mg, 0.75 mmol). After 2 h, the volatiles were evaporated in vacuo and the residue treated with 5 mL of water and extracted with 3×3 mL ethyl acetate. Concentration of the organics returned crude exo-(1R,3s,5S)-tert-butyl 3-(3,4,5-trimethoxybenzamido)-9-azabicyclo[3.3.1]nonane-9-carboxylate (A), which was used without purification. ¹H NMR (500 MHz, CDCl₃) δ 6.97 (s, 2H), 5.70 (d, J=8.1 Hz, 1H), 5.00 (dq, J=12.4, 6.1 Hz, 1H), 4.40 (br. s, 2H), 3.94 (s, 6H), 3.90 (s, 3H), 3.13 (qd, J=7.3, 4.8 Hz, 1H), 2.16 (dd, J=13.1, 5.9 Hz, 2H), 2.11-1.98 (m, 1H), 1.88 (tt, J=13.3, 5.9 Hz, 2H), 1.80-1.63 (m, 4H), 1.50 (s, 9H). MS (ESI+ve): Calculated for C₂₃H₃₅N₂O₆, [M+H]=435.24, observed [M+H]=435.29.

### Step ii

A, (0.62 mmol from the previous step) was dissolved in 4 mL of dichloromethane and treated with 4 mL of trifluoroacetic acid. After stirring for 1 h, the mixture was concentrated to afford exo-N-((1R,3s,5S)-9-azabicyclo[3.3.1]nonan-3-yl)-3,4,5-trimethoxybenzamide (B), as a colorless oil. The material was understood to be the trifluoroacetate salt, partly contaminated with residual trifluoroacetic acid, and was carried forward without purification. ¹H NMR (500 MHz, CDCl₃) δ 7.08 (s, 2H), 5.10 (dd, J=13.3, 6.4 Hz, 1H), 3.98-3.90 (m, 2H), 3.93 (s, 9H), 3.24 (qd, J=7.3, 4.8 Hz, 1H), 2.50-2.27 (m, 4H), 2.27-2.03 (m, 4H), 2.03-1.85 (m, 1H). MS (ESI+ve): Calculated for C₁₈H₂₇N₂O₄, [M+H]=335.41, observed [M+H]=335.22.

### Step iii

A solution of 2-chloroacetyl chloride (0.33 mL, 4.15 mmol) in 4 mL dichloromethane was treated with a solution of 2-chloroaniline (0.43 mL, 4.1 mmol), N,N-dimethylpyridin-4-amine (5 mg, cat.) and triethylamine (0.58 mL, 4.16 mmol) in 4 mL dichloromethane dropwise under a nitrogen atmosphere. The mixture was stirred for 2 h and the solvent was removed in vacuo. The residue was taken up in 50 mL ethyl acetate and was washed with aqueous sodium bisulfate. The organic phase was dried over sodium sulfate and concentrated in vacuo to provide 2-chloro-N-(2-chlorophenyl)acetamide C (832 mg, 99%) as a tan solid which was used without further purification. ¹H NMR (500 MHz, CDCl₃) δ 8.96 (s, 1H), 8.39 (dd, J=8.3, 1.2 Hz, 1H), 7.44 (dd, J=8.0, 1.4 Hz, 1H), 7.36-7.31 (m, 1H), 7.13 (td, J=7.9, 1.5 Hz, 1H), 4.27 (s, 2H).

### Step iv

Crude B from Step ii (max 0.62 mmol) was dissolved in 1.5 mL of dichloromethane before charging with triethylamine (0.88 mL, 6.29 mmol) and solid C from Step iii (192 mg, 0.94 mmol). After stirring for 18 h, the mixture was concentrated and the residue was purified via silica gel flash chromatography, eluting with 0-60% ethyl acetate/hexane, to afford 74 mg (24% overall yield for the 3 step sequence) of N-((1R,3s,5S)-9-(2-((2-chlorophenyl)amino)-2-oxoethyl)-9-azabicyclo[3.3.1]nonan-3-yl)-3,4,5-trimethoxybenzamide. ¹H NMR (500 MHz, DMSO-d6) δ 10.26 (s, 1H), 8.41 (dd, J=8.2, 1.6 Hz, 1H), 8.04 (d, J=7.9 Hz, 1H), 7.53 (dd, J=8.0, 1.4 Hz, 1H), 7.36 (td, J=8.4, 7.9, 1.5 Hz, 1H), 7.17-7.09 (m, 3H), 4.80-4.66 (m, 1H), 3.84 (s, 6H), 3.70 (s, 3H), 3.48 (s, 2H), 3.00 (s, 2H), 2.11-1.99 (m, 2H), 1.99-1.88 (m, 2H), 1.88-1.80 (m, 1H), 1.78-1.66 (m, 5H); ¹³C NMR (125 MHz, DMSO-d6) δ 169.51, 164.90, 152.50, 139.92, 134.50, 130.00, 129.26, 127.95, 124.60, 121.89, 120.24, 104.88, 60.06, 57.28, 56.06, 52.39, 42.65, 31.82, 29.18, 19.64; HRMS (ESI+ve): Calculated for C₂₆H₃₂ClN₃O₅, [M+H]=502.2103, observed [M+H]=502.2099.

### Example 2: Synthesis of N-((1R,3s,5S)-9-((4-chlorobenzo[d]thiazol-2-yl)methyl)-9-azabicyclo[3.3.1]nonan-3-yl)-3,4,5-trimethoxybenzamide

The title compound was synthesized using the procedure described in Example 1. Crude N-((1R,3s,5S)-9-azabicyclo[3.3.1]nonan-3-yl)-3,4,5-trimethoxybenzamide (max. 20.0 mg, 46.4 mmol) was dissolved in MeCN (2 mL) and then sequentially treated with 4-chloro-2-(chloromethyl)benzo[d]thiazole (10.6 mg, 48.7 mmol) and K₂CO₃ (19 mg, 139 mmol). The mixture was then stirred at 70° C. for 3 h. The solution was diluted with water and then extracted with CH₂Cl₂. Concentration of the organic portion was followed by purification on silica gel (hexane/EtOAc gradient 10%-100%) gave N-((1R,3s,5S)-9-((4-chlorobenzo[d]thiazol-2-yl)methyl)-9-azabicyclo[3.3.1]nonan-3-yl)-3,4,5-trimethoxybenzamide (10.7 mg, 45%). ¹H NMR (500 MHz, Chloroform-d) δ 7.77 (dd, J=8.0, 1.1 Hz, 1H), 7.49 (dd, J=7.8, 1.1 Hz, 1H), 7.31 (d, J=7.9 Hz, 1H), 7.03 (s, 2H), 5.87 (d, J=8.0 Hz, 1H), 5.02-4.86 (m, 1H), 4.29 (s, 2H), 3.95 (s, 6H), 3.91 (s, 3H), 3.21-3.07 (m, 2H), 2.18-1.98 (m, 5H), 1.95-1.84 (m, 2H), 1.84-1.73 (m, 1H), 1.70-1.60 (m, 3H). LRMS (ESI+ve): Calculated for C₂₆H₃₁ClN₃O₄S, [M+H]=516.17, observed [M+H]=516.46.

### Example 3: Synthesis of 3,4,5-trimethoxy-N-((1R,3s,5S)-9-((4-(trifluoromethyl)benzo[d]thiazol-2-yl)methyl)-9-azabicyclo[3.3.1]nonan-3-yl)benzamide (Compound 1)

The title compound was synthesized using the procedure described in Example 1. Crude N-((1R,3s,5S)-9-azabicyclo[3.3.1]nonan-3-yl)-3,4,5-trimethoxybenzamide (max. 25.0 mg, 58.0 mmol) was dissolved in MeCN (2 mL) and then sequentially treated with 2-(chloromethyl)-4-(trifluoromethyl)benzo[d]thiazole (15.3 mg, 60.8 mmol) and K₂CO₃ (24.0 mg, 173.8 mmol). The mixture was then stirred at 70° C. for 3 h. The solution was diluted with water and then extracted with CH₂Cl₂. Concentration of the organic portion was followed by purification on silica gel (hexane/EtOAc gradient 10%-100%) gave 3,4,5-trimethoxy-N-((1R,3s,5S)-9-((4-(trifluoromethyl)benzo[d]thiazol-2-yl)methyl)-9-azabicyclo[3.3.1]nonan-3-yl)benzamide (Compound 1) (18.6 mg, 58%); ¹H NMR (500 MHz, Chloroform-d): δ 8.06 (d, J=8.0 Hz, 1H), 7.76 (d, J=7.5 Hz, 1H), 7.44 (t, J=7.8 Hz, 1H), 7.03 (s, 2H), 5.95-5.80 (m, 1H), 5.03-4.86 (m, 1H), 4.30 (s, 2H), 3.95 (s, 6H), 3.91 (s, 3H), 3.22-3.07 (m, 2H), 2.21-1.98 (m, 5H), 1.94-1.84 (m, 2H), 1.83-1.75 (m, 1H), 1.73-1.63 (m, 2H). ¹³C NMR (126 MHz, CDCl₃): δ 166.46, 153.14, 150.02, 140.82, 137.13, 130.06, 126.98, 125.59, 124.81, 123.73, 123.58, 123.33, 123.26, 123.23, 123.19, 123.15, 123.07, 122.64, 120.47, 104.33, 60.87, 56.29, 55.41, 52.96, 43.42, 34.11, 27.43, 19.80. LRMS (ESI+ve): Calculated for C₂₇H₃₁F₃N₃O₄S, [M+H]=550.20, observed [M+H]=550.69.

### Example 4: Synthesis of N-((1R,3s,5S)-9-(benzo[b]thiophen-2-ylmethyl)-9-azabicyclo[3.3.1]nonan-3-yl)-3,4,5-trimethoxybenzamide

The title compound was synthesized using the procedure described in Example 1. Crude N-((1R,3s,5S)-9-azabicyclo[3.3.1]nonan-3-yl)-3,4,5-trimethoxybenzamide (max. 65.0 mg, 0.150 mmol) was dissolved in MeCN (2 mL) and then sequentially treated with 2-(chloromethyl)benzo[b]thiophene (0.15 mmol) and K₂CO₃ (62 mg, 0.45 mmol). The mixture was then stirred at 70° C. for 3 h. The solution was diluted with water and then extracted with CH₂Cl₂. Concentration of the organic portion was followed by purification on silica gel (hexane/EtOAc gradient 10%-100%) gave N-((1R,3s,5S)-9-(benzo[b]thiophen-2-ylmethyl)-9-azabicyclo[3.3.1]nonan-3-yl)-3,4,5-trimethoxybenzamide (47 mg) as a white solid. ¹H NMR (500 MHz, DMSO-d6) δ 8.11 (d, J=8.0 Hz, 1H), 7.88 (d, J=7.8 Hz, 1H), 7.74 (d, J=7.9 Hz, 1H), 7.36-7.26 (m, 3H), 7.19 (s, 2H), 4.76 (m, 1H), 4.17 (s, 2H), 3.85 (s, 6H), 3.71 (s, 3H), 3.01 (s, 2H), 1.97 (m, 4H), 1.93-1.82 (m, 1H), 1.73 (m, 3H), 1.59-1.43 (m, 2H). LCMS (ESI+) for C₂₇H₃₂N₂O₄S [M+H] expected=481.21, found=481.40.

### Example 5: Synthesis of 4-isopropoxy-3-methoxy-N-((1R,3s,5S)-9-((4-(trifluoromethyl)benzo[d]thiazol-2-yl)methyl)-9-azabicyclo[3.3.1]nonan-3-yl)benzamide

The title compound was synthesized using the procedure described in Example 1. exo-tert-Butyl (9-azabicyclo[3.3.1]nonan-3-yl) carbamate (CAS #1363380-67-9; 50.0 mg, 0.208 mmol) was dissolved in MeCN (2 mL) and then sequentially treated with 2-(chloromethyl)-4-(trifluoromethyl)benzo[d]thiazole (52.4, 0.208 mmol) and K₂CO₃ (86 mg, 0.624 mmol). The mixture was then stirred at 70° C. for 3 h. The solution was diluted with water and then extracted with CH₂Cl₂. Concentration of the organic portion gave crude exo-tert-butyl (9-((4-(trifluoromethyl)benzo[d]thiazol-2-yl)methyl)-9-azabicyclo[3.3.1]nonan-3-yl)carbamate (max 94.8 mg, 0.208 mmol) was dissolved in dichloromethane (1 mL). Trifluoroacetic acid (1 mL) was then added and the resulting solution was stirred for 2 h. Concentration in vacuo then gave a crude exo-9-((4-(trifluoromethyl)benzo[d]thiazol-2-yl)methyl)-9-azabicyclo[3.3.1]nonan-3-aminium 2,2,2-trifluoroacetate (max 0.208 mmol). 4-isopropoxy-3-methoxybenzoic acid (CAS #3535-33-9; 8.36 mg, 39.8 mmol) was dissolved in MeCN (1 mL) and sequentially treated with HATU (18.9 mg, 49.7 mmol), triethyl amine (15 mL, 99.5 mmol) and crude exo-9-((4-(trifluoromethyl)benzo[d]thiazol-2-yl)methyl)-9-azabicyclo[3.3.1]nonan-3-aminium 2,2,2-trifluoroacetate (max 15 mg, 33.2 mmol). The mixture was stirred overnight at room temperature. The solution was diluted with water and then extracted with CH₂Cl₂. Concentration of the organic portion was followed by purification on silica gel (hexane/EtOAc gradient 10%-100%) gave 4-isopropoxy-3-methoxy-N-((1R,3s,5S)-9-((4-(trifluoromethyl)benzo[d]thiazol-2-yl)methyl)-9-azabicyclo[3.3.1]nonan-3-yl)benzamide (6.8 mg, 37%) as a white solid; ¹H NMR (500 MHz, Chloroform-d) δ 8.07 (d, J=8.0 Hz, 1H), 7.76 (d, J=7.5 Hz, 1H), 7.46-7.41 (m, 2H), 7.27 (d, J=2.1 Hz, 1H), 6.92 (d, J=8.4 Hz, 1H), 5.85 (d, J=7.9 Hz, 1H), 5.04-4.87 (m, 1H), 4.64 (hept, J=6.1 Hz, 1H), 4.31 (s, 2H), 3.94 (s, 3H), 3.14 (s, 2H), 2.19-1.97 (m, 5H), 1.93-1.85 (m, 2H), 1.83-1.76 (m, 1H), 1.73-1.64 (m, 2H), 1.42 (d, J=6.1 Hz, 6H); LRMS (ESI+ve): Calculated for C₂₈H₃₂F₃N₃O₃S, [M+H]=548.22, observed [M+H]=548.56.

### Example 6: Synthesis of 4-isopropoxy-3-methoxy-N-((1R,3s,5S)-9-((4-(trifluoromethyl)benzo[d]thiazol-2-yl)methyl)-9-azabicyclo[3.3.1]nonan-3-yl)benzamide

The title compound was synthesized using the procedure described in Example 5. 4-(sec-Butoxy)-3-methoxybenzoic acid (CAS #52009-56-0; 8.6 mg, 38.3 mmol) was dissolved in MeCN (1 mL) and sequentially treated with HATU (18.2 mg, 47.9 mmol), triethyl amine (13.4 mL, 96 mmol) and crude exo-9-((4-(trifluoromethyl)benzo[d]thiazol-2-yl)methyl)-9-azabicyclo[3.3.1]nonan-3-aminium 2,2,2-trifluoroacetate (max 15 mg, 33.2 mmol). The mixture was stirred overnight at room temperature. The solution was diluted with water and then extracted with CH₂Cl₂. Concentration of the organic portion was followed by purification on silica gel (hexane/EtOAc gradient 10%-100%) gave 4-(sec-butoxy)-3-methoxy-N-((1R,3s,5S)-9-((4-(trifluoromethyl)benzo[d]thiazol-2-yl)methyl)-9-azabicyclo[3.3.1]nonan-3-yl)benzamide (5.8 mg, 32%) as a white solid; ¹H NMR (500 MHz, Chloroform-d) δ 8.07 (d, J=8.0 Hz, 1H), 7.75 (d, J=7.5 Hz, 1H), 7.47-7.41 (m, 2H), 7.27 (d, J=2.0 Hz, 1H), 6.91 (d, J=8.4 Hz, 1H), 5.86 (d, J=8.0 Hz, 1H), 5.03-4.87 (m, 1H), 4.38 (p, J=6.2 Hz, 1H), 4.31 (s, 2H), 3.94 (s, 3H), 3.15 (d, J=4.9 Hz, 2H), 2.19-2.00 (m, 5H), 1.93-1.83 (m, 3H), 1.83-1.75 (m, 1H), 1.73-1.63 (m, 4H), 1.37 (d, J=6.0 Hz, 3H), 1.02 (t, J=7.4 Hz, 3H); LRMS (ESI+ve): Calculated for C₂₉H₃₄F₃N₃O₃S, [M+H]=562.24, observed [M+H]=562.39.

### Example 7: Synthesis of N-((1R,3s,5S)-9-((4,5-dichlorobenzo[d]thiazol-2-yl)methyl)-9-azabicyclo[3.3.1]nonan-3-yl)-3,4,5-trimethoxybenzamide

The title compound was synthesized using the procedure described in Example 1. Crude N-((1R,3s,5S)-9-azabicyclo[3.3.1]nonan-3-yl)-3,4,5-trimethoxybenzamide (max. 15.0 mg, 33.5 mmol) was dissolved in MeCN (2 mL) and then sequentially treated with 4,5-dichloro-2-(chloromethyl)benzo[d]thiazole (8.5 mg, 33.5 mmol) and K₂CO₃ (23 mg, 168 mmol). The mixture was then stirred at 70° C. for 3 h. The solution was diluted with water and then extracted with CH₂Cl₂. Concentration of the organic portion was followed by purification on silica gel (hexane/EtOAc gradient 10%-100%) gave N-((1R,3s,5S)-9-((4,5-dichlorobenzo[d]thiazol-2-yl)methyl)-9-azabicyclo[3.3.1]nonan-3-yl)-3,4,5-trimethoxybenzamide (6.0 mg, 33%) as white solid; ¹H NMR (500 MHz, Chloroform-d) δ 7.69 (dd, J=8.5, 1.7 Hz, 1H), 7.45 (dd, J=8.5, 1.7 Hz, 1H), 7.03 (d, J=1.7 Hz, 2H), 5.89 (d, J=8.0 Hz, 1H), 5.03-4.84 (m, 1H), 4.27 (s, 2H), 3.95 (s, 6H), 3.91 (s, 3H), 3.12 (s, 2H), 2.17-1.96 (m, 5H), 1.93-1.82 (m, 2H), 1.82-1.74 (m, 1H), 1.71-1.63 (m, 3H); LRMS (ESI+ve): Calculated for C₂₆H₂₉C₁₂N₃O₄S, [M+H]=550.13, observed [M+H]=550.62.

### Example 8: ((1R,5S)-9-((4-(trifluoromethyl)benzo[d]thiazol-2-yl)methyl)-3,9-diazabicyclo[3.3.1]nonan-3-yl)(3,4,5-trimethoxyphenyl)methanone

The title compound was synthesized using the procedure described in Example 1. tert-Butyl 3,9-diazabicyclo[3.3.1]nonane-9-carboxylate (25 mg, 110.5 mmol) was charged into a vial, dissolved in MeCN (2 mL) and sequentially treated with 3,4,5-trimethoxybenzoyl chloride (25.5 mg, 110.5 mmol) and triethyl amine (46 mL, 331.5 mmol) and the resulting mixture stirred at room temperature overnight. The mixture was diluted with ethyl acetate (10 mL) and washed with sat.sodium bicarbonate solution and brine. The organic layer was dried and the crude product was dissolved in dichloromethane (1 mL). Trifluoroacetic acid (1 mL) was then added and the resulting solution was stirred for 2 h. Concentration in vacuo then gave a residue (max. 25.0 mg, 55.8 mmol) was dissolved in MeCN (2 mL) and then sequentially treated with 2-(chloromethyl)-4-(trifluoromethyl)benzo[d]thiazole (14.6 mg, 58 mmol) and K₂CO₃ (23.0 mg, 165.7 mmol). The mixture was then stirred at 70° C. for 3 h. The solution was diluted with water and then extracted with CH₂Cl₂. Concentration of the organic portion was followed by purification on silica gel (hexane/EtOAc gradient 10%-100%) gave ((1R,5S)-9-((4-(trifluoromethyl)benzo[d]thiazol-2-yl)methyl)-3,9-diazabicyclo[3.3.1]nonan-3-yl)(3,4,5-trimethoxyphenyl)methanone (13.8 mg, 47%) as a white solid; ¹H NMR (400 MHz, Chloroform-d) δ 8.08 (d, J=8.0 Hz, 1H), 7.76 (d, J=7.6 Hz, 1H), 7.45 (t, J=7.8 Hz, 1H), 6.64 (s, 2H), 4.78-4.59 (m, 1H), 4.39 (s, 2H), 3.90 (s, 6H), 3.88 (s, 3H), 3.82-3.65 (m, 2H), 3.47-3.38 (m, 1H), 3.17-3.06 (m, 1H), 2.96-2.85 (m, 1H), 2.23-2.08 (m, 3H), 1.88-1.69 (m, 2H), 1.60-1.51 (m, 1H); LRMS (ESI+ve): Calculated for C₂₆H₂₈F₃N₃O₄S, [M+H]=536.18, observed [M+H]=536.37.

### PHARMACEUTICAL COMPOSITIONS

### Example A-1: Parenteral Composition

To prepare a parenteral pharmaceutical composition suitable for administration by injection, 100 mg of a water-soluble salt of a compound of Formula (I), (Ia), (Ib), (Ic), (Id), (II), or (IIa), or pharmaceutically acceptable solvate or N-oxide thereof, is dissolved in 2% HPMC, 1% Tween 80 in DI water, pH 2.2 with MSA, q.s. to at least 20 mg/mL. The mixture is incorporated into a dosage unit form suitable for administration by injection.

### Example A-2: Oral Composition

To prepare a pharmaceutical composition for oral delivery, 100 mg of a compound of Formula (I), (Ia), (Ib), (Ic), (Id), (II), or (IIa), pharmaceutically acceptable solvate or N-oxide thereof, is mixed with 750 mg of starch. The mixture is incorporated into an oral dosage unit for, such as a hard gelatin capsule, which is suitable for oral administration.

### BIOLOGY EXAMPLES

### Example B-1: In Vitro Studies

### Primary Assays

Compounds of Formula (I), (Ia), (Ib), (Ic), (Id), and (II) are tested for their ability to inhibit β-arrestin recruitment in CXCR6-expressing CHO-K1 cells using the β-galactosidase PathHunter Enzyme Complementation Platform from DiscoveRx. A detailed protocol for this assay is available on PubChem (AIDs 624129). Accordingly, 800 CHO-K1 cells expressing CXCR6 (DiscoveRx, Cat #93-0205C₂) are seeded into the wells of a white opaque 1536-well plate, centrifuged, and incubated overnight at 37° C., 5% CO₂. The next day, compounds of Formula (I), (Ia), (Ib), (Ic), (Id), and (II) and controls (CXCL16 (10 nM) or DMSO) are dispensed into wells and incubated for 1.5 h. The assay is terminated upon addition of lysis buffer, detection substrate, and enzyme. Luminescence is read on the Envision plate reader 1 h post-termination. All compounds are tested in a 10 pt dose-response curve in duplicate. Compounds will also be tested in a CXCR6-dependent cAMP assay using the Lance Ultra assay from PerkinElmer. Accordingly, 2 uL CXCR6-expressing cells are suspended in assay media (OptiMEM; 2% FBS; 1×P/S) and plated at 500 cells/well in 1536-well, white, TC plates and grown overnight at 37° C.; 5% CO₂. 40 nL test compounds of Formula (I), (Ia), (Ib), (Ic), (Id), and (II) are added to cells using ECHO dispenser. 2 uL of 2.5 mM forskolin in stimulation buffer (HBSS; 5 mM HEPES; 0.1% BSA; 0.1 mM RO-20-1724) with or without 20 nM CXCL16 is transferred to wells using BioRAPTR. Wells treated with forskolin and CXCL16 serve as negative controls and wells treated with forskolin only serve as positive controls. Cells are incubated at room temperature for 30 min followed by addition of 2 uL each EU-cAMP tracer and ULight anti-cAMP Ab in detection buffer according to manufacturer's instruction. After 30 min at room temperature, TR-FRET is read using EnVision plate reader.

### Counter Screen to Determine Compound Selectivity

Compounds of Formula (I), (Ia), (Ib), (Ic), (Id), and (II) are tested against a panel of chemokine receptors using the DiscoveRx β-arrestin cell-based format. Assays for the CCR6, CXCR4 and CXCR5 receptors using the same assay conditions as detailed in protocols are available on PubChem (AIDs 651941, 651902, 651594, respectively). In addition, compounds will be tested against murine CXCR6 to determine species selectivity.

### Secondary Confirmatory Assay

Validated compounds of Formula (I), (Ia), (Ib), (Ic), (Id), and (II) will be further confirmed for their ability to block an agonist-induced functional response of the cells expressing CXCR6 as monitored by label-free cell impedance readout. 100 uL CXCR6 β-arrestin cells suspended in assay media (OptiMEM; 2% FBS; 1×P/S) are plated in 96-well xCELLigence E-Plates (ACEA) at a cell density of 20,000 cells/well and grown overnight at 37° C.; 5% CO₂. CXCL16 with or without test compounds are added to cells and average cell index is continuously monitored every minute for 30 minutes. Negative controls receive media with DMSO at a final concentration of 0.1%. Changes in impedance cell index values are used to quantify the effect of CXCL16 and test compounds on cell physiology.

### Secondary Assay of In Vitro Efficacy

In addition to the CXCR6 assays described, compound efficacy by in vitro assay of chemotaxis will be assessed. Detecting chemotaxis of the CXCR6 receptor has proven challenging in endogenous expressing cell lines. A robust 96-well cell-based assay in CCRF-CEM acute lymphoblastic leukemia cell line for chemotaxis toward CXCL12, the endogenous ligand for the CXCR4 receptor using a Corning Transwell insert has been established. However, migration with the CXCL16 ligand was not detected even upon induction with IL2. In PC3 cells, a two-fold signal for CXCL16 migration was detected. Transfection of the CXCR6 cDNA into Jurkat cells and employ our 96-well Transwell format to measure chemotaxis toward CXCL16 and subsequent block by compounds will be performed. The current chemotaxis assay in lymphoblasts will serve as an important functional selectivity assay to indicate off-target activity for CXCR4.

Representative data for exemplary compounds disclosed in Table 1 is presented in the following table B-1.

TABLE B-1

| | IC₅₀ | IC₅₀ |
|---|---|---|
| Compound | (β-arrestin) | (cAMP) |
| N-((1R,3s,5S)-9-(2-((2-chlorophenyl)amino)-2-oxoethyl)-9- | B | C |
| azabicyclo[3.3.1]nonan-3-yl)-3,4,5-trimethoxybenzamide | | |
| N-((1R,3s,5S)-9-((4-chlorobenzo[d]thiazol-2-yl)methyl)-9- | B | B |
| azabicyclo[3.3.1]nonan-3-yl)-3,4,5-trimethoxybenzamide | | |
| 3,4,5-trimethoxy-N-((1R,3s,5S)-9-((4- | A | B |
| (trifluoromethyl)benzo[d]thiazol-2-yl)methyl)-9- | | |
| azabicyclo[3.3.1]nonan-3-yl)benzamide (Compound 1) | | |
| N-((1R,3s,5S)-9-(benzo[b]thiophen-2-ylmethyl)-9- | B | D |
| azabicyclo[3.3.1]nonan-3-yl)-3,4,5-trimethoxybenzamide | | |
| 4-isopropoxy-3-methoxy-N-((1R,3s,5S)-9-((4- | B | B |
| (trifluoromethyl)benzo[d]thiazol-2-yl)methyl)-9- | | |
| azabicyclo[3.3.1]nonan-3-yl)benzamide | | |
| 4-(sec-butoxy)-3-methoxy-N-((1R,3s,5S)-9-((4- | A | ND |
| (trifluoromethyl)benzo[d]thiazol-2-yl)methyl)-9- | | |
| azabicyclo[3.3.1]nonan-3-yl)benzamide | | |
| N-((1R,3s,5S)-9-((4,5-dichlorobenzo[d]thiazol-2-yl)methyl)-9- | A | ND |
| azabicyclo[3.3.1]nonan-3-yl)-3,4,5-trimethoxybenzamide | | |

Where A = <100 nM;
B = 100 nM-2 μM;
C = 2-5 μM;
D = 5-20 μM;
E = >20 μM;
ND = no data

Representative data for exemplary compound disclosed in Table 2 is presented in the following table B-2.

TABLE B-2

| | IC₅₀ | IC₅₀ |
|---|---|---|
| Compound | (β-arrestin) | (cAMP) |
| ((1R,5S)-9-((4-(trifluoromethyl)benzo[d]thiazol- | B | D |
| 2-yl)methyl)-3,9-diazabicyclo[3.3.1]nonan-3- | | |
| yl)(3,4,5-trimethoxyphenyl)methanone | | |

Where A = <100 nM;
B = 100 nM-2 μM;
C = 2-5 μM;
D = 5-20 μM;
E = >20 μM;
ND = no data

The examples and embodiments described herein are for illustrative purposes only and various modifications or changes suggested to persons skilled in the art are to be included within the spirit and purview of this application and scope of the appended claims.

1. A compound of Formula (I), or a pharmaceutically acceptable salt, solvate, or N-oxide thereof: 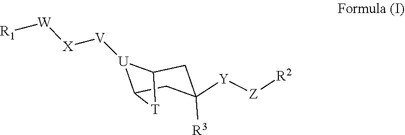
wherein,
U is N;
V is —CH₂—;
T is —(CH)ₙ—;
Y is —C(═O)NR⁴—, —NR⁴C(═O)—, —OC(═O)NR⁴—, —NR⁴C(═O)O—, —NR⁴C(═O)NR⁴—, —S(═O)₂NR⁴—, or —NR⁴S(═O)₂—;
W, X, and Z are absent;
R¹ is substituted or unsubstituted heteroaryl;
R² is an unsubstituted phenyl or a substituted phenyl that is substituted with 1, 2, 3, 4, or 5 R⁷;
R³ is H, D, F, substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, substituted or unsubstituted C₂-C₉ heterocycloalkyl, substituted or unsubstituted aryl, or substituted or unsubstituted heteroaryl;
each R⁴ is independently H, D, or substituted or unsubstituted C₁-C₆ alkyl;
n is 1, 2, 3, or 4; and
each R⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, or substituted or unsubstituted C₂-C₉ heterocycloalkyl.

2-4. (canceled)

5. The compound of claim 1, or a pharmaceutically acceptable salt, solvate, or N-oxide thereof, wherein R¹ is substituted or unsubstituted bicyclic heteroaryl.

6. The compound of claim 5, or a pharmaceutically acceptable salt, solvate, or N-oxide thereof, wherein R¹ is selected from the group consisting of: 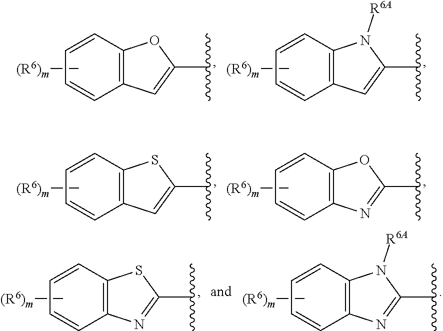
wherein each R⁶ is independently D, halogen, substituted or unsubstituted C₁-C₄ alkyl, substituted or unsubstituted C₁-C₄ fluoroalkyl, or substituted or unsubstituted C₁-C₄ heteroalkyl;
R^{6A} is H, D, substituted or unsubstituted C₁-C₄ alkyl, or substituted or unsubstituted C₁-C₄ fluoroalkyl; and
m is 0, 1, 2, 3, 4, or 5.

7. (canceled)

8. The compound of claim 1, or a pharmaceutically acceptable salt, solvate, or N-oxide thereof, wherein n is 2, 3, or 4.

9. The compound of claim 1, or a pharmaceutically acceptable salt, solvate, or N-oxide thereof, wherein R³ is H.

10. The compound of claim 1, or a pharmaceutically acceptable salt, solvate, or N-oxide thereof, wherein Y is —C(═O)NR⁴—, —NR⁴C(═O)—, or —NR⁴S(═O)₂—.

11. (canceled)

12. (canceled)

13. The compound of claim 1, or a pharmaceutically acceptable salt, solvate, or N-oxide thereof, wherein R² is 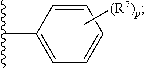
wherein each R⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₁₀ cycloalkyl, or substituted or unsubstituted C₂-C₉ heterocycloalkyl; and
p is 0, 1, 2, 3, 4, or 5.

14. The compound of claim 1, or a pharmaceutically acceptable salt, solvate, or N-oxide thereof, wherein each R⁷ is independently selected from the group consisting of -OMe, -OEt, -On-Pr, -Oi-Pr, -On-butyl, -Osec-butyl, and -Oi-butyl.

15. The compound of claim 14, or a pharmaceutically acceptable salt, solvate, or N-oxide thereof, wherein p is 2 or 3.

16-18. (canceled)

19. The compound of claim 1, or a pharmaceutically acceptable salt, solvate, or N-oxide thereof, wherein:
R¹— is

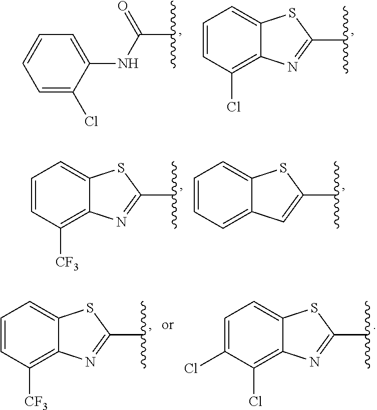

20. The compound of claim 1, or a pharmaceutically acceptable salt, solvate, or N-oxide thereof, wherein:
R² is

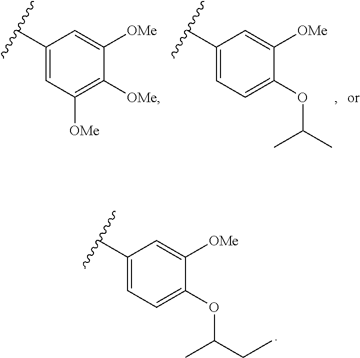 [SMILES: *c1cc(OC)c(OC)c(OC)c1 ; *c1ccc(OC(C)C)c(OC)c1 ; *c1ccc(OC(C)CC)c(OC)c1]

21. The compound of claim 1, that is:
N-((1R,3s,5S)-9-((4-chlorobenzo[d]thiazol-2-yl)methyl)-9-azabicyclo[3.3.1]nonan-3-yl)-3,4,5-trimethoxybenzamide;
3,4,5-trimethoxy-N-((1R,3s,5S)-9-((4-(trifluoromethyl)benzo[d]thiazol-2-yl)methyl)-9-azabicyclo[3.3.1]nonan-3-yl)benzamide;
N-((1R,3s,5S)-9-(benzo[b]thiophen-2-ylmethyl)-9-azabicyclo[3.3.1]nonan-3-yl)-3,4,5-trimethoxybenzamide;
4-isopropoxy-3-methoxy-N-((1R,3s,5S)-9-((4-(trifluoromethyl)benzo[d]thiazol-2-yl)methyl)-9-azabicyclo[3.3.1]nonan-3-yl)benzamide;
4-(sec-butoxy)-3-methoxy-N-((1R,3s,5S)-9-((4-(trifluoromethyl)benzo[d]thiazol-2-yl)methyl)-9-azabicyclo[3.3.1]nonan-3-yl)benzamide; or
N-((1R,3s,5S)-9-((4,5-dichlorobenzo[d]thiazol-2-yl)methyl)-9-azabicyclo[3.3.1]nonan-3-yl)-3,4,5-trimethoxybenzamide;
or a pharmaceutically acceptable salt, solvate, or N-oxide thereof.

22. A compound of Formula (II), or a pharmaceutically acceptable salt, solvate, or N-oxide thereof: 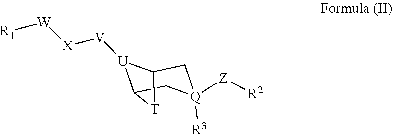
wherein,
U and Q are N;
V is —CH₂—;
T is —(CH₂)ₙ—;
W, X, and Z are absent;
R¹ is substituted or unsubstituted heteroaryl;
R² is an unsubstituted phenyl or a substituted phenyl that is substituted with 1, 2, 3, 4, or 5 R⁷;
each R⁴ is independently H, D, or substituted or unsubstituted C₁-C₆ alkyl; and
each R⁵ is independently H, D, F, substituted or unsubstituted C₁-C₆ alkyl, substituted or unsubstituted C₁-C₆ fluoroalkyl, substituted or unsubstituted C₁-C₆ heteroalkyl, substituted or unsubstituted aryl, or substituted or unsubstituted heteroaryl; and
n is 1, 2, 3, or 4.

23-41. (canceled)

42. The compound of claim 22, that is:
((1R,5S)-9-((4-(trifluoromethyl)benzo[d]thiazol-2-yl)methyl)-3,9-diazabicyclo[3.3.1]nonan-3-yl)(3,4,5-trimethoxyphenyl)methanone;
or a pharmaceutically acceptable salt, solvate, or N-oxide thereof.

43. A pharmaceutical composition comprising a compound of claim 1, or a pharmaceutically acceptable salt, solvate, or N-oxide thereof, and at least one pharmaceutically acceptable excipient.

44. A pharmaceutical composition comprising a compound of claim 22, or a pharmaceutically acceptable salt, solvate, or N-oxide thereof, and at least one pharmaceutically acceptable excipient.

45-48. (canceled)

49. A method of treating cancer in a subject in need thereof comprising administering to the subject in need thereof a therapeutically effective amount of a compound of claim 1, or a pharmaceutically acceptable salt, solvate, or N-oxide thereof, wherein the cancer is selected from the group consisting of hepatocellular carcinoma, prostate cancer, gastric adenocarcinoma, bladder cancer, papillary thyroid carcinoma, and non-small cell lung carcinoma.

50. (canceled)

51. A method of treating autoimmune hepatitis in a subject in need thereof comprising administering to the subject in need thereof a therapeutically effective amount of a compound of claim 1, or a pharmaceutically acceptable salt, solvate, or N-oxide thereof.

52. A method of treating kidney injury or lung injury in a subject in need thereof comprising administering to the subject in need thereof a therapeutically effective amount of a compound of claim 1, or a pharmaceutically acceptable salt, solvate, or N-oxide thereof, wherein the kidney injury is acute kidney injury.

53. (canceled)

54. A method of treating inflammation, myocardial ischemia or reperfusion injury in a subject in need thereof comprising administering to the subject in need thereof a therapeutically effective amount of a compound of claim 1, or a pharmaceutically acceptable salt, solvate, or N-oxide thereof.

55. (canceled)
